(19) United States
(12) Patent Application Publication
(10) Pub. No.: US 2026/0209223 A1
(43) Pub. Date: Jul. 23, 2026

(54) FGFR3 INHIBITOR COMPOUNDS

(71) Applicant: TYRA BIOSCIENCES, INC., Carlsbad, CA (US)

(72) Inventor: Robert L. HUDKINS, Carlsbad, CA (US)

(21) Appl. No.: 19/140,426

(22) PCT Filed: Dec. 19, 2023
(86) PCT No.: PCT/US2023/084760

§ 371 Date: Jun. 18, 2025

Related U.S. Application Data
(60) Provisional application No. 63/476,019, filed on Dec. 19, 2022.

(51) Int. Cl. C07D 471/04; A61K 31/438; A61K 31/444; A61K 31/4545; A61K 31/4725; A61P 35/00

(52) U.S. Cl. CPC C07D 471/04; A61K 31/438; A61K 31/444; A61K 31/4545; A61K 31/4725; A61P 35/00

(57) ABSTRACT

The present invention provides compounds of formula (I) for use in the treatment of systemic sclerosis, fibrosis (e.g., pulmonary fibrosis), achondroplasia, thanatophoric dysplasia (e.g., type I), severe achondroplasia with developmental delay and acanthosis nigricans (SADDAN), muenke syndrome or cancer.

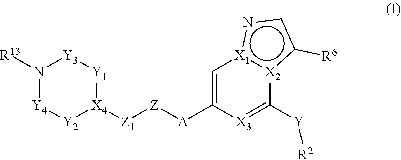

### CROSS REFERENCE TO RELATED APPLICATIONS

This application claims the benefit of U.S. Provisional Application No. 63/476,019, filed Dec. 19, 2022, the entirety of which is incorporated by reference herein.

### TECHNICAL FIELD

The disclosure is directed to compounds useful as FGFR inhibitors, as well as their methods of making and use.

### BACKGROUND

Fibroblast growth factor (FGF) has been recognized as an important mediator of many physiological processes, such as morphogenesis during development, fibrosis, and angiogenesis. The fibroblast growth factor receptor (FGFR) family consists of five members four of which (FGFR 1-4) are glycoproteins composed of extracellular immunoglobulin (Ig)-like domains, a hydrophobic transmembrane region and a cytoplasmic part containing a tyrosine kinase domain. FGF binding leads to FGFR dimerization, followed by receptor autophosphorylation and activation of downstream signaling pathways. Receptor activation is sufficient for the recruitment and activation of specific downstream signaling partners that participate in the regulation of diverse processes such as cell growth, cell metabolism and cell survival. Thus, the FGF/FGFR signaling pathway has pleiotropic effects on many biological processes critical to tumor cell proliferation, migration, invasion, and angiogenesis.

Therapeutic compounds that inhibit FGFR are needed.

### SUMMARY

Provided herein are compounds of the formula:

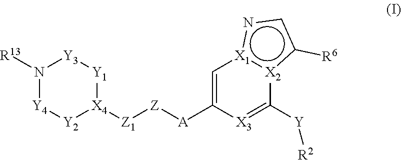

or a pharmaceutically acceptable salt thereof, wherein A, X₁, X₂, X₃, X₄, Y, Y₁, Y₂, Y₃, Y₄, Z, Z₁, R², R⁶ and R¹³ are as defined herein.

Provided herein are compounds of the formula:

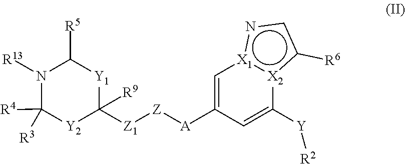

or a pharmaceutically acceptable salt thereof, wherein A, X₁, X₂, Y, Y₁, Y₂, Z, Z₁, R², R³, R⁴, R³, R⁶, R⁹ and R¹³ are as defined herein.

Provided herein are compounds of the formula:

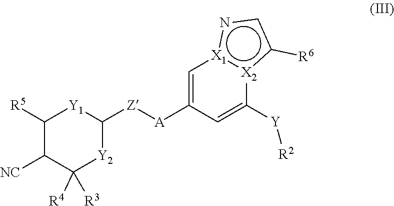

or a pharmaceutically acceptable salt thereof, wherein A, X₁, X₂, Y, Y₁, Y₂, Z′, R², R³, R⁴, R⁵ R⁶ and R¹³ are as defined herein.

Provided herein are pharmaceutical compositions comprising a compound of formula (I), (II), (IIA) or (III), or a pharmaceutically acceptable salt thereof, with one or more pharmaceutically acceptable carriers, diluents, or excipients.

Provided herein are methods of using the compounds of formula (I), (II), (IIA) or (III), or a pharmaceutically acceptable salt thereof, and pharmaceutical compositions thereof, to treat systemic sclerosis, fibrosis, pulmonary fibrosis, achondroplasia, thanatophoric dysplasia, severe achondroplasia with developmental delay and acanthosis nigricans (S ADD AN), muenke syndrome or proliferative disorders such as cancer, particularly to treat FGFR3-associated cancer. The methods include administering an effective amount of a compound of formula (I), (II), (IIA) or (III), or a pharmaceutically acceptable salt thereof, to a patient in need.

Provided herein, are compounds of formula (I), (II), (IIA) or (III), or a pharmaceutically acceptable salt thereof, for use in therapy. Further provided herein, are the compounds of formula (I), (II), (IIA) or (III), or a pharmaceutically acceptable salt thereof, for use in the treatment of systemic sclerosis, fibrosis, pulmonary fibrosis, achondroplasia, thanatophoric dysplasia, severe achondroplasia with developmental delay and acanthosis nigricans (SADDAN), muenke syndrome or cancer, particularly for use in the treatment of FGFR3-associated cancer. The use of compounds of formula (I), (II), (IIA) or (III), or a pharmaceutically acceptable salt thereof, in the manufacture of a medicament for treating systemic sclerosis, fibrosis, pulmonary fibrosis, achondroplasia, thanatophoric dysplasia, severe achondroplasia with developmental delay and acanthosis nigricans (SADDAN), muenke syndrome or cancer, particularly for use in the treatment of FGFR3-associated cancer, is also provided.

### DESCRIPTION

Provided herein are compounds believed to have clinical use for the treatment of systemic sclerosis, fibrosis, pulmonary fibrosis, achondroplasia, thanatophoric dysplasia, severe achondroplasia with developmental delay and acanthosis nigricans (SADDAN), muenke syndrome, proliferative disorders such as cancer and particularly for the treatment of FGFR3-associated cancer.

Certain compounds provided herein have superior FGFR3 potency compared to certain previously known FGFR inhibitors. Certain compounds provided herein have superior selectivity for FGFR3 over FGFR1 compared to certain previously known FGFR inhibitors, reducing potential dose limiting toxicity caused by inhibition of FGFR1 (e.g., hyperphosphatemia).

The compounds provided herein are of formula:

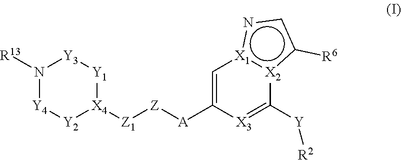

wherein
A is pyrazole, triazole, thiadiazole or oxadiazole, substituted with R¹ and R^{1A}, R¹ is hydrogen or C₁-C₃ alkyl;
R¹⁴ is hydrogen, halo, CN, or C₁-C₈ alkyl optionally substituted with one or more substituents independently selected from halo, OH, and OCH₃; X₁ and X₂ are independently selected from N and C, wherein when one of X₁ or X₂ is N the other is C;
X₃ is N or CH;
X₄ is N or C—R⁹;
Y is NH, O, S or a bond; Y₁ is a bond, CHR⁷, CH₂—CHR⁷, CHR⁷—CH₂, CF₂, CH₂—CF₂ or CF₂—CH₂;
Y₂ is a bond, CHR³, CH₂—CHR³, CHR³—CH₂, CF₂, CH₂—CF₂ or CF₂—CH₂;
Y₃ is CR⁴R⁵ or CF₂;
Y₄ is CR³R⁴, or CF₂;
Z is a bond, CHR^{9A}, CR⁴R^{4A}, CR⁴R^{4A}—CH₂, CH₂—CR⁴R^{4A}, cyclobutyl, cyclopentyl, cyclohexyl, bicyclo(1.1.1) pentane, bicyclo(2.1.1) hexane, azetidine, pyrrolidine or piperidine;
Z₁ is a bond when Z is a bond, CR⁴R^{4A}, CR⁴R^{4A}—CH₂, CH₂—CR⁴R^{4A}, cyclobutyl, cyclopentyl, cyclohexyl, bicyclo(1.1.1) pentane, bicyclo(2.1.1) hexane, azetidine, pyrrolidine or piperidine, or Z₁ is CH₂ or CH₂—CH₂ when Z is CHR^{9A};
Z₂ is a bond, C(O), SO₂ or —NR⁴C(O);
Z₃ is a bond, C(O), SO₂ or —NR⁴C(O);
R² is C₁-C₈ alkyl or R², wherein C₁-C₅ alkyl is optionally substituted with one or more substituents independently selected from halo, OH, CN, oxo, —OC₁-C₄ alkyl, —OC₃-C₅ cycloalkyl, —Z₂—R¹¹ and R¹⁰, wherein C₁-C₄ alky and C₃-C₅ cycloalkyl are optionally substituted with one or more substituents independently selected from halo, OH, OCH₃, methylamine, N,N-dimethylamine and CN;
R³ is hydrogen, F, OH, OCH₃, C₁-C₃ alkyl, cyclopropyl, or one R³ is fused with R⁵ or
R⁷ to form CH₂, CH₂—CH₂ or CH₂OCH₂;
R⁴ is hydrogen or C₁-C₃ alkyl;
R^{4A} is hydrogen, halo, OH, or C₁-C₃ alkyl;
R⁵ is hydrogen, F, OH, OCH₃, C₁-C₈ alkyl, cyclopropyl, or is fused with one R³ to form CH₂, CH₂—CH₂ or CH₂OCH₂; R⁶ is hydrogen, halo, C₁-C₈ alkyl, CN, 3-6 membered cycloalkyl, 4-6 membered heterocycloalkyl, 5-6 membered aryl or 5-6 membered heteroaryl, wherein 3-6 membered cycloalkyl, 4-6 membered heterocycloalkyl, 5-6 membered aryl and 5-6 membered heteroaryl are optionally substituted with one or more substituents independently selected from halo, methyl, halomethyl, OH or OCH₃ and wherein C₁-C₈ alkyl is optionally substituted with one or more substituents independently selected from halo, OH and OCH₃;
R⁷ is hydrogen, F, OH, OCH₃, C₁-C₃ alkyl or is fused with one R³ to form CH₂, CH₂—CH₂ or CH₂OCH₂;
R⁸ is 3-6 membered cycloalkyl, 4-6 membered heterocycloalkyl, 5-6 membered aryl or 5-6 membered heteroaryl, optionally fused or substituted with R^{8A},
R^{8A} is 3-6 membered cycloalkyl, 4-6 membered heterocycloalkyl, 5-6 membered aryl or 5-6 membered heteroaryl;
R⁹ is hydrogen, C₁-C₃ alkyl, or is fused with R^{9A} to form CH₂ or CH₂—CH₂;
R¹⁰ is 3-6 membered cycloalkyl, 4-6 membered heterocycloalkyl, 5-6 membered aryl or 5-6 membered heteroaryl, optionally fused or substituted with R^{8A};
R¹¹ is C₁-C₄ alkyl, NH₂, NHC₁-C₃ alkyl, NHC₃-C₅ cycloalkyl or N(C₁-C₃ alkyl)₂, wherein C₁-C₄ alkyl, C₁-C₃ alkyl and C₃-C₅ cycloalkyl are optionally substituted with one or more substituents independently selected from halo, OH, OCH₃, methylamine, N,N-dimethylamine and CN;
R¹² is C₁-C₄ alkyl, C₃-C₅ cycloalkyl, NH₂, NHC₁-C₃ alkyl, NHC₃-C₅ cycloalkyl or N(C₁-C₈ alkyl)₂, wherein C₁-C₄ alky, C₁-C₃ alkyl and C₃-C₅ cycloalkyl are optionally substituted with one or more substituents independently selected from halo, OH, OCH₃, methylamine, N,N-dimethylamine and CN;

### and R¹³ is

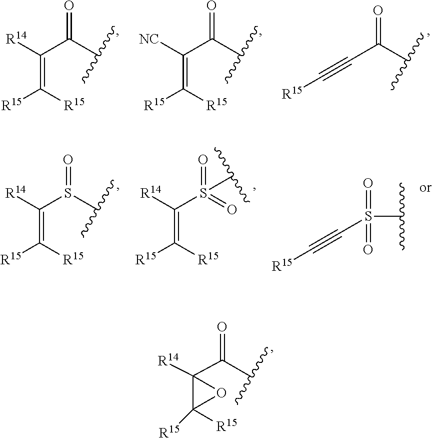

wherein each R¹⁴ and each R¹⁵ is independently H, optionally substituted C₁-C₆alkyl, or halogen; and
R₈, R¹⁰ and R^{8A} are optionally substituted with one or more substituents independently selected from halo, OH, CN, —OC₁-C₄ alkyl, —OC₃-C₅ cycloalkyl and —Z₃—R¹² wherein C₁-C₄ alky and C₃-C₅ cycloalkyl are optionally substituted with one or more substituents independently selected from halo, OH, OCH₃, methylamine, N, N-dimethylamine and CN; or a pharmaceutically acceptable salt thereof;
   and of formula:

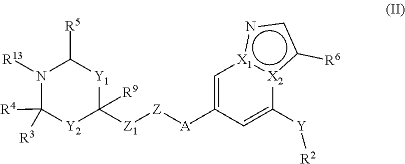

or a pharmaceutically acceptable salt thereof, and of formula:

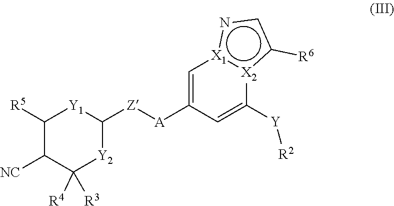

or a pharmaceutically acceptable salt thereof.

In formula (II), X₁ and X₂ are independently selected from N and C, wherein when one of X₁ or X₂ is N the other is C;
Z is a bond, CHR^{9A}, azetidine, pyrrolidine or piperidine; Z₁ is a bond when Z is a bond, azetidine, pyrrolidine or piperidine, or Z₁ is CH₂ or CH₂—CH₂ when Z is CHR^{9A}, and
R⁹ is hydrogen or is fused with ROA to form CH₂ or CH₂—CH₂.

In formula (III), X₁ and X₂ are independently selected from N and C, wherein when one of X₁ or X₂ is N the other is C; and
Z′ is a bond, azetidine, pyrrolidine or piperidine. In formula (II) and (III),
A is pyrazole, triazole, thiadiazole or oxadiazole, optionally substituted with R¹, R′ is C₁-C₃ alkyl;
Y is NH, O, or a bond; Y₁ is a bond, CHR⁷, CH₂—CHR⁷ or CHR⁷—CH₂, Y₂ is a bond, CH₂, CF₂, CHR³, CH₂—CHR³ or CHR³—CH₂; R² is C₁-C₅ alkyl or R⁸, wherein C₁-C₅ alkyl is optionally substituted with one or more substituents independently selected from OH, methoxy, halomethyl and R¹⁰;
R³ is hydrogen, C₁-C₈ alkyl, or one R³ is fused with R⁵ or R⁷ to form CH₂ or CH₂—CH₂;
R⁴ is hydrogen, or C₁-C₃ alkyl;
R⁵ is hydrogen, or is fused with one R³ to form CH₂ or CH₂—CH₂;
R⁶ is hydrogen, CH₂, CN, Cl or F;
R⁷ is hydrogen, or is fused with one R³ to form CH₂ or CH₂—CH₂,
R⁸ is 3-6 membered cycloalkyl, 5-6 membered heterocycloalkyl, 5-6 membered aryl or 5-6 membered heteroaryl, optionally fused with R^{8A};
R^{8A} is 3-6 membered cycloalkyl, 5-6 membered heterocycloalkyl, 5-6 membered aryl or 5-6 membered heteroaryl;
R¹⁰ is 3-6 membered cycloalkyl, 5-6 membered heterocycloalkyl, 5-6 membered aryl or 5-6 membered heteroaryl, optionally fused or substituted with R^{8A}, and
R⁸, R¹⁰ and R^{8A} are optionally substituted with one or more substituents independently selected from halogen, CN, methyl, halomethyl, methoxy, ethyl, ethoxy, methylamine, S(O)₂CH₃, C(O)NH₂, N,N-dimethylamine and C(O)N,N-dimethylamine.

In the compounds of formula (I), (II) or (III), X₁ can be C, and X₂ can be N; or X₁ can be N, and X₂ can be C.

In the compounds of formula (I), (II) or (III), X₁ can be C, and X₂ can be N, forming:

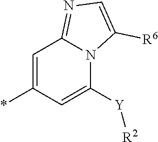

wherein * indicates the connection point to A in formula (I), (II) or (III).

In the compounds of formula (I), (II) or (III), X₁ can be N, and X₂ can be C, forming:

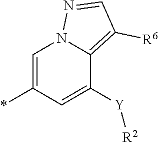

wherein * indicates the connection point to A in formula (I), (II) or (III).

In the compounds of formula (I), (II) or (III), R¹³ can be

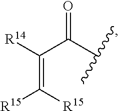

wherein each R¹⁴ and each R¹⁵ is independently H, optionally substituted C₁-C₆alkyl, or halogen.

In the compounds of formula (I), (II) or (III), R¹³ can be

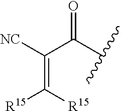

wherein each R¹⁵ is independently H, optionally substituted C₁-C₆alkyl, or halogen.

In the compounds of formula (I), (II) or (III), R¹³ can be

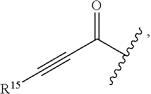

wherein R¹⁵ is H, optionally substituted C₁-C₆alkyl, or halogen.

In the compounds of formula (I), (II) or (III), R¹³ can be

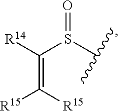

wherein each R¹⁴ and each R¹⁵ is independently H, optionally substituted C₁-C₆alkyl, or halogen.

In the compounds of formula (I), (II) or (III), R¹³ can be

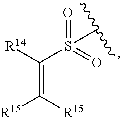

wherein each R¹⁴ and each R¹⁵ is independently H, optionally substituted C₁-C₆alkyl, or halogen.

In the compounds of formula (I), (II) or (III), R¹³ can be

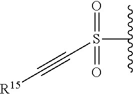

wherein R¹⁵ is H, optionally substituted C₁-C₆alkyl, or halogen.

In the compounds of formula (I), (II) or (III), R¹³ can be

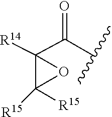

wherein each R¹⁴ and each R¹⁵ is independently H, optionally substituted C₁-C₆alkyl, or halogen.

The specific chemical naming conventions used herein are intended to be familiar to one of skill in the chemical arts. Some terms are defined specifically for additional clarity.

As used herein, the term “alkyl” refers to a hydrocarbon chain that may be a straight chain or branched chain, containing the indicated number of carbon atoms. For example, the term “C₁-C₅ alkyl” as used herein refers to saturated linear or branched-chain monovalent hydrocarbon radicals of one, two, three, four or five carbon atoms. Examples of C₁-C₅ alkyl include, but are not limited to, methyl, ethyl, 1-propyl, isopropyl, 1-butyl, isobutyl, sec-butyl, tert-butyl, 2-methyl-2-propyl, pentyl, and neopentyl. Examples of C₁-C₄ alkyl include, but are not limited to, methyl, ethyl, 1-propyl, isopropyl, 1-butyl, isobutyl, sec-butyl, tert-butyl, and 2-methyl-2-propyl. Examples of C₁-C₃ alkyl include, but are not limited to, methyl, ethyl, 1-propyl or isopropyl.

As used herein, the term “cycloalkyl” means a saturated cyclic hydrocarbon group containing the indicated number of carbon atoms. For example, the term “3-6 membered cycloalkyl” as used herein refers to a saturated cyclic hydrocarbon group having three, four, five or six carbon atoms. Examples of 3-6 membered cycloalkyl include, cyclopropyl, cyclobutyl, cyclopentyl and cyclohexyl.

As used herein, the term “heterocycloalkyl” means a saturated cyclic group containing the indicated number of atoms selected from C(O)₀₋₁, N, O and S(O)₀₋₂. For example, the term “5-6 membered heterocycloalkyl” as used herein refers to a saturated cyclic ring system having five or six ring atoms, one, two or three of which are selected from N, O and S(O)₀₋₂, the remainder being C(O)₀₋₁. Examples of 4-6 membered heterocycloalkyl groups include, but are not limited to, azetidinyl, pyrrolidinyl, piperidinyl, piperazinyl, pyrrolidinyl, pyrrolidin-2-onyl, dioxanyl, morpholinyl, oxetanyl, tetrahydrofuranyl, tetrahydrothiophenyl, tetrahydropyranyl, oxazolidinyl, isothiazolidinyl oxozolid-2-onyl and isothiazolid-2-onyl. Examples of 5-6 membered heterocycloalkyl groups include, but are not limited to, piperidinyl, piperazinyl, pyrrolidinyl, pyrrolidin-2-onyl, dioxanyl, morpholinyl, tetrahydrofuranyl, tetrahydrothiophenyl, tetrahydropyranyl, oxazolidinyl, isothiazolidinyl oxozolid-2-onyl and isothiazolid-2-onyl.

As used herein, the term “aryl” refers to an aromatic cyclic hydrocarbon group having the indicated number of carbon atoms. For example, the term “5-6 membered aryl” as used herein refers to an aromatic cyclic hydrocarbon group having five or six carbon atoms. Examples of 5-6 membered aryls include cyclopentadienyl and phenyl.

As used herein, the term “heteroaryl” refers to an aromatic cyclic group having the indicated number of atoms selected from C, N, O and S. For example, the term “5-6 membered heteroaryl” as used herein refers to an aromatic cyclic group having five or six ring atoms, one, two or three of which are selected from N, O and S, the remainder being C. Examples of 5-6 membered heteroaryls include, but are not limited to, pyridinyl, pyrazinyl, pyrimidinyl, pyridazinyl, furanyl, pyrrolyl, thiophenyl, imidazolyl, pyrazolyl, triazolyl, oxazolyl, isoxazolyl, thiazolyl, isothiazolyl, oxadiazolyl and thiadiazolyl. Examples of 6 membered heteroaryls include, but are not limited to, pyridinyl, pyrazinyl, pyrimidinyl and pyridazinyl.

As used herein the term “halogen” or “halo” refers to F (fluoro), Cl (chloro), Br (bromo) and I (iodo).

As used herein the term “halomethyl” refers to —CH₃, in which one or more hydrogen atoms is/are replaced with an independently selected halo.

As used herein the term “oxo” refers to the substitution of CH₂ with O to form C(O).

As used herein the term “N(C₁-C₃ alkyl)₂” allows the independent selection of each C₁-C₃alkyl substituent, for example, N may be substituted by methyl and ethyl.

As used herein the substituent —NR⁴C(O) is connected to R² through N.

Also provided is a compound of the formula:
wherein

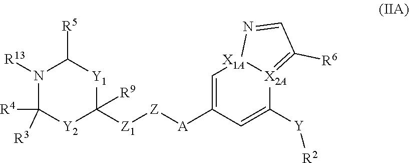

A is pyrazole, triazole, thiadiazole or oxadiazole, optionally substituted with R¹;
R¹ is C₁-C₈ alkyl;
X_{1A} and X_{2A} are independently selected from N and C, wherein when one of X_{1A} or X_{2A} is N the other is C;
Y is NH, O, or a bond; Y₁ is a bond, CHR⁷, CH₂—CHR⁷ or CHR⁷—CH₂;
Y₂ is a bond, CH₂, CF₂, CHR³, CH₂—CHR³ or CHR³—CH₂;
Z is a bond, CHR^{9A}, azetidine, pyrrolidine or piperidine; Z₁ is a bond when Z is a bond, azetidine, pyrrolidine or piperidine, or Z₁ is CH₂ or CH₂—CH, when Z is CHR^{9A};
R² is C₁-C₈ alkyl or R⁸, wherein C₁-C₈ alkyl is optionally substituted with one or more substituents independently selected from OH, methoxy, halomethyl and R¹⁰;
R³ is hydrogen, C₁-C₈ alkyl, or one R³ is fused with R⁵ or R⁷ to form CH₂ or CH₂—CH₂;
R⁴ is hydrogen, or C₁-C₃ alkyl;
R⁵ is hydrogen, or is fused with one R³ to form CH₂ or CH₂—CH₂;
R⁶ is hydrogen, CH₂, CN, Cl or F;
R⁷ is hydrogen, or is fused with one R³ to form CH₂ or CH₂—CH₂;
R⁸ is 3-6 membered cycloalkyl, 5-6 membered heterocycloalkyl, 5-6 membered aryl or 5-6 membered heteroaryl, optionally fused or substituted with R^{8A},
R^{8A} is 3-6 membered cycloalkyl, 5-6 membered heterocycloalkyl, 5-6 membered aryl or 5-6 membered heteroaryl;
R⁹ is hydrogen or is fused with ROA to form CH₂ or CH₂—CH₂; R¹⁰ is 3-6 membered cycloalkyl, 5-6 membered heterocycloalkyl, 5-6 membered aryl or 5-6 membered heteroaryl, optionally fused or substituted with R^{8A}, and R⁸, R¹⁰ and R^{8A} are optionally substituted with one or more substituents independently selected from halogen, CN, methyl, halomethyl, methoxy, ethyl, ethoxy, methylamine, S(O)₂CH₃, C(O)NH₂, N,N-dimethylamine and C(O) N,N-dimethylamine;

### and R¹³ is

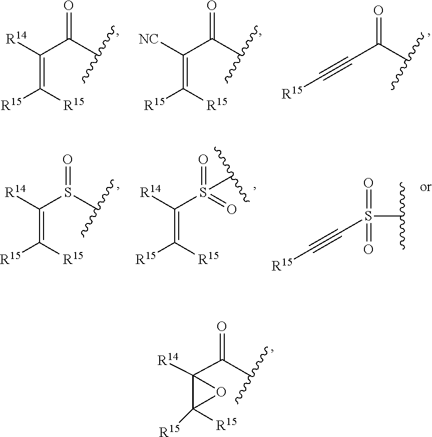

wherein each R¹⁴ and each R¹⁵ is independently H, optionally substituted C₁-C₆alkyl, or halogen;
or a pharmaceutically acceptable salt thereof.

In the compound of formula (IIA), X_{1A} can be C, and X_{2A} can be N, forming:

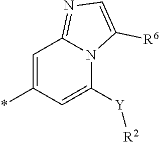

wherein * indicates the connection point to A in formula (IIA), forming a compound of the formula:

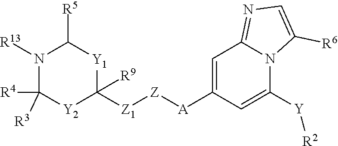

In the compound of formula (IIA), X_{1A} can be N, and X_{2A} can be C, forming:

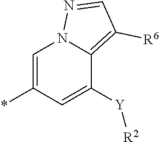

wherein * indicates the connection point to A in formula (IIA), forming a compound of the formula:

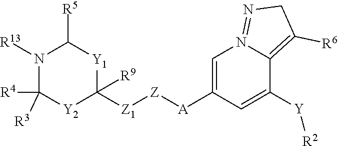

In the compounds of formula (I), A can be pyrazole, 1,2,3 triazole, 1,2,4 triazole, 1.2.3 thiadiazole, 1,2,4 thiadiazole, 1,2,5 thiadiazole, 1,3,4 thiadiazole, 1,2,3 oxadiazole, 1.2.4 oxadiazole, 1,2,5 oxadiazole, or 1,3,4 oxadiazole, substituted with R¹ and R^{1A}.

In the compounds of formula (I), A can be pyrazole, 1,2,3 triazole, or 1,2,4 triazole, substituted with R¹ and R^{1A}.

In the compounds of formula (I), A can be pyrazole, 1,2,3 triazole, or 1,2,4 triazole, substituted with R¹ and R^{1A}, wherein R^{1A} is hydrogen and R¹ is C₁-C₈ alkyl.

In the compounds of formula (I), A can be pyrazole, 1,2,3 triazole, or 1,2,4 triazole, substituted with R¹ and R^{1A}, wherein R^{1A} is hydrogen and R¹ is CH₃.

In the compounds of formula (II), (IIA) or (III), A can be pyrazole, 1,2,3 triazole, 1,2,4 triazole, 1,2,3 thiadiazole, 1,2,4 thiadiazole, 1,2,5 thiadiazole, 1,3,4 thiadiazole, 1,2,3 oxadiazole, 1,2,4 oxadiazole, 1,2,5 oxadiazole, or 1,3,4 oxadiazole, optionally substituted with R¹.

In the compounds of formula (II), (IIA) or (III), A can be pyrazole, 1,2,3 triazole, or 1,2,4 triazole, optionally substituted with R¹.

In the compounds of formula (II), (IIA) or (III), A can be pyrazole, 1,2,3 triazole, or 1,2,4 triazole, substituted with R¹.

In the compounds of formula (II), (IIA) or (III), A can be pyrazole, 1,2,3 triazole, or 1,2,4 triazole, substituted with CH₃.

In the compounds of formula (I), (II), (IIA) or (III), A can be:

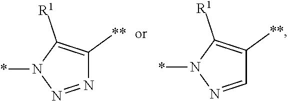

wherein * indicates the connection point to Z or Z′ and ** indicates the other connection point from A in formula (I), (II), (IIA) or (III); and R¹ can be C₁-C₃ alkyl.

In the compounds of formula (I), Z can be CHR^{9A}, cyclobutyl, azetidine, pyrrolidine or piperidine.

In the compounds of formula (I), Z can be a bond,

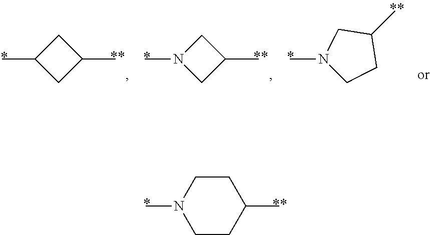

wherein * indicates the connection point to Z₁ and ** indicates the connection point to A in formula (I).

In the compounds of formula (I), Z can be a bond,

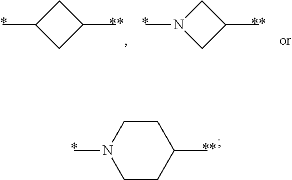

wherein * indicates the connection point to Z₁ and ** indicates the connection point to A in formula (I).

In the compounds of formula (II) or (IIA), Z can be CHR^{9A}, azetidine, pyrrolidine or piperidine.

In the compounds of formula (II) or (IIA), Z can be a bond,

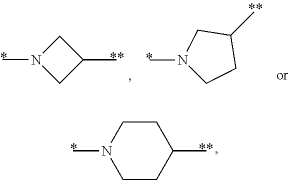

wherein * indicates the connection point to Z₁ and ** indicates the connection point to A in formula (II) or (IIA).

In the compounds of formula (II) or (IIA), Z can be:

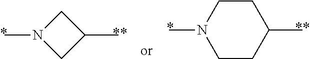

or wherein * indicates the connection point to Z₁ and ** indicates the connection point to A in formula (II) or (IIA).

In the compounds of formula (I), (II) or (IIA), Z can be CHR^{9A}, Z₁ can be selected from CH₂ or CH₂—CH₂, and R⁹ can be fused with R^{9A} to form CH₂ or CH₂—CH₂.

In the compounds of formula (I), (II) or (IIA), Z can be CHR^{9A}, Z₁ can be CH₂, and R⁹ can be fused with R^{9A} to form CH₂ or CH₂—CH₂.

In the compounds of formula (I), (II) or (IIA), Z can be CHR^{9A}, Z₁ can be CH₂—CH₂, and R⁹ can be fused with RA to form CH₂ or CH₂—CH₂.

In the compounds of formula (I), (II) or (IIA), Z can be CHR^{9A}, Z; can be selected from CH₂ or CH₂—CH₂, and R⁹ can be fused with R^{9A} to form CH₂.

In the compounds of formula (I), (II) or (IIA), Z can be CHR^{9A}, Z₁ can be selected from CH₂ or CH₂—CH₂, and R⁹ can be fused with R^{9A} to form CH₂—CH₂.

In the compounds of formula (I), (II) or (IIA), Z can be CHR^{9A}, Z₁ can be CH₂, and R⁹ can be fused with R^{9A} to form CH₂.

In the compounds of formula (I), (II) or (IIA), Z can be CHR^{9A}, Za can be CH₂—CH₂ and R⁹ can be fused with R^{9A} to form CH₂—CH₂.

In the compounds of formula (III), Z′ can be:

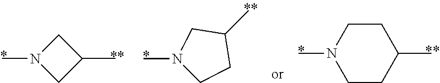

or wherein ** indicates the connection point to A and * indicates the other connection point from Z′ in formula (III).

In the compounds of formula (III), Z′ can be:

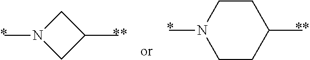

or wherein ** indicates the connection point to A and * indicates the other connection point from Z′ in formula (III).

In the compounds of formula (I), (II) or (IIA), Z can be a bond.

In the compounds of formula (III), Z′ can be a bond. In the compounds of formula (I), (II) or (IIA), Z₁ can be a bond.

In the compounds of formula (I), (II), (IIA) or (III), Y can be NH or O.

In the compounds of formula (I), (II), (IIA) or (III), Y can be O.

In the compounds of formula (I), Y₁ can be a bond, CHR⁷, CH₂—CHR⁷ or CHR⁷—CH₂, wherein R⁷ is selected from hydrogen, F, OH and CH₂; and Y₂ can a bond, CHR³, CH₂—CHR³ or CHR³—CH₂, wherein R³ is selected from hydrogen, F, OH and CH₃.

In the compounds of formula (I), Y₁ can be a bond or CHR⁷, wherein R⁷ is hydrogen, F, OH or CH₂; and Y₂ can a bond or CHR³, wherein R³ is hydrogen, F, OH or CH₂.

In the compounds of formula (I), Y₁ can be a bond, CHR⁷, CH₂—CHR⁷ or CHR⁷—CH₂, wherein R⁷ is hydrogen, F, OH or CH₂; and Ya can a bond, CHR³, CH₂—CHR³ or CHR³—CH₂, wherein R³ is hydrogen, F, OH or CH₂, forming:

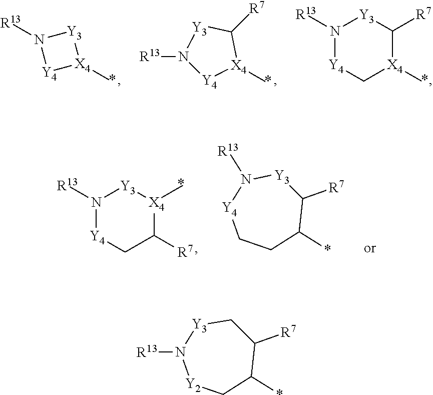

wherein * indicates the connection point to Z₁ in formula (I).

In the compounds of formula (I), Y₁ can be a bond or CHR⁷, wherein R⁷ is hydrogen, F, OH or CH₂; and Y₂ can a bond or CHR³, wherein R³ is hydrogen, F, OH or CH₂, forming:

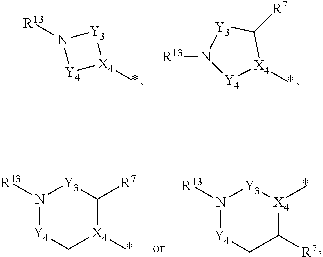

wherein * indicates the connection point to Z₁ in formula (I).

In the compounds of formula (II), (IIA) or (III), Y₁ can be a bond, CH₂, or CH₂—CH₂; and Y₂ can be a bond, CH₂, CF₂ or CH₂—CH₂, forming:

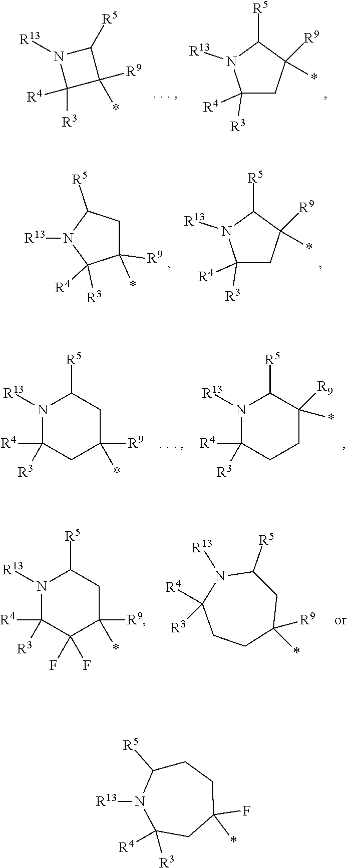

wherein * indicates the connection point to Z₁ in formula (II) or (IIA), or Z′ in formula (III).

In the compounds of formula (II), (IIA) or (III), Y₁ can be a bond, CH₂, or CH₂—CH₂; and Y₂ can be a bond, CH₂, or CF₂, forming:

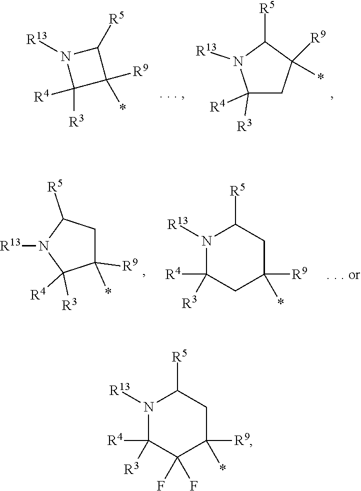

wherein * indicates the connection point to Z₁ in formula (II) or (IIA), or Z′ in formula (III).

In the compounds of formula (I), R^{1A} can be hydrogen, or C₁-C₃ alkyl optionally substituted with one or more substituents independently selected from halo, OH, and OCH₃.

In the compounds of formula (I), R^{1A} can be hydrogen or CH₃.

In the compounds of formula (I), R^{1A} can be hydrogen.

In the compounds of formula (I), (II), (IIA) or (III), R¹ can be methyl, ethyl or propyl.

In the compounds of formula (I), (II), (IIA) or (III), R¹ can be methyl.

In the compounds of formula (I), R² can be C₁-C₈ alkyl optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN.

In the compounds of formula (I), R² can be C₁-C₄ alkyl optionally substituted with one or more substituents independently selected from F, OH, CN, oxo, —OCH₃, —OC₃ cycloalkyl and R¹⁰.

In the compounds of formula (I), R² can be:

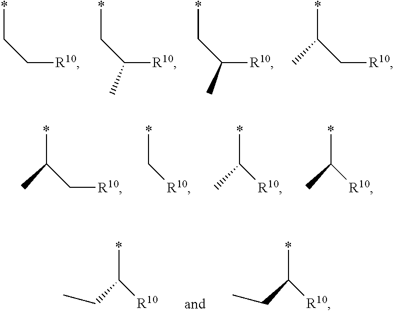

optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I).

In the compounds of formula (I), R² can be:

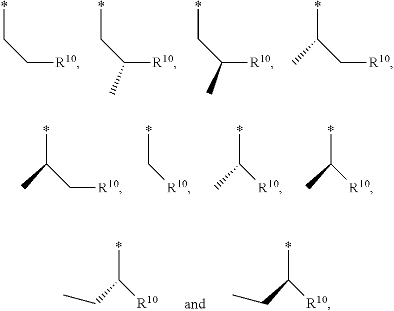

optionally substituted with one, two, three or four substituents independently selected from F, OH, CN, oxo, —OCH₃, —OC₃ cycloalkyl and R¹⁰, wherein * indicates the connection point to Y in formula (I).

In the compounds of formula (I), R² can be:

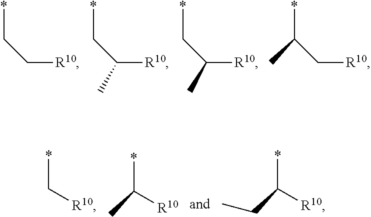

optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I).

In the compounds of formula (I), R² can be:

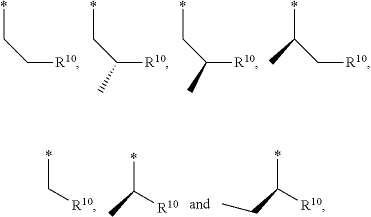

optionally substituted with one, two, three or four substituents independently selected from F, OH, CN, oxo, —OCH₃ and —OC₃ cycloalkyl and R¹⁰, wherein * indicates the connection point to Y in formula (I).

In the compounds of formula (II), (IIA) or (III), R² can be C₁-C₄ alkyl optionally substituted with one or more substituents independently selected from OH, methoxy, halomethyl and R¹⁰.

In the compounds of formula (II), (IIA) or (III), R² can be C₁-C₄ alkyl optionally substituted with one or two substituents independently selected from OH, methoxy, halomethyl and R¹⁰. In the compounds of formula (II), (IIA) or (III), R² can be C₁-C₃ alkyl optionally substituted with one or more substituents independently selected from OH, methoxy, halomethyl and R¹⁰.

In the compounds of formula (II), (IIA) or (III), R² can be C₁-C₃ alkyl optionally substituted with one, two or three substituents independently selected from OH, methoxy, halomethyl and R¹⁰.

In the compounds of formula (II), (IIA) or (III), R² can be C₁-C₂ alkyl optionally substituted with one or two substituents independently selected from OH, methoxy, halomethyl and R¹⁰.

In the compounds of formula (II), (IIA) or (III), R² can be:

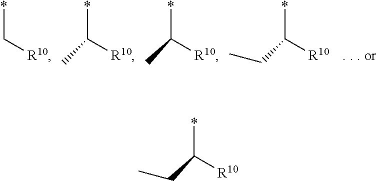

optionally substituted with one or two substituents independently selected from OH, CF₃ and methoxy, wherein * indicates the connection point to Y in formula (II), (IIA) or (III).

In the compounds of formula (II), (IIA) or (III), R² can be:

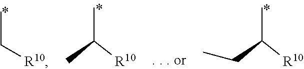

optionally substituted with one or two substituents independently selected from OH, CF₃ and methoxy, wherein * indicates the connection point to Y in formula (II), (IIA) or (III).

In the compounds of formula (II), (IIA) or (III), R² can be:

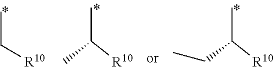

or optionally substituted with one or two substituents independently selected from OH, CF₃ and methoxy, wherein * indicates the connection point to Y in formula (II), (IIA) or (III).

In the compounds of formula (I), Y₃ can be CR⁴R³ or CF₂, wherein R⁴ is hydrogen or CH₃ and R⁵ is hydrogen, F, OH or CHs; and Ya is CR³R⁴ or CF; wherein R⁴ is hydrogen or CH₂, and R³ is hydrogen, F, OH or CH₃. In the compounds of formula (I), Y₃ can be CR⁴R⁵, wherein R⁴ is hydrogen and R⁵ is fused with one R³ to form CH₂, CH₂—CH₂ or CH₂OCH₂; and Y₄ is CR³R⁴ wherein R⁴ is hydrogen, and R³ is fused with R⁵ to form CH₂, CH₂—CH₂ or CH₂OCH₂.

In the compounds of formula (I), Y₃ can be CR⁴R⁵, wherein R⁴ is hydrogen and R⁵ is fused with one R³ to form CH₂, CH₂—CH₂ or CH₂OCH₂; and Ya is CR³R⁴ wherein R⁴ is hydrogen, and R³ is fused with R⁵ to form CH₂, CH₂—CH₂ or CH₂OCH₂, forming:

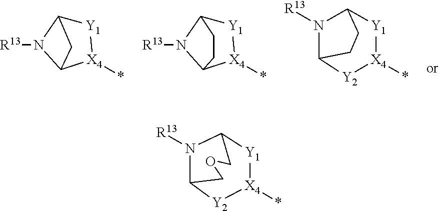

wherein * indicates the connection point to Z₁ in formula (I).

In the compounds of formula (I), X₄ can be N or C—R⁹ wherein R⁹ is hydrogen or CH₃.

In the compounds of formula (I), X₄ can be C—R⁹ wherein R⁹ is fused with R^{9A} to form CH₂ or CH₂—CH₂; and Z₁ is CH₂ or CH₂—CH₂.

In the compounds of formula (I), X₄ can be N or CH.

In the compounds of formula (II), (IIA) or (III), R³ can be hydrogen, C₁-C₂ alkyl, or fused with R⁵ to form CH₂ or CH—CH₂.

In the compounds of formula (II), (IIA) or (III), R³ can be hydrogen, C₁-C₂ alkyl, or fused with R₅ to form CH₂ or CH₂—CH₂.

In the compounds of formula (II), (IIA) or (III), R³ can be hydrogen or methyl.

In the compounds of formula (II), (IIA) or (III), R⁴ can be hydrogen or C₁-C₂ alkyl.

In the compounds of formula (II), (IIA) or (III), R⁴ can be hydrogen or methyl.

In the compounds of formula (II), (IIA) or (III), R³ and R⁴ can be hydrogen.

In the compounds of formula (II), (IIA) or (III), R₅ can be hydrogen.

In the compounds of formula (II), (IIA) or (III), R₅ can fuse with one R³ to form CH₂—CH₂.

In the compounds of formula (I), R⁶ can be CN, F, Cl, CH₃, CF₃ or cyclopropyl. In the compounds of formula (I), R⁶ can be CN, F or Cl.

In the compounds of formula (I), (II), (IIA) or (III), Re can be CN or Cl. In the compounds of formula (I), (II), (IIA) or (III), R⁶ can be CN.

In the compounds of formula (II), (IIA) or (III), R⁷ can be hydrogen.

In the compounds of formula (II), (IIA) or (III), R⁷ can fuse with one R³ to form CH₂.

In the compounds of formula (I), (II), (IIA) or (III), R⁸ can be 5-6 membered cycloalkyl, 5-6 membered heterocycloalkyl, 5-6 membered aryl or 5-6 membered heteroaryl, optionally fused or substituted with R^{8A}.

In the compounds of formula (I), (II), (IIA) or (III), R⁸ can be 5-6 membered cycloalkyl, or 5-6 membered heterocycloalkyl, optionally fused with R^{8A}.

In the compounds of formula (I), (II), (IIA) or (III), R⁸ can be cyclopentyl, cyclohexyl, piperidinyl, pyrrolidinyl, tetrahydrofuranyl, tetrahydropyranyl, phenyl or pyridinyl, optionally fused with R^{8A}.

In the compounds of formula (I), (II), (IIA) or (III), R⁸ can be cyclopentyl, cyclohexyl, piperidinyl, pyrrolidinyl, tetrahydrofuranyl or tetrahydropyranyl, fused with R^{8A}.

In the compounds of formula (I), (II), (IIA) or (III), R⁸ can be cyclopentyl, cyclohexyl, piperidinyl, pyrrolidinyl, tetrahydrofuranyl or tetrahydropyranyl, fused with R^{8A}, wherein R^{8A} can be phenyl or 6 membered heteroaryl.

In the compounds of formula (I), (II), (IIA) or (III), R⁹ can be hydrogen.

In the compounds of formula (I), (II), (IIA) or (III), R¹⁰ can be 3-6 membered cycloalkyl, 5-6 membered heterocycloalkyl, 5-6 membered aryl or 5-6 membered heteroaryl, optionally fused with R^{8A}.

In the compounds of formula (I), (II), (IIA) or (III), R¹⁰ can be 3-6 membered cycloalkyl, 5-6 membered heterocycloalkyl, phenyl or 5-6 membered heteroaryl, optionally fused with R^{8A}.

In the compounds of formula (I), (II), (IIA) or (III), R¹⁰ can be cyclopropyl, cyclobutyl, cyclopentyl, cyclohexyl, piperidinyl, piperazinyl, pyrrolidinyl, pyrrolidin-2-onyl, dioxanyl, morpholinyl, tetrahydrofuranyl, tetrahydrothiophenyl, tetrahydropyranyl, oxazolidinyl, isothiazolidinyl, oxozolid-2-onyl, isothiazolid-2-onyl, phenyl, pyridinyl, pyrazinyl, pyrimidinyl, pyridazinyl, furanyl, pyrrolyl, thiophenyl, imidazolyl, pyrazolyl, triazolyl, oxazolyl, isoxazolyl, thiazolyl, isothiazolyl, oxadiazolyl or thiadiazolyl, optionally fused with R^{8A}.

In the compounds of formula (I), (II), (IIA) or (III), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, pyrazolyl, triazolyl, oxazolyl, isoxazolyl, thiazolyl, isothiazolyl, oxadiazolyl or thiadiazolyl, optionally fused with R^{8A}.

In the compounds of formula (I), (II), (IIA) or (III), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, oxazolyl, isoxazolyl, thiazolyl, isothiazolyl, oxadiazolyl or thiadiazolyl, optionally fused with R^{8A}.

In the compounds of formula (I), (II), (IIA) or (III), R¹⁰ can be cyclopropyl, cyclobutyl, phenyl, pyridinyl, oxazolyl, isoxazolyl, thiazolyl or isothiazolyl, optionally fused with R^{8A}.

In the compounds of formula (I), (II), (IIA) or (III), R¹⁰ can be cyclopropyl, cyclobutyl, cyclopentyl, cyclohexyl, piperidinyl, piperazinyl, pyrrolidinyl, pyrrolidin-2-onyl, dioxanyl, morpholinyl, tetrahydrofuranyl, tetrahydrothiophenyl, tetrahydropyranyl, oxazolidinyl, isothiazolidinyl, oxozolid-2-onyl, isothiazolid-2-onyl, phenyl, pyridinyl, pyrazinyl, pyrimidinyl, pyridazinyl, furanyl, pyrrolyl, thiophenyl, imidazolyl, pyrazolyl, triazolyl, oxazolyl, isoxazolyl, thiazolyl, isothiazolyl, oxadiazolyl or thiadiazolyl.

In the compounds of formula (I), (II), (IIA) or (III), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, pyrazolyl, triazolyl, oxazolyl, isoxazolyl, thiazolyl, isothiazolyl, oxadiazolyl or thiadiazolyl.

In the compounds of formula (I), (II), (IIA) or (III), R¹⁰ can be cyclopropyl, cyclobutyl, cyclopentyl, cyclohexyl, tetrahydrofuranyl, tetrahydropyranyl, phenyl, pyridinyl, pyrazinyl, pyrimidinyl, pyridazinyl, oxazolyl, isoxazolyl, thiazolyl, isothiazolyl, oxadiazolyl or thiadiazolyl.

In the compounds of formula (I), (II), (IIA) or (III), R¹⁰ can be cyclopropyl, cyclobutyl, phenyl, pyridinyl, oxazolyl, isoxazolyl, thiazolyl or isothiazolyl. In the compounds of formula (II), (IIA) or (III), R¹⁰ can be 5-6 membered cycloalkyl, 5-6 membered heterocycloalkyl, phenyl or 5-6 membered heteroaryl, fused with R^{8A}.

In the compounds of formula (I), (II), (IIA) or (III), R¹⁰ can be cyclopentyl, cyclohexyl, tetrahydrofuranyl, tetrahydropyranyl, phenyl, pyridinyl, pyrazinyl, pyrimidinyl, pyridazinyl, pyrazolyl, triazolyl, oxazolyl, isoxazolyl, thiazolyl, isothiazolyl, oxadiazolyl or thiadiazolyl, fused with R^{8A}.

In the compounds of formula (I), (II), (IIA) or (III), R¹⁰ can be cyclopentyl, cyclohexyl, piperidinyl, pyrrolidinyl, tetrahydrofuranyl, tetrahydropyranyl, phenyl or pyridinyl, fused with R^{8A}.

In the compounds of formula (I), (II), (IIA) or (III), R¹⁰ can be cyclopentyl, cyclohexyl, piperidinyl, pyrrolidinyl, tetrahydrofuranyl, tetrahydropyranyl, phenyl or pyridinyl, fused with R^{8A}.

In the compounds of formula (I, (II), (IIA) or (III), RIC can be cyclopentyl, cyclohexyl, piperidinyl, pyrrolidinyl, tetrahydrofuranyl, tetrahydropyranyl, phenyl or pyridinyl, fused with R^{8A}.

In the compounds of formula (I), (II), (IIA) or (III), R¹⁰ can be cyclopentyl, cyclohexyl, phenyl or pyridinyl, fused with R^{8A}.

In the compounds of formula (I), (II), (IIA) or (III), R¹⁰ can be phenyl or pyridinyl, fused with R^{8A} wherein R^{8A} can be 5-6 membered heterocycloalkyl or 5-6 membered heteroaryl.

In the compounds of formula (I), (II), (IIA) or (III), R¹⁰ can be phenyl or pyridinyl, fused with R^{8A} wherein R^{8A} can be pyrrolidinyl, pyrrolidin-2-onyl, dioxanyl, tetrahydrofuranyl, tetrahydrothiophenyl, tetrahydropyranyl, oxazolidinyl, isothiazolidinyl, oxozolid-2-onyl, isothiazolid-2-onyl furanyl, pyrrolyl, thiophenyl, imidazolyl, pyrazolyl, triazolyl, oxazolyl, isoxazolyl, thiazolyl, isothiazolyl, oxadiazolyl or thiadiazolyl.

In the compounds of formula (I), (II), (IIA) or (III), R¹⁰ can be phenyl or pyridinyl, fused with R^{8A} wherein R^{8A} can be tetrahydrofuranyl, tetrahydrothiophenyl, tetrahydropyranyl, oxazolidinyl, isothiazolidinyl, oxozolid-2-onyl, isothiazolid-2-onyl, furanyl, pyrrolyl, thiophenyl, imidazolyl, pyrazolyl, triazolyl, oxazolyl, isoxazolyl, thiazolyl, isothiazolyl, oxadiazolyl or thiadiazolyl. In the compounds of formula (I), (II), (IIA) or (III), R¹⁰ can be phenyl or pyridinyl, fused with RSA wherein R^{8A} can be tetrahydrofuranyl, tetrahydrothiophenyl, tetrahydropyranyl, oxazolidinyl, isothiazolidinyl, oxozolid-2-only or isothiazolid-2-onyl.

In the compounds of formula (I), (II) or (IIA), where both Z and Z₁ are a bond, together they form a single bond.

In the compounds of formula (I), Z can be CHR^{9A}, Z; can be CH₂, X₄ can be C—R⁹, and R⁹ can be fused with R^{9A} to form CH₂, forming:

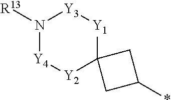

wherein * indicates the connection point to A.

In the compounds of formula (II) or (IIA), Z can be CHR^{9A}, Z₁ can be CH₂, and R⁹ can be fused with R^{9A} to form CH₂, forming:

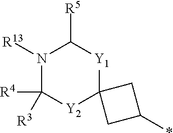

wherein * indicates the connection point to A.

In the compounds of formula (I), (II), (IIA) or (III), R⁵ can be fused with one R³ to form CH₂—CH₂, for example forming:

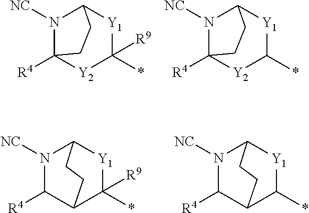

wherein * indicates the connection point to Z₁ in formula (I) or Z′ in formula (III).

In the compounds of formula (I), (II), (IIA) or (III), R⁸ can be cyclopentyl, fused with R^{8A}, wherein R^{8A} can be pyridinyl, for example forming:

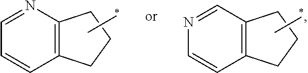

wherein * indicates the connection point to Y.

In the compounds of formula (I), A can be pyrazole, 1,2,3 triazole, or 1,2,4 triazole, substituted with R¹ and R^{1A}, and Y can be NH or O.

In the compounds of formula (I), A can be pyrazole, 1,2,3 triazole, or 1,2,4 triazole, substituted with R¹ and R^{1A}, wherein R^{1A} is hydrogen and R¹ is C₁-C₈ alkyl; and Y can be NH or O.

In the compounds of formula (II), (IIA) or (III), A can be pyrazole, 1,2,3 triazole, or 1,2,4 triazole, substituted with R¹; and Y can be NH or O.

In the compounds of formula (I), (II), (IIA) or (III), A can be:

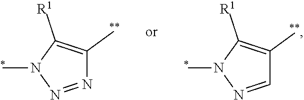

wherein * indicates the connection point to Z or Z′ and ** indicates the other connection point from A in formula (I), (II), (IIA) or (III); R¹ can be C₁-C₃ alkyl; and Y can be NH or O.

In the compounds of formula (I), A can be pyrazole, 1,2,3 triazole, or 1,2,4 triazole, substituted with R¹ and R^{1A}, and Y can be O.

In the compounds of formula (I), A can be pyrazole, 1,2,3 triazole, or 1,2,4 triazole, substituted with R¹ and R^{1A}, wherein R^{1A} is hydrogen and R¹ is C₁-C₈ alkyl; and Y can be O.

In the compounds of formula (II), (IIA) or (III), A can be pyrazole, 1,2,3 triazole, or 1,2,4 triazole, substituted with R′, and Y can be O. In the compounds of formula (I), (II), (IIA) or (III), A can be:

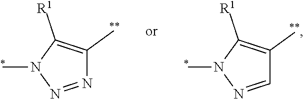

wherein * indicates the connection point to Z or Z′ and ** indicates the other connection point from A in formula (I), (II), (IIA) or (III); R′ can be C₁-C₃ alkyl; and Y can be O.

In the compounds of formula (I), A can be pyrazole, 1,2,3 triazole, or 1,2,4 triazole, substituted with R¹ and R^{1A}, and R⁶ can be CN, F, Cl, CH₃, CF₃ or cyclopropyl.

In the compounds of formula (I), A can be pyrazole, 1,2,3 triazole, or 1,2,4 triazole, substituted with R¹ and R^{1A}, wherein R^{1A} is hydrogen and R¹ is C₁-C₈ alkyl; and R⁶ can be CN, F, Cl or CF₃.

In the compounds of formula (I), A can be:

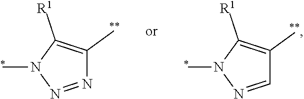

wherein * indicates the connection point to Z or Z′ and ** indicates the other connection point from A in formula (I); and R⁶ can be CN, F, Cl, CH₃, CF₃ or cyclopropyl.

In the compounds of formula (I), A can be:

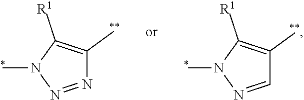

wherein * indicates the connection point to Z or Z′ and ** indicates the other connection point from A in formula (I); and R⁶ can be CN, F, Cl or CF₃. In the compounds of formula (II), (IIA) or (III), A can be pyrazole, 1,2,3 triazole, or 1,2,4 triazole, substituted with R¹; and R⁹ can be CN or Cl.

In the compounds of formula (I), (II), (IIA) or (III), A can be:

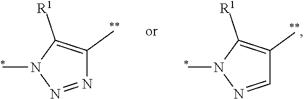

wherein * indicates the connection point to Z or Z′ and ** indicates the other connection point from A in formula (I), (II), (IIA) or (III); R¹ can be C₁-C₃ alkyl; and R⁹ can be CN or Cl.

In the compounds of formula (I), A can be pyrazole, 1,2,3 triazole, or 1,2,4 triazole, substituted with R¹ and RIA; R⁶ can be CN, F, Cl, CH₃, CF₃ or cyclopropyl; and Y can be NH or O.

In the compounds of formula (I), A can be pyrazole, 1,2,3 triazole, or 1,2,4 triazole, substituted with R¹ and R^{1A}, wherein R^{1A} is hydrogen and R¹ is C₁-C₈ alkyl; R⁶ can be CN, F, Cl or CF₃; and Y can be NH or O.

In the compounds of formula (I), A can be:

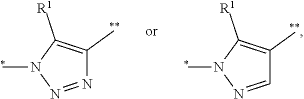

wherein * indicates the connection point to Z or Z′ and ** indicates the other connection point from A in formula (I); R⁶ can be CN, F, Cl, CH₃, CF₃ or cyclopropyl; and Y can be NH or O.

In the compounds of formula (I), A can be:

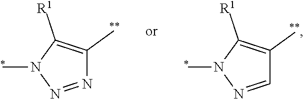

wherein * indicates the connection point to Z or Z′ and ** indicates the other connection point from A in formula (I); Re can be CN, F, Cl or CF₃; and Y can be NH or O.

In the compounds of formula (II), (IIA) or (III), A can be pyrazole, 1,2,3 triazole, or 1,2,4 triazole, substituted with R¹, R⁶ can be CN or Cl; and Y can be NH or O.

In the compounds of formula (I), (II), (IIA) or (III), A can be:

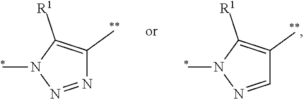

wherein * indicates the connection point to Z or Z′ and ** indicates the other connection point from A in formula (I), (II), (IIA) or (III); R¹ can be C₁-C₃ alkyl; R⁶ can be CN or Cl; and Y can be NH or O.

In the compounds of formula (I), A can be pyrazole, 1,2,3 triazole, or 1,2,4 triazole, substituted with R¹ and R^{1A}, R⁶ can be CN, F, CL, CH₂, CF₃ or cyclopropyl; and Y can be O.

In the compounds of formula (I), A can be pyrazole, 1,2,3 triazole, or 1,2,4 triazole, substituted with R¹ and R^{1A}, wherein R^{1A} is hydrogen and R¹ is C₁-C₈ alkyl; R⁶ can be CN, F, Cl or CF₃; and Y can be O.

In the compounds of formula (I), A can be:

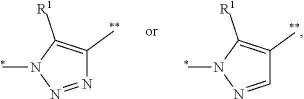

wherein * indicates the connection point to Z or Z′ and ** indicates the other connection point from A in formula (I); R⁶ can be CN, F, Cl, CH₂, CF₃ or cyclopropyl; and Y can be O.

In the compounds of formula (I), A can be:

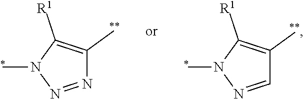

wherein * indicates the connection point to Z or Z′ and ** indicates the other connection point from A in formula (I); R⁶ can be CN, F, Cl or CF₃; and Y can be O.

In the compounds of formula (II), (IIA) or (III), A can be pyrazole, 1,2,3 triazole, or 1,2,4 triazole, substituted with R¹, R⁶ can be CN or Cl; and Y can be O.

In the compounds of formula (I), (II), (IIA) or (III), A can be:

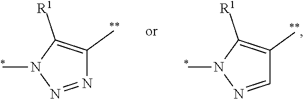

wherein * indicates the connection point to Z or Z′ and ** indicates the other connection point from A in formula (I), (II), (IIA) or (III); R¹ can be C₁-C₃ alkyl; R⁶ can be CN or Cl; and Y can be O.

In the compounds of formula (I), A can be pyrazole, 1,2,3 triazole, or 1,2,4 triazole, substituted with R¹ and R¹⁴, R⁶ can be CN, F, Cl, CH₂, CF₃ or cyclopropyl; Y can be NH or O; and R² can be C₁-C₈ alkyl optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN.

In the compounds of formula (I), A can be pyrazole, 1,2,3 triazole, or 1,2,4 triazole, substituted with R¹ and R^{1A}, R⁶ can be CN, F, Cl, CH₂, CF₃ or cyclopropyl, Y can be NH or O; and R² can be C₁-C₄ alkyl optionally substituted with one or more substituents independently selected from F, OH, CN, oxo, —OCH₃, —OC₃ cycloalkyl and R¹⁰.

In the compounds of formula (I), A can be pyrazole, 1,2,3 triazole, or 1,2,4 triazole, substituted with R¹ and R^{1A}, Re can be CN, F, Cl or CF₃; Y can be NH or O; and R² can be:

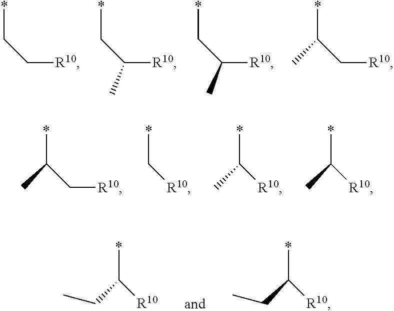

optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I).

In the compounds of formula (I), A can be pyrazole, 1,2,3 triazole, or 1,2,4 triazole, substituted with R¹ and RIA; R⁶ can CN, F, Cl or CF₃; Y can be NH or O; and R² can be:

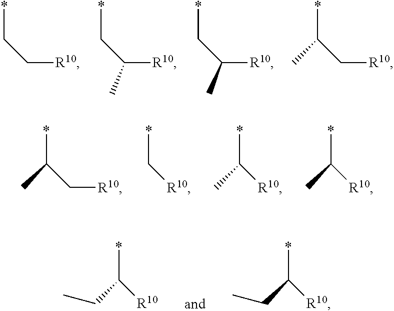

optionally substituted with one, two, three or four substituents independently selected from F, OH, CN, oxo, —OCH₃, —OC₃ cycloalkyl and R¹⁰, wherein * indicates the connection point to Y in formula (I).

In the compounds of formula (I), A can be pyrazole, 1,2,3 triazole, or 1,2,4 triazole, substituted with R¹ and RIA; R⁶ can be CN, F, Cl or CF₃; Y can be NH or O; and R² can be:

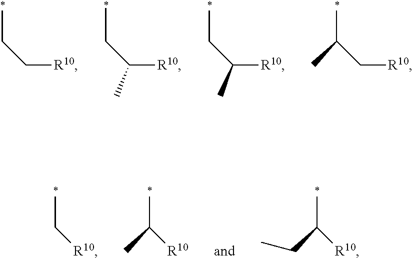

optionally substituted with one, two, three or four substituents independently selected from halo, OH, CN, oxo, —OC₁-C₄ alkyl, —OC₃-C₅ cycloalkyl, —Z₂—R¹¹ and R¹, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I).

In the compounds of formula (I), A can be pyrazole, 1,2,3 triazole, or 1,2,4 triazole, substituted with R¹ and R^{1A}, R⁵ can CN, F, Cl or CF₃; Y can be NH or O, and R² can be:

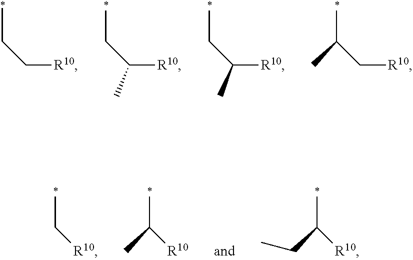

optionally substituted with one, two, three or four substituents independently selected from F, OH, CN, oxo, —OCH₃, —OC₃ cycloalkyl and R¹⁰, wherein * indicates the connection point to Y in formula (I).

In the compounds of formula (I), A can be:

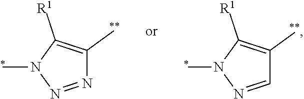

wherein * indicates the connection point to Z or Z′ and ** indicates the other connection point from A in formula (I); Re can be CN, F, Cl, CH₂, CF₃ or cyclopropyl; Y can be NH or O; and R² can be C₁-C₃ alkyl optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN.

In the compounds of formula (I), A can be:

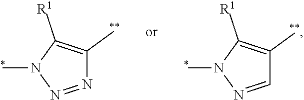

wherein * indicates the connection point to Z or Z′ and ** indicates the other connection point from A in formula (I); Re can be CN, F, Cl, CH₂, CF₃ or cyclopropyl; Y can be NH or O; and R² can be C₁-C₄ alkyl optionally substituted with one or more substituents independently selected from F, OH, CN, oxo, —OCH₃, —OC₃ cycloalkyl and R¹⁰.

In the compounds of formula (I), A can be:

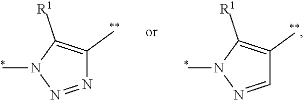

wherein * indicates the connection point to Z or Z′ and ** indicates the other connection point from A in formula (I); Re can be CN, F, Cl or CF₃; Y can be NH or O; and R² can be:

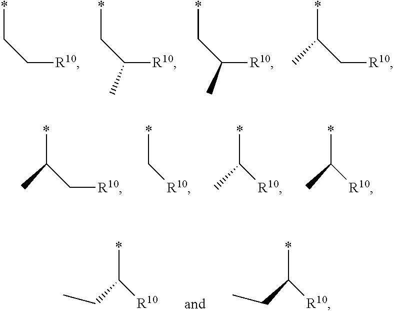

optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I).

In the compounds of formula (I), A can be:

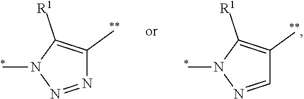

wherein * indicates the connection point to Z or Z′ and ** indicates the other connection point from A in formula (I); Re can CN, F, Cl or CF₃; Y can be NH or O; and R² can be:

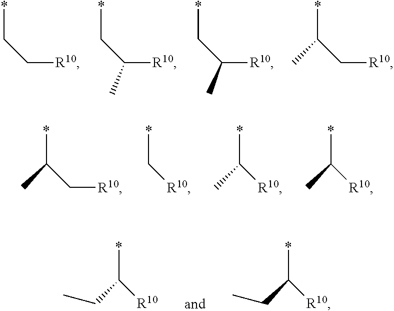

optionally substituted with one, two, three or four substituents independently selected from F, OH, CN, oxo, —OCH₃, —OC₃ cycloalkyl and R¹⁰, wherein * indicates the connection point to Y in formula (I).

In the compounds of formula (I), A can be:

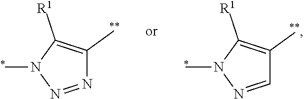

wherein * indicates the connection point to Z or Z′ and ** indicates the other connection point from A in formula (I); Re can be CN, F, Cl or CF₃; Y can be NH or O; and R² can be:

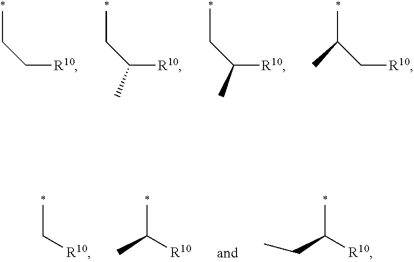

optionally substituted with one, two, three or four substituents independently selected from halo, OH, CN, oxo, —OC₁-C₄ alkyl, —OC₃-C₅ cycloalkyl, —Z₂—R¹¹ and R¹, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I).

In the compounds of formula (I), A can be:

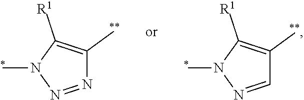

wherein * indicates the connection point to Z or Z′ and ** indicates the other connection point from A in formula (I); R⁶ can CN, F, Cl or CF₃; Y can be NH or O; and R² can be:

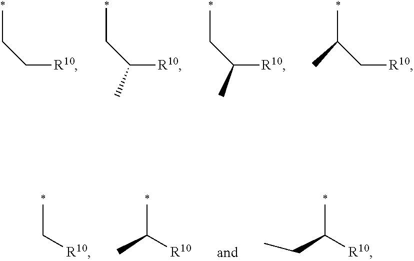

optionally substituted with one, two, three or four substituents independently selected from F, OH, CN, oxo, —OCH₃, —OC₃ cycloalkyl and R¹⁰, wherein * indicates the connection point to Y in formula (I).

In the compounds of formula (II), (IIA) or (III), A can be pyrazole, 1,2,3 triazole, or 1,2,4 triazole, substituted with R¹; Y can be O; R⁶ can be CN or Cl; and R² can be C₁-C₃ alkyl optionally substituted with one, two or three substituents independently selected from OH, methoxy, halomethyl and R¹⁰.

In the compounds of formula (II), (IIA) or (III), A can be:

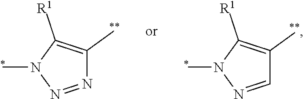

wherein * indicates the connection point to Z or Z′ and ** indicates the other connection point from A in formula (II), (IIA) or (III); Y can be O; R⁵ can be CN or Cl; and R² can be C₁-C₈ alkyl optionally substituted with one, two or three substituents independently selected from OH, methoxy, halomethyl and R¹⁰.

In the compounds of formula (II), (IIA) or (III), A can be pyrazole, 1,2,3 triazole, or 1,2,4 triazole, substituted with R¹; Y can be O); R⁶ can be CN or Cl; and R² can be:

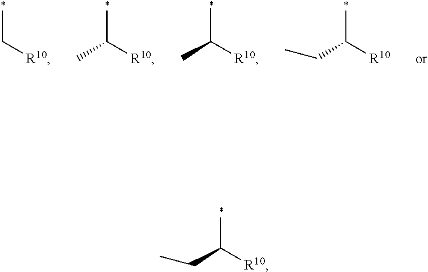

optionally substituted with one or two substituents independently selected from OH, CF₃ and methoxy, wherein * indicates the connection point to Y in formula (II), (IIA) or (III). In the compounds of formula (II), (IIA) or (III), A can be:

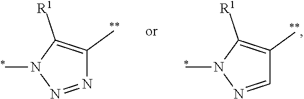

wherein * indicates the connection point to Z or Z′ and ** indicates the other connection point from A in formula (II), (IIA) or (III); Y can be O; R⁶ can be CN or Cl; and R² can be:

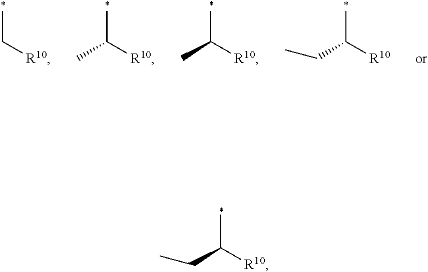

optionally substituted with one or two substituents independently selected from OH, CF₃ and methoxy, wherein * indicates the connection point to Y in formula (II), (IIA) or (III).

In the compounds of formula (I), A can be pyrazole, 1,2,3 triazole, or 1,2,4 triazole, substituted with R¹ and RIA; R⁶ can be CN, F, Cl, CH₂, CF₃ or cyclopropyl; Y can be NH or O; R² can be C₁-C₈ alkyl optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN; and Z can be a bond,

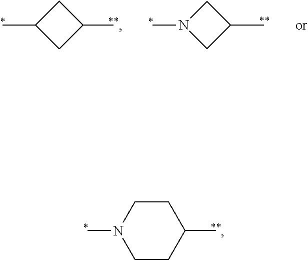

wherein * indicates the connection point to Z₁ and ** indicates the connection point to A in formula (I).

In the compounds of formula (I), A can be pyrazole, 1,2,3 triazole, or 1,2,4 triazole, substituted with R¹ and R^{1A}, R⁶ can be CN, F, Cl, CH₂, CF₃ or cyclopropyl; Y can be NH or O, R² can be C₁-C₄ alkyl optionally substituted with one or more substituents independently selected from F, OH, CN, oxo, —OCH₃, —OC₃ cycloalkyl and R¹⁰; and Z can be a bond,

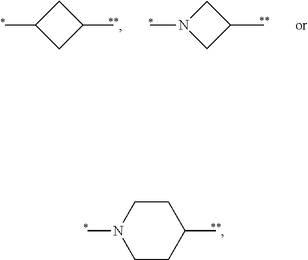

or wherein * indicates the connection point to Z₁ and ** indicates the connection point to A in formula (I).

In the compounds of formula (I), A can be pyrazole, 1,2,3 triazole, or 1,2,4 triazole, substituted with R¹ and R^{1A}, R⁶ can be CN, F, Cl or CF₃; Y can be NH or O; and R² can be:

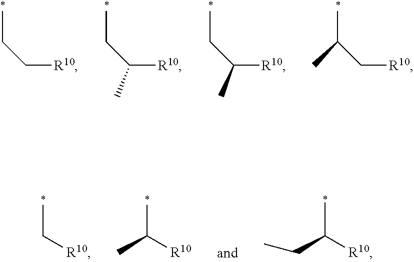

optionally substituted with one, two, three or four substituents independently selected from halo, OH, CN, oxo, ~OC₁-C₄ alkyl, —OC₃-C₅ cycloalkyl, —Z2-R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I); and Z can be a bond,

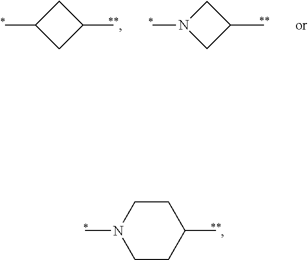

wherein * indicates the connection point to Z₁ and ** indicates the connection point to A in formula (I).

In the compounds of formula (I), A can be pyrazole, 1,2,3 triazole, or 1,2,4 triazole, substituted with R¹ and R^{1A}, R⁶ can CN, F, Cl or CF₃; Y can be NH or O; and R² can be:

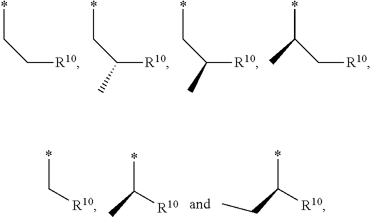

optionally substituted with one, two, three or four substituents independently selected from F, OH, CN, oxo, —OCH₃, —OC₃ cycloalkyl and R¹⁰, wherein * indicates the connection point to Y in formula (I); and Z can be a bond,

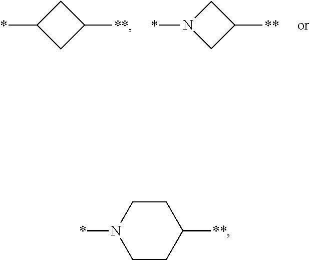

wherein * indicates the connection point to Z₁ and ** indicates the connection point to A in formula (I).

In the compounds of formula (I), A can be:

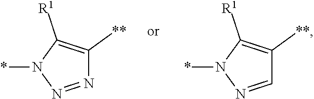

wherein * indicates the connection point to Z or Z′ and ** indicates the other connection point from A in formula (I); R⁶ can be CN, F, Cl, CH₂, CF₃ or cyclopropyl; Y can be NH or O; R² can be C₁-C₃ alkyl optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN; and Z can be a bond,

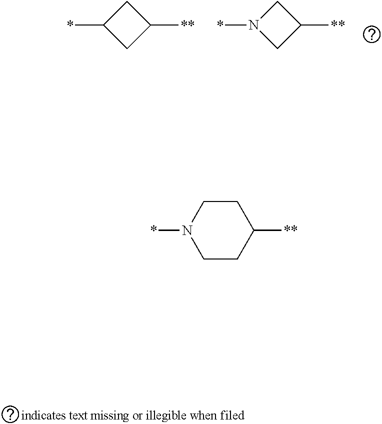

or wherein * indicates the connection point to Z₁ and ** indicates the connection point to A in formula (I).

In the compounds of formula (I), A can be:

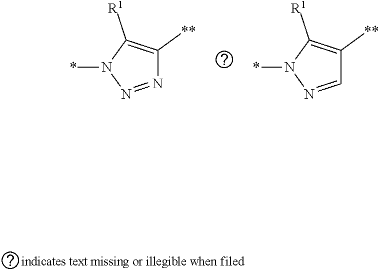

or wherein * indicates the connection point to Z or Z′ and ** indicates the other connection point from A in formula (I); R⁶ can be CN, F, Cl, CH₂, CF₃ or cyclopropyl; Y can be NH or O; R² can be C₁-C₄ alkyl optionally substituted with one or more substituents independently selected from F, OH, CN, oxo, —OCH₃, —OC₃ cycloalkyl and R¹⁰; and Z can be a bond,

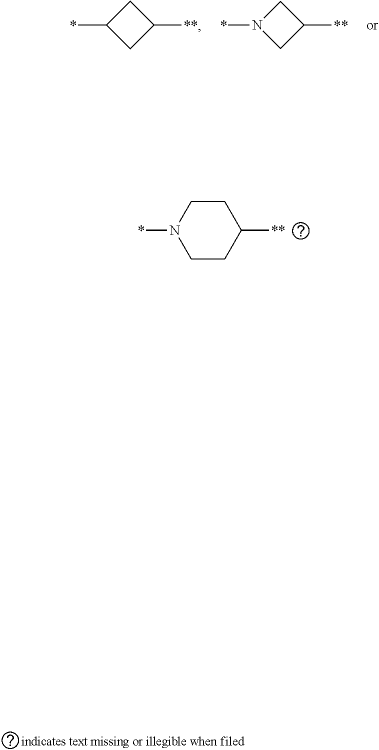

wherein * indicates the connection point to Z₁ and ** indicates the connection point to A in formula (I).

In the compounds of formula (I), A can be:

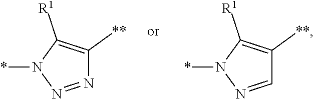

wherein * indicates the connection point to Z or Z′ and ** indicates the other connection point from A in formula (I); R⁶ can be CN, F, Cl or CF₃; Y can be NH or O; R² can be:

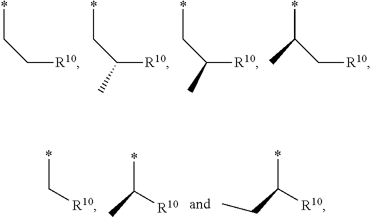

optionally substituted with one, two, three or four substituents independently selected from halo, OH, CN, oxo, —OC₁-C₄ alkyl, —OC₃-C₅ cycloalkyl, —Z—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN, wherein * indicates the connection point to Y in formula (I); and Z can be a bond,

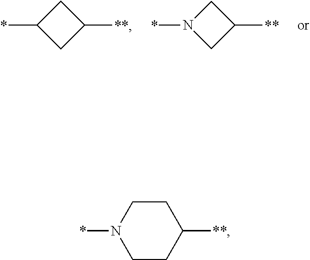

wherein * indicates the connection point to Z₁ and ** indicates the connection point to A in formula (I).

In the compounds of formula (I), A can be:

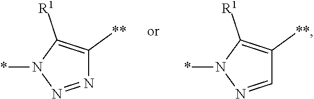

wherein * indicates the connection point to Z or Z′ and ** indicates the other connection point from A in formula (I); R⁹ can CN, F, Cl or CF₃; Y can be NH or O; and R² can be:

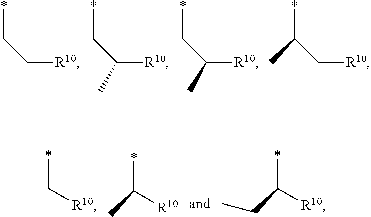

optionally substituted with one, two, three or four substituents independently selected from F, OH, CN, oxo, —OCH₃, —OC₃ cycloalkyl and R¹⁰, wherein * indicates the connection point to Y in formula (I); and Z can be a bond,

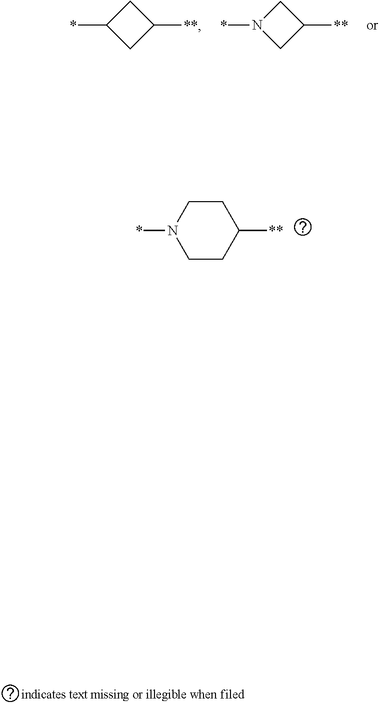

wherein * indicates the connection point to Z₁ and ** indicates the connection point to A in formula (I).

In the compounds of formula (I), A can be pyrazole, 1,2,3 triazole, or 1,2,4 triazole, substituted with R¹ and R^{1A}, R⁶ can be CN, F, Cl, CH₂, CF₃ or cyclopropyl; Y can be NH or O; R² can be C₁-C₃ alkyl optionally substituted with one, two, three or four substituents independently selected from halo, OH, CN, oxo, —OC₁-C₄ alkyl, —OC₃-C₅ cycloalkyl, —Z₂—R¹¹ and R¹⁰, wherein C₁-C₄ alkyl and C₃-C₅ cycloalkyl are optionally substituted with one or more substituents independently selected from halo, OH, OCH₃, methylamine, N,N-dimethylamine and CN; Z can be a bond,

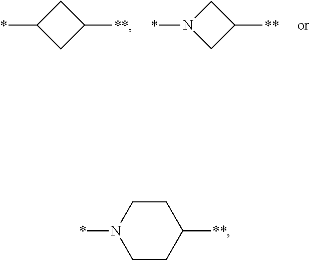

wherein * indicates the connection point to Z₁ and ** indicates the connection point to A in formula (I); Y₁ can be a bond or CHR⁷, wherein R⁷ is hydrogen, F, OH or CH₂; and Y₂ can a bond or CHR³, wherein R³ is hydrogen, F, OH or CH₃. In the compounds of formula (I), A can be pyrazole, 1,2,3 triazole, or 1,2,4 triazole, substituted with R¹ and RIA; R⁹ can be CN, F, Cl, CH₂, CF₃ or cyclopropyl; Y can be NH or O; R² can be C₁-C₄ alkyl optionally substituted with one or more substituents independently selected from F, OH, CN, oxo, —OCH₃, —OC₃ cycloalkyl and R¹⁰; Z can be a bond,

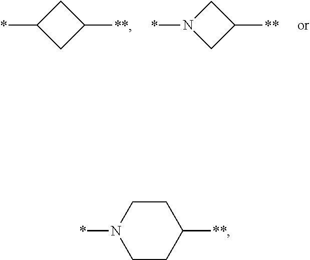

wherein * indicates the connection point to Z₁ and ** indicates the connection point to A in formula (I); Y₁ can be a bond or CHR⁷, wherein R⁷ is hydrogen, F, OH or CH₂, and Y₂ can a bond or CHR³, wherein R³ is hydrogen, F, OH or CH₃.

In the compounds of formula (I), A can be:

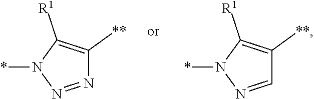

wherein * indicates the connection point to Z or Z′ and ** indicates the other connection point from A in formula (I); R⁶ can CN, F, Cl or CF₃; Y can be NH or O; and R² can be:

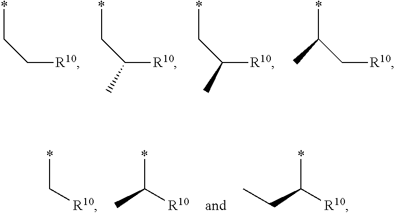

optionally substituted with one, two, three or four substituents independently selected from F, OH, CN, oxo, —OCH₃, —OC₃ cycloalkyl and R¹⁰, wherein * indicates the connection point to Y in formula (I); Z can be a bond,

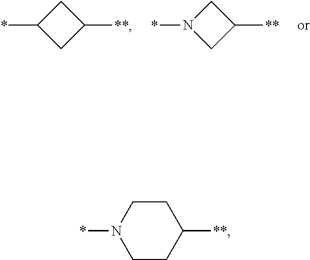

wherein * indicates the connection point to Z₁ and ** indicates the connection point to A in formula (I); Y₁ can be a bond or CHR⁷, wherein R⁷ is hydrogen, F, OH or CH₂; and Y₂ can a bond or CHR³, wherein R³ is hydrogen, F, OH or CH₃.

In the compounds of formula (II), (IIA) or (III), A can be pyrazole, 1,2,3 triazole, or 1,2,4 triazole, substituted with R¹; Y can be O; Re can be CN or Cl; R² can be C₁-C₃ alkyl optionally substituted with one, two or three substituents independently selected from OH, methoxy, halomethyl and R¹⁰, Y₁ can be a bond, CH₂, or CH₂—CH₂; and Ya can be a bond, CH₂, CF₂ or CH₂—CH₂.

In the compounds of formula (II), (IIA) or (III), A can be:

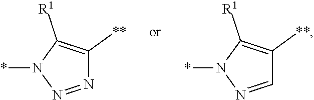

wherein * indicates the connection point to Z or Z′ and ** indicates the other connection point from A in formula (II), (IIA) or (III); Y can be O; R⁶ can be CN or Cl; R² can be C₁-C₃ alkyl optionally substituted with one, two or three substituents independently selected from OH, methoxy, halomethyl and R¹⁰, Y₁ can be a bond, CH₂, or CH₂—CH₂; and Y₂ can be a bond, CH₂, CF₂ or CH₂—CH₂

In the compounds of formula (II) or (IIA), A can be pyrazole, 1,2,3 triazole, or 1,2,4 triazole, substituted with R³; Y can be O; R⁶ can be CN or Cl; R² can be C₁-C₃ alkyl optionally substituted with one, two or three substituents independently selected from OH, methoxy, halomethyl and R¹⁰; Y₁ can be a bond, CH₂, or CH₂—CH₂; Y₂ can be a bond, CH₂, CF₂ or CH₂—CH₂; Z can be CHR^{9A}, Z₁ can be CH₂; and R⁹ can be fused with R^{9A} to form CH₂.

In the compounds of formula (II) or (IIA), A can be:

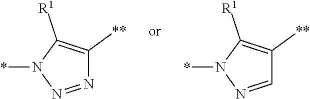

or wherein * indicates the connection point to Z and ** indicates the other connection point from A in formula (II) or (IIA); Y can be O; Re can be CN or Cl; R² can be C₁-C₃ alkyl optionally substituted with one, two or three substituents independently selected from OH, methoxy, halomethyl and R¹⁰; Y₁ can be a bond, CH₂, or CH₂—CH₂; and Y₂ can be a bond, CH₂, CF₂ or CH₂—CH₂; Z can be CHR^{9A}; Z₁ can be CH₂; and R^{9A} can be fused with R^{9A} to form CH₂.

In the compounds of formula (III), A can be pyrazole, 1,2,3 triazole, or 1,2,4 triazole, substituted with R¹; Y can be O; R⁵ can be CN or Cl; R² can be C₁-C₃ alkyl optionally substituted with one, two or three substituents independently selected from OH, methoxy, halomethyl and R¹⁰; Y₁ can be a bond, CH₂, or CH₂—CH₂, Y₂ can be a bond, CH₂, CF₂ or CH₂—CH₂; and Z′ can be:

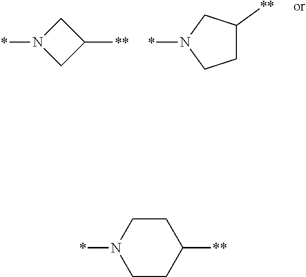

or wherein ** indicates the connection point to A and * indicates the other connection point from Z′.

In the compounds of formula (III), A can be:

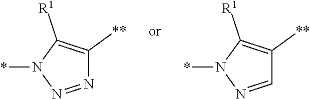

or wherein * indicates the connection point to Z′ and ** indicates the other connection point from A in formula (I); Y can be O; R⁶ can be CN or Cl; R² can be C₁-C₃ alkyl optionally substituted with one, two or three substituents independently selected from OH, methoxy, halomethyl and R¹⁰; Y₁ can be a bond, CH₂, or CH₂—CH₂; and Y₂ can be a bond, CH₂, CF₂ of CH₂—CH₂; and Z′ can be:

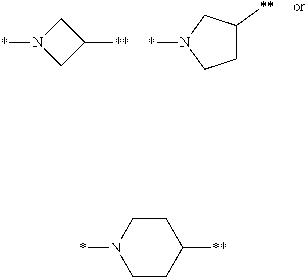

or wherein ** indicates the connection point to A and * indicates the other connection point from Z′.

In one embodiment, the compounds of Formula (I) are selected from the group consisting of:

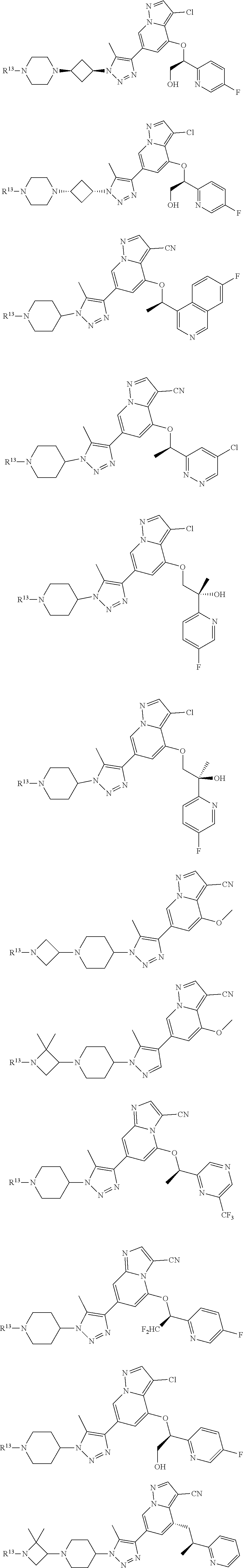

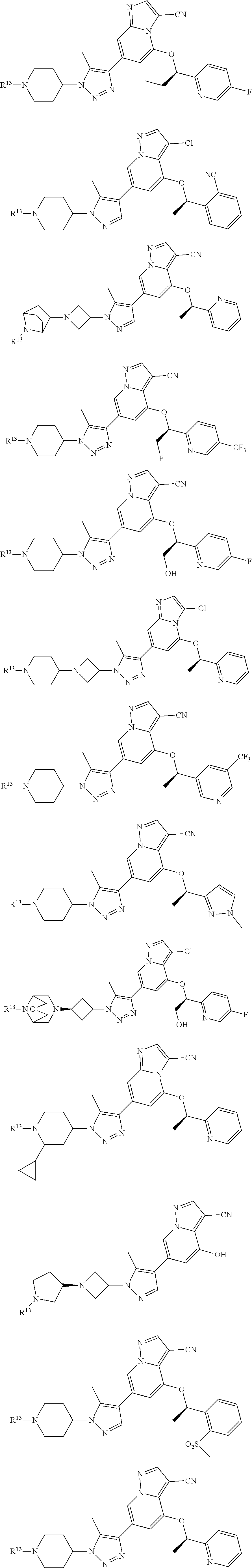

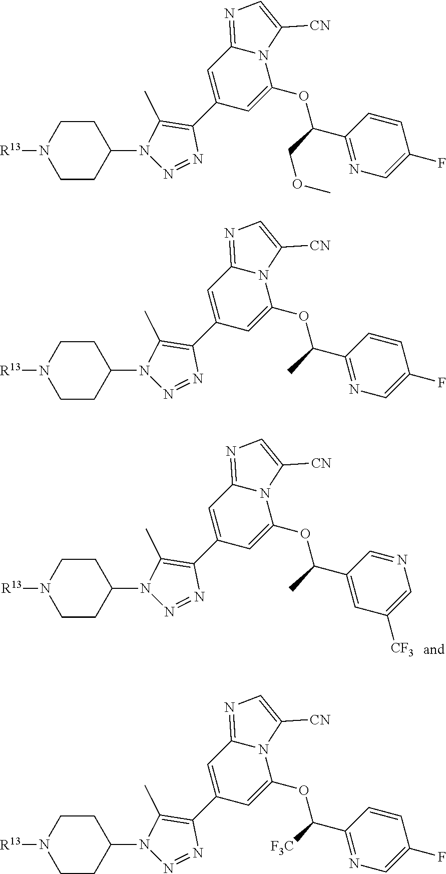

or a stereoisomer thereof, or a pharmaceutically acceptable salt thereof.

For example, for the compound of formula:

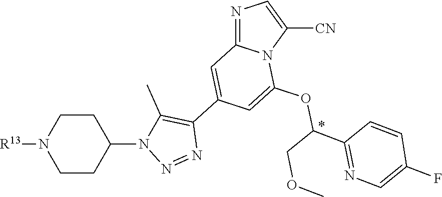

where the bond at the * position is as represented,

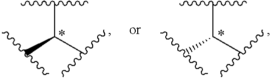

forms the compounds:

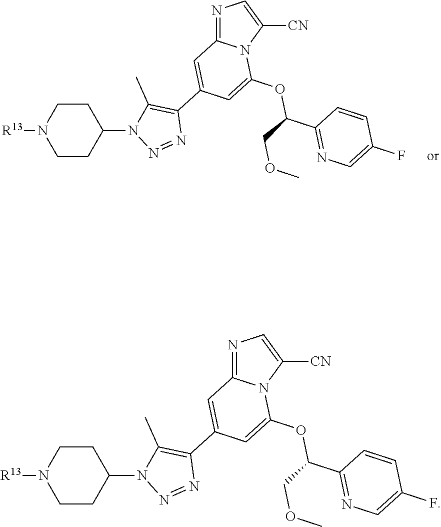

In some aspects, the disclosure is directed to the following compounds, or pharmaceutically acceptable salts thereof, shown in Table 1 below.

TABLE 1
Compounds of Formula I

| Name | Structure |
|---|---|
| 4-[2-(dimethylamino)ethoxy]-6-[5- methyl-1-(1-prop-2-enoyl-4- piperidyl)triazol-4-yl]pyrazolo [1,5- a]pyridine-3-carbonitrile | 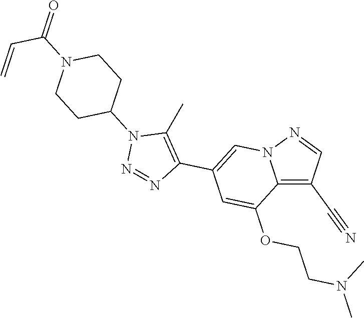 [SMILES: C=CC(=O)N1CCC(n2nnc(-c3cc(OCCN(C)C)c4c(C#N)cnn4c3)c2C)CC1] |
| 4-[2-(dimethylamino)ethoxy]-6-[5- methyl-1-(1-propanoyl-4- piperidyl)triazol-4-yl]pyrazolo[1,5- a]pyridine-3-carbonitrile | 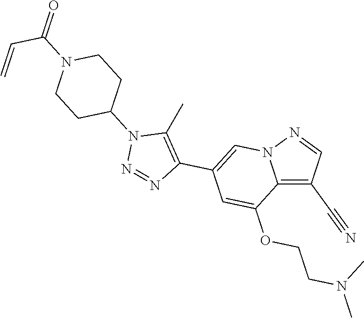 [SMILES: C=CC(=O)N1CCC(n2nnc(-c3cc(OCCN(C)C)c4c(C#N)cnn4c3)c2C)CC1] |
| 4-(ethylamino)-6-[1-[1-(3-fluoro-1- prop-2-enoyl-azetidine-3-carbonyl)- 4-piperidyl]-5-methyl-triazol-4- yl]pyrazolo[1,5-a]pyridine-3- carbonitrile | 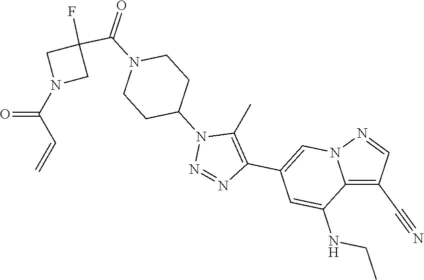 [SMILES: C=CC(=O)N1CC(F)(C(=O)N2CCC(n3nnc(-c4cc(NCC)c5c(C#N)cnn5c4)c3C)CC2)C1] |
| 4-(ethylamino)-6-[1-[1-(3-fluoro-1- propanoyl-azetidine-3-carbonyl)-4- piperidyl]-5-methyl-triazol-4- yl]pyrazolo[1,5-a]pyridine-3- carbonitrile | 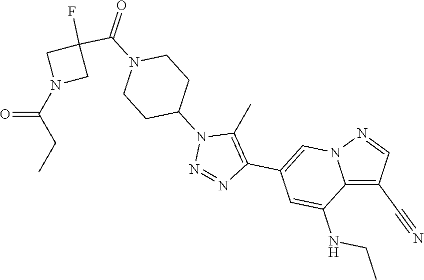 [SMILES: CCNc1cc(-c2nnn(C3CCN(C(=O)C4(F)CN(C(=O)CC)C4)CC3)c2C)cn2ncc(C#N)c12] |
| 4-(ethylamino)-6-[1-[1-(3-fluoro-1- prop-2-ynoyl-azetidine-3-carbonyl)- 4-piperidyl]-5-methyl-triazol-4- yl]pyrazolo[1,5-a]pyridine-3- carbonitrile | 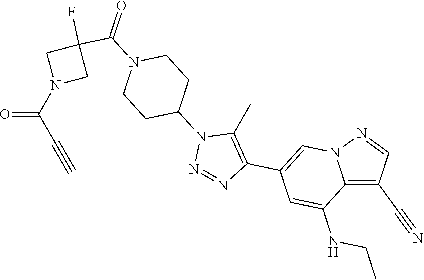 [SMILES: C#CC(=O)N1CC(F)(C(=O)N2CCC(n3nnc(-c4cc(NCC)c5c(C#N)cnn5c4)c3C)CC2)C1] |
| 6-[1-[1-(1-but-2-ynoyl-3-fluoro- azetidine-3-carbonyl)-4-piperidyl]- 5-methyl-triazol-4-yl]-4-(ethyl- amino)pyrazolo[1,5-a]pyridine-3- carbonitrile | 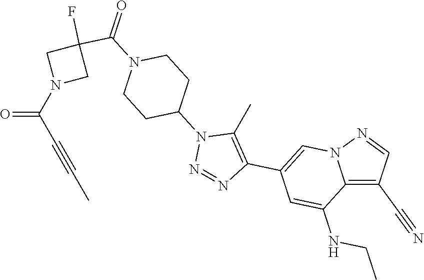 [SMILES: CC#CC(=O)N1CC(F)(C(=O)N2CCC(n3nnc(-c4cc(NCC)c5c(C#N)cnn5c4)c3C)CC2)C1] |
| 6-[1-[1-(3-fluoro-1-prop-2-enoyl- azetidine-3-carbonyl)-4-piperidyl]- 5-methyl-triazol-4-yl]-4-(methyl- amino)pyrazolo[1,5-a]pyridine-3- carbonitrile | 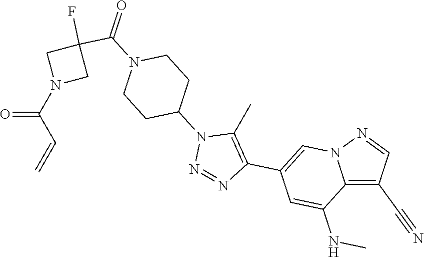 [SMILES: C=CC(=O)N1CC(F)(C(=O)N2CCC(n3nnc(-c4cc(NC)c5c(C#N)cnn5c4)c3C)CC2)C1] |
| 6-[1-[1-(3-fluoro-1-prop-2-enoyl- azetidine-3-carbonyl)-4-piperidyl]- 5-methyl-triazol-4-yl]-4- (isopropylamino)pyrazolo[1,5- a]pyridine-3-carbonitrile | 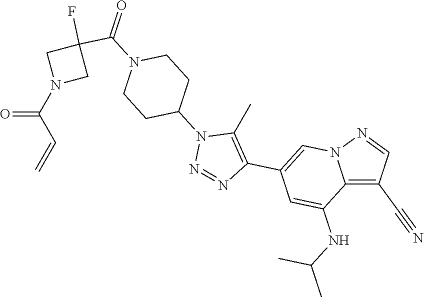 [SMILES: C=CC(=O)N1CC(F)(C(=O)N2CCC(n3nnc(-c4cc(NC(C)C)c5c(C#N)cnn5c4)c3C)CC2)C1] |
| 6-[1-[1-(3-fluoro-1-prop-2-enoyl- azetidine-3-carbonyl)-4-piperidyl]- 5-methyl-triazol-4-yl]-4-(propyl- amino)pyrazolo[1,5-a]pyridine-3- carbonitrile | 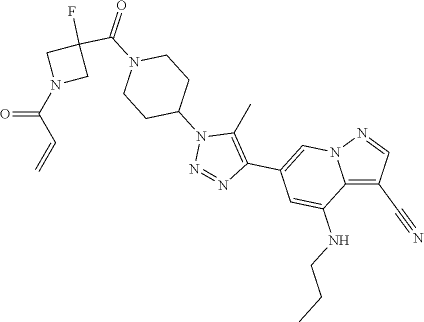 [SMILES: C=CC(=O)N1CC(F)(C(=O)N2CCC(n3nnc(-c4cc(NCCC)c5c(C#N)cnn5c4)c3C)CC2)C1] |
| 6-[1-[1-(3-fluoro-1-prop-2-enoyl- azetidine-3-carbonyl)-4-piperidyl]- 5-methyl-triazol-4-yl]-4-(2- hydroxyethylamino)-pyrazolo[1,5- a]pyridine-3-carbonitrile | 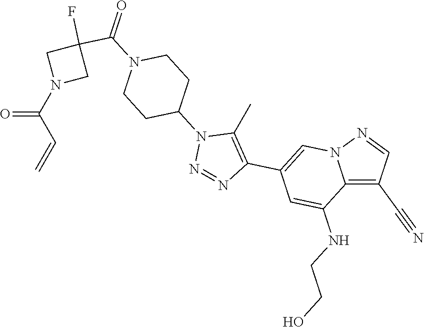 [SMILES: C=CC(=O)N1CC(F)(C(=O)N2CCC(n3nnc(-c4cc(NCCO)c5c(C#N)cnn5c4)c3C)CC2)C1] |
| 6-[1-[1-(3-fluoro-1-prop-2-enoyl- azetidine-3-carbonyl)-4-piperidyl]- 5-methyl-triazol-4-yl]-4-(2- methoxyethylamino)-pyrazolo[1,5- a]pyridine-3-carbonitrile | 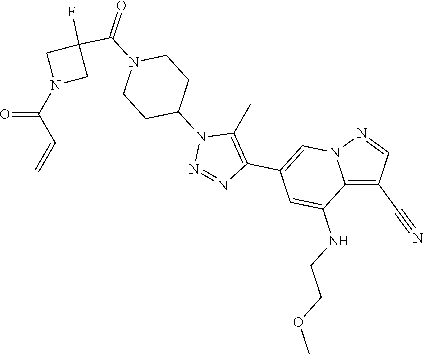 [SMILES: C=CC(=O)N1CC(F)(C(=O)N2CCC(n3nnc(-c4cc(NCCOC)c5c(C#N)cnn5c4)c3C)CC2)C1] |
| 6-[1-[1-(3-fluoro-1-prop-2-enoyl- azetidine-3-carbonyl)-4-piperidyl]- 5-methyl-triazol-4-yl]-4-(2- methoxyethylamino)pyrazolo[1,5- a]pyridine-3-carbonitrile | 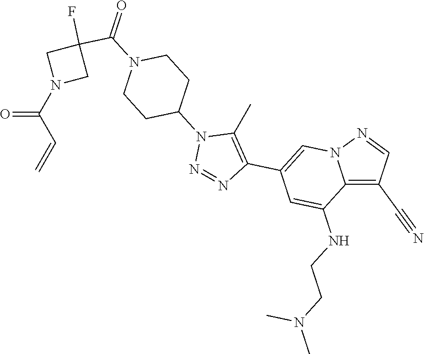 [SMILES: C=CC(=O)N1CC(F)(C(=O)N2CCC(n3nnc(-c4cc(NCCN(C)C)c5c(C#N)cnn5c4)c3C)CC2)C1] |
| 4-(allylamino)-6-[1-[1-(3-fluoro-1- prop-2-enoyl-azetidine-3-carbonyl)- 4-piperidyl]-5-methyl-triazol-4- yl]pyrazolo[1,5-a]pyridine-3- carbonitrile | 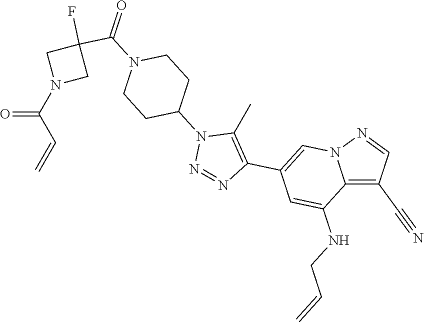 [SMILES: C=CCNc1cc(-c2nnn(C3CCN(C(=O)C4(F)CN(C(=O)C=C)C4)CC3)c2C)cn2ncc(C#N)c12] |
| 4-(cyclopropylmethylamino)-6-[1- [1-(3-fluoro-1-prop-2-enoyl- azetidine-3-carbonyl)-4-piperidyl]- 5-methyl-triazol-4-yl]pyrazolo [1,5- a]pyridine-3-carbonitrile | 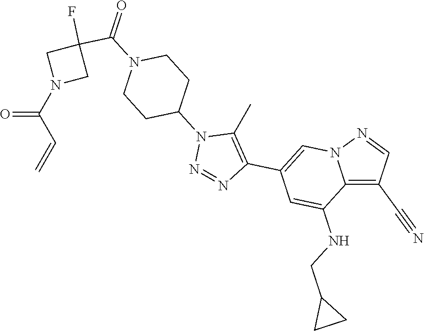 [SMILES: C=CC(=O)N1CC(F)(C(=O)N2CCC(n3nnc(-c4cc(NCC5CC5)c5c(C#N)cnn5c4)c3C)CC2)C1] |
| 4-(cyclopropylmethylamino)-6-[1- [1-(3-fluoro-1-prop-2-enoyl- azetidine-3-carbonyl)-4-piperidyl]- 3,5-dimethyl-pyrazol-4- yl]pyrazolo[1,5-a]pyridine-3- carbonitrile | 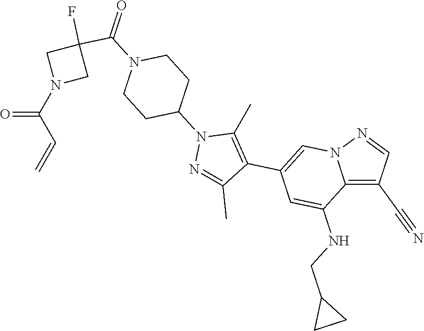 [SMILES: C=CC(=O)N1CC(F)(C(=O)N2CCC(n3nc(C)c(-c4cc(NCC5CC5)c5c(C#N)cnn5c4)c3C)CC2)C1] |
| 4-(ethylamino)-6-[1-[1-(3-fluoro-1- prop-2-enoyl-azetidine-3-carbonyl)- 4-piperidyl]-3,5-dimethyl-pyrazol-4- yl]pyrazolo [1,5-a]pyridine-3- carbonitrile | 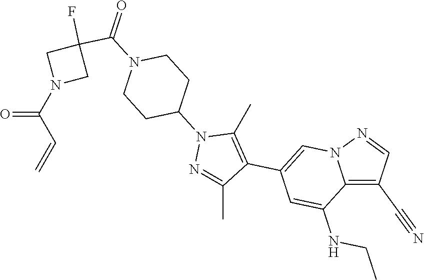 [SMILES: C=CC(=O)N1CC(F)(C(=O)N2CCC(n3nc(C)c(-c4cc(NCC)c5c(C#N)cnn5c4)c3C)CC2)C1] |
| 6-[1-[1-(3-fluoro-1-prop-2-enoyl- azetidine-3-carbonyl)-4-piperidyl]- 3,5-dimethyl-pyrazol-4-yl]-4-(2- hydroxyethylamino)-pyrazolo[1,5- a]pyridine-3-carbonitrile | 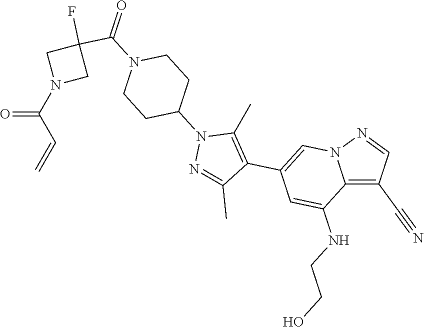 [SMILES: C=CC(=O)N1CC(F)(C(=O)N2CCC(n3nc(C)c(-c4cc(NCCO)c5c(C#N)cnn5c4)c3C)CC2)C1] |
| 6-[1-[1-(3-fluoro-1-prop-2-enoyl- azetidine-3-carbonyl)-4-piperidyl]- 3,5-dimethyl-pyrazol-4-yl]-4-(2- methoxyethylamino)-pyrazolo[1,5- a]pyridine-3-carbonitrile | 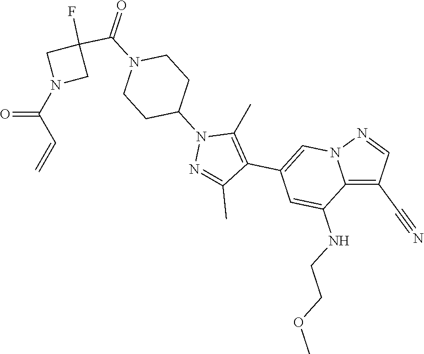 [SMILES: C=CC(=O)N1CC(F)(C(=O)N2CCC(n3nc(C)c(-c4cc(NCCOC)c5c(C#N)cnn5c4)c3C)CC2)C1] |

In one embodiment, the compounds of Formula (I) are selected from the group consisting of:
4-[1-(5-Fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile;
4-[1-(5-Fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile;
(R)-6-(1-(7-acryloyl-7-azaspiro[3.5]nonan-2-yl)-5-methyl-1H-1,2,3-triazol-4-yl)-4-(1-(pyridin-2-yl)ethoxy)pyrazolo[1,5-a]pyridine-3-carbonitrile;
(R)-1-(2-(4-(3-chloro-4-(1-(pyridin-2-yl)ethoxy)-pyrazolo[1,5-a]pyridin-6-yl)-5-methyl-1H-1,2,3-triazol-1-yl)-7-azaspiro[3.5]nonan-7-yl)prop-2-en-1-one;
(R)-1-(2-(4-(3-chloro-4-(2-hydroxy-1-(pyridin-2-yl)ethoxy)pyrazolo[1,5-a]pyridin-6-yl)-5-methyl-1H-pyrazol-1-yl)-7-azaspiro[3.5]nonan-7-yl)prop-2-en-1-one;
(S)-1-(2-(4-(3-chloro-4-(2-hydroxy-1-(pyridin-2-yl)ethoxy)pyrazolo[1,5-a]pyridin-6-yl)-5-methyl-1H-pyrazol-1-yl)-7-azaspiro[3.5]nonan-7-yl)prop-2-en-1-one;
(R)-6-(1-(7-acryloyl-7-azaspiro[3.5]nonan-2-yl)-5-methyl-1H-pyrazol-4-yl)-4-(2-hydroxy-1-(pyridin-2-yl)ethoxy)pyrazolo[1,5-a]pyridine-3-carbonitrile;
(S)-6-(1-(7-acryloyl-7-azaspiro[3.5]nonan-2-yl)-5-methyl-1H-pyrazol-4-yl)-4-(2-hydroxy-1-(pyridin-2-yl)ethoxy)pyrazolo[1,5-a]pyridine-3-carbonitrile;
(S)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile;
(R)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile;
6-(1-(7-acryloyl-7-azaspiro[3.5]nonan-2-yl)-5-methyl-1H-pyrazol-4-yl)-4-(2-hydroxy-1-(1-methyl-1H-pyrazol-5-yl)ethoxy)pyrazolo[1,5-a]pyridine-3-carbonitrile;
(R)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile;
(S)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile;
6-(1-(7-acryloyl-7-azaspiro[3.5]nonan-2-yl)-5-methyl-1H-pyrazol-4-yl)-4-(2-hydroxy-1-(1-methyl-1H-pyrazol-3-yl)ethoxy)pyrazolo[1,5-a]pyridine-3-carbonitrile;
6-(1-(7-acryloyl-7-azaspiro[3.5]nonan-2-yl)-5-methyl-1H-pyrazol-4-yl)-4-(2-hydroxy-1-(isoquinolin-4-yl)ethoxy)pyrazolo[1,5-a]pyridine-3-carbonitrile;
4-(2-hydroxy-1-(pyridin-2-yl)ethoxy)-6-(5-methyl-1-(1-(1-propionylazetidine-3-carbonyl)piperidin-4-yl)-1H-1,2,3-triazol-4-yl)pyrazolo[1,5-a]pyridine-3-carbonitrile;
(R)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(S)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(R)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(S)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(R)-6-(1-(1-(1-acryloyl-3-methoxyazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(S)-6-(1-(1-(1-acryloyl-3-methoxyazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(E)-6-(1-(1-(1-(4-(dimethylamino)but-2-enoyl)-3-methoxyazetidine-3-carbonyl) piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
4-(2-hydroxy-1-(pyridin-2-yl)ethoxy)-6-(1-(1-(3-methoxy-1-propioloylazetidine-3-carbonyl)piperidin-4-yl)-5-methyl-1H-1,2,3-triazol-4-yl)pyrazolo[1,5-a]pyridine-3-carbonitrile;
(R)-6-(1-(7-acryloyl-7-azaspiro[3.5]nonan-2-yl)-1H-pyrazol-4-yl)-4-(1-(5-fluoropyridin-2-yl)-2-hydroxyethoxy)pyrazolo[1,5-a]pyridine-3-carbonitrile;
(R)-4-(1-(5-fluoropyridin-2-yl)-2-hydroxyethoxy)-6-(1-(7-propionyl-7-azaspiro[3.5]nonan-2-yl)-1H-pyrazol-4-yl)pyrazolo[1,5-a]pyridine-3-carbonitrile;
(S)-6-(1-(7-acryloyl-7-azaspiro[3.5]nonan-2-yl)-1H-pyrazol-4-yl)-4-(1-(5-fluoropyridin-2-yl)-2-hydroxyethoxy)pyrazolo[1,5-a]pyridine-3-carbonitrile;
(S)-4-(1-(5-fluoropyridin-2-yl)-2-hydroxyethoxy)-6-(1-(7-propionyl-7-azaspiro[3.5]nonan-2-yl)-1H-pyrazol-4-yl)pyrazolo[1,5-a]pyridine-3-carbonitrile;
6-(1-(1-(1-acryloyl-3-fluoroazetidine-3-carbonyl)piperidin-4-yl)-5-methyl-1H-1,2,3-triazol-4-yl)-4-(2-(dimethylamino)-ethoxy)pyrazolo[1,5-a]pyridine-3-carbonitrile;
4-(2-(dimethylamino) ethoxy)-6-(1-(1-(3-fluoro-1-propionylazetidine-3-carbonyl) piperidin-4-yl)-5-methyl-1H-1,2,3-triazol-4-yl)pyrazolo[1,5-a]pyridine-3-carbonitrile;
or a stereoisomer thereof, or a pharmaceutically acceptable salt thereof.

In one embodiment, the compounds of Formula (I) are selected from the group consisting of:
4-[2-(dimethylamino) ethoxy]-6-[5-methyl-1-(1-prop-2-enoyl-4-piperidyl)triazol-4-yl]pyrazolo[1,5-a]pyridine-3-carbonitrile;
4-[2-(dimethylamino) ethoxy]-6-[5-methyl-1-(1-propanoyl-4-piperidyl)triazol-4-yl]pyrazolo[1,5-a]pyridine-3-carbonitrile;
4-(ethylamino)-6-[1-[1-(3-fluoro-1-prop-2-enoyl-azetidine-3-carbonyl)-4-piperidyl]-5-methyl-triazol-4-yl]pyrazolo[1,5-a]pyridine-3-carbonitrile;
4-(ethylamino)-6-[1-[1-(3-fluoro-1-propanoyl-azetidine-3-carbonyl)-4-piperidyl]-5-methyl-triazol-4-yl]pyrazolo[1,5-a]pyridine-3-carbonitrile;
4-(ethylamino)-6-[1-[1-(3-fluoro-1-prop-2-ynoyl-azetidine-3-carbonyl)-4-piperidyl]-5-methyl-triazol-4-yl]pyrazolo[1,5-a]pyridine-3-carbonitrile;
6-[1-[1-(1-but-2-ynoyl-3-fluoro-azetidine-3-carbonyl)-4-piperidyl]-5-methyl-triazol-4-yl]-4-(ethyl-amino)pyrazolo[1,5-a]pyridine-3-carbonitrile;
6-[1-[1-(3-fluoro-1-prop-2-enoyl-azetidine-3-carbonyl)-4-piperidyl]-5-methyl-triazol-4-yl]-4-(methyl-amino)pyrazolo[1,5-a]pyridine-3-carbonitrile;
6-[1-[1-(3-fluoro-1-prop-2-enoyl-azetidine-3-carbonyl)-4-piperidyl]-5-methyl-triazol-4-yl]-4-(isopropylamino)pyrazolo[1,5-a]pyridine-3-carbonitrile;
6-[1-[1-(3-fluoro-1-prop-2-enoyl-azetidine-3-carbonyl)-4-piperidyl]-5-methyl-triazol-4-yl]-4-(propyl-amino)pyrazolo[1,5-a]pyridine-3-carbonitrile;
6-[1-[1-(3-fluoro-1-prop-2-enoyl-azetidine-3-carbonyl)-4-piperidyl]-5-methyl-triazol-4-yl]-4-(2-hydroxyethylamino)-pyrazolo[1,5-a]pyridine-3-carbonitrile;
6-[1-[1-(3-fluoro-1-prop-2-enoyl-azetidine-3-carbonyl)-4-piperidyl]-5-methyl-triazol-4-yl]-4-(2-methoxyethylamino)-pyrazolo[1,5-a]pyridine-3-carbonitrile;
6-[1-[1-(3-fluoro-1-prop-2-enoyl-azetidine-3-carbonyl)-4-piperidyl]-5-methyl-triazol-4-yl]-4-(2-methoxyethylamino)pyrazolo[1,5-a]pyridine-3-carbonitrile;
4-(allylamino)-6-[1-[1-(3-fluoro-1-prop-2-enoyl-azetidine-3-carbonyl)-4-piperidyl]-5-methyl-triazol-4-yl]pyrazolo[1,5-a]pyridine-3-carbonitrile;
4-(cyclopropylmethylamino)-6-[1-[1-(3-fluoro-1-prop-2-enoyl-azetidine-3-carbonyl)-4-piperidyl]-5-methyl-triazol-4-yl]pyrazolo[1,5-a]pyridine-3-carbonitrile;
4-(cyclopropylmethylamino)-6-[1-[1-(3-fluoro-1-prop-2-enoyl-azetidine-3-carbonyl)-4-piperidyl]-3,5-dimethyl-pyrazol-4-yl]pyrazolo[1,5-a]pyridine-3-carbonitrile;
4-(ethylamino)-6-[1-[1-(3-fluoro-1-prop-2-enoyl-azetidine-3-carbonyl)-4-piperidyl]-3,5-dimethyl-pyrazol-4-yl]pyrazolo[1,5-a]pyridine-3-carbonitrile;
6-[1-[1-(3-fluoro-1-prop-2-enoyl-azetidine-3-carbonyl)-4-piperidyl]-3,5-dimethyl-pyrazol-4-yl]-4-(2-hydroxyethylamino)-pyrazolo[1,5-a]pyridine-3-carbonitrile;
6-[1-[1-(3-fluoro-1-prop-2-enoyl-azetidine-3-carbonyl)-4-piperidyl]-3,5-dimethyl-pyrazol-4-yl]-4-(2-methoxyethylamino)-pyrazolo[1,5-a]pyridine-3-carbonitrile;
or a pharmaceutically acceptable salt thereof.

The compounds of formula (I), (II), (IIA) or (III), provided herein, or a pharmaceutically acceptable salt thereof, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl refers to both C₂H₅ or C₂H₅ where from 1 to 5 hydrogens are replaced by deuterium, such as in C₂D_{X}H_{5-X}.

The compounds of formula (I), (II), (IIA) or (III) provided herein may form pharmaceutically acceptable salts. The Examples provided herein may form pharmaceutically acceptable salts. Such pharmaceutically acceptable salts are intended to be included. Pharmaceutically acceptable salts and common methodology for preparing them are well known in the art (see, e.g., P. Stahl, et al. Handbook of Pharmaceutical Salts: Properties, Selection and Use, 2^{nd} Revised Edition (Wiley-VCH, 2011); S. M. Berge, et al, “Pharmaceutical Salts,” Journal of Pharmaceutical Sciences, Vol. 66, No. 1, January 1977).

The compounds of formula (I), (II), (IIA) or (III) provided herein, or a pharmaceutically acceptable salt thereof, can be mixed with one or more pharmaceutically acceptable carriers, diluents, or excipients. More particularly, the compounds of formula (I), (II), (IIA) or (III) provided herein, or a pharmaceutically acceptable salt thereof, can be formulated as pharmaceutical compositions. Such pharmaceutical compositions and processes for preparing the same are well known in the art (see, e.g., Remington: The Science and Practice of Pharmacy (A. Gennaro, et al., eds., 21st ed., Mack Publishing Co., 2005)).

The compounds of formula (I), (II), (IIA) or (III) provided herein, or a pharmaceutically acceptable salt thereof, and their pharmaceutical compositions can be administered by a variety of routes. Such routes of administration include oral and intravenous.

The compounds of formula (I), (II), (IIA) or (III) provided herein, or a pharmaceutically acceptable salt thereof, can be combined with one or more other therapeutic agents.

The compounds of formula (I), (II), (IIA) or (III) provided herein, or a pharmaceutically acceptable salt thereof, can be a component in a pharmaceutical composition for the treatment of systemic sclerosis, fibrosis, pulmonary fibrosis, achondroplasia, thanatophoric dysplasia, severe achondroplasia with developmental delay and acanthosis nigricans (SADDAN), muenke syndrome or cancer with one or more pharmaceutically acceptable carriers, diluents, or excipients, and optionally with one or more additional therapeutic agents.

The compounds of formula (I), (II), (IIA) or (III) provided herein, or a pharmaceutically acceptable salt thereof, can be a component in a pharmaceutical composition for the treatment of cancer with one or more pharmaceutically acceptable carriers, diluents, or excipients, and optionally with one or more additional therapeutic agents.

The compounds of formula (I), (II), (IIA) or (III) provided herein, or a pharmaceutically acceptable salt thereof, can be combined with one or more other therapeutic agents for simultaneous, separate or sequential administration.

The compounds of formula (I), (II), (IIA) or (III) provided herein, or a pharmaceutically acceptable salt thereof, and their pharmaceutical compositions can be used in the methods described herein.

The compounds of formula (I), (II), (IIA) or (III) provided herein, or a pharmaceutically acceptable salt thereof, are generally effective over a wide dosage range. For example, dosages per day normally fall within the range of about 0.5 to about 100 mg/kg of body weight. In some instances, dosage levels below the lower limit of the aforesaid range may be more than adequate, while in other cases still larger doses may be employed without causing any harmful side effect, and therefore the above dosage range is not intended to limit the scope of the invention in any way. It will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms.

Certain compounds of formula (I), (II), (IIA) or (III), or a pharmaceutically acceptable salt thereof, selectively target FGFR3. For example, certain compounds of formula (I), (II), (IIA) or (III), or a pharmaceutically acceptable salt thereof, selectively target FGFR3 over another FGFR. For example, certain compounds of formula (I), (II), (IIA) or (III), or a pharmaceutically acceptable salt thereof, selectively target FGFR3 over FGFR1. For example, certain compounds of formula (I), (II), (IIA) or (III), or a pharmaceutically acceptable salt thereof, are at least about 3 fold (e.g., at least about 4-, 5-, 6-, 7-, 8-, 9-, 10-, 15-, 20-, 30-, 40-, 50-fold, or more) more selective for FGFR3 than for FGFR1.

As used herein, the term “selectivity” of a compound refers to the compound having more potent activity at the first target than the second target. A fold selectivity can be calculated by any method known in the art. For example, a fold selectivity can be calculated by dividing the IC₅₀ value of a compound for the second target (e.g., FGFR1) by the IC₅₀ value of the same compound for the first target (e.g., FGFR3). An IC₅₀ value can be determined by any method known in the art. For example, an IC₅₀ value can be determined as described in the assays below.

As used herein, the term “cancer” refers to or describes the physiological condition in patients that is typically characterized by unregulated cell proliferation. Included in this definition are benign and malignant cancers.

As used herein, the term “FGFR3-associated cancer” refers to cancers associated with or having a dysregulation of the FGFR3 gene, the FGFR3 kinase protein, or expression or activity, or level of any of the same. Non-limiting examples of FGFR3-associated cancer are described herein. As used herein an “FGFR3-associated cancer” includes but is not limited to breast cancer (e.g. invasive ductal cancer, invasive lobular cancer), lung cancer (e.g. non-small-cell lung cancer, lung adenocarcinoma, squamous cell lung cancer and small-cell lung cancer), urothelial cancer, bladder cancer (e.g. urothelial bladder cancer, non-muscle invasive bladder cancer, muscle invasive bladder cancer), upper tract cancer (e.g. urothelial upper tract cancer), urethral cancer, gastric cancer, pancreatic cancer, prostate cancer, colorectal cancer, multiple myeloma, liver cancer, melanoma (e.g. cutaneous melanoma), head and neck cancer (e.g. oral cancer), thyroid cancer, renal cancer (e.g. renal pelvis cancer), glioblastoma, endometrial cancer, cervical cancer, ovarian cancer, and testicular cancer.

As used herein, the term “treating” (or “treatment”) refers to restraining, slowing, stopping, or reversing the progression or severity of an existing symptom, condition or disorder.

As used herein, the term “patient” refers to a mammal, particularly a human.

Provided herein, are compounds of formula (I), (II), (IIA) or (III), or a pharmaceutically acceptable salt thereof, for use in therapy.

Provided herein, are compounds of formula (I), (II), (IIA) or (III), or a pharmaceutically acceptable salt thereof, for use in the treatment of systemic sclerosis, fibrosis, pulmonary fibrosis, achondroplasia, thanatophoric dysplasia, severe achondroplasia with developmental delay and acanthosis nigricans (SADDAN), muenke syndrome or cancer.

Provided herein, are compounds of formula (I), (II), (IIA) or (III), or a pharmaceutically acceptable salt thereof, for use in the treatment of systemic sclerosis, fibrosis, pulmonary fibrosis, achondroplasia, thanatophoric dysplasia, severe achondroplasia with developmental delay and acanthosis nigricans (SADDAN), muenke syndrome or cancer.

Provided herein, are compounds of formula (I), (II), (IIA) or (III), or a pharmaceutically acceptable salt thereof, for use in the treatment of cancer.

Provided herein, are the use of compounds of formula (I), (II), (IIA) or (III), or a pharmaceutically acceptable salt thereof, in the manufacture of a medicament for treating systemic sclerosis, fibrosis, pulmonary fibrosis, achondroplasia, thanatophoric dysplasia, severe achondroplasia with developmental delay and acanthosis nigricans (S ADD AN), muenke syndrome or cancer.

Provided herein, are the use of compounds of formula (I), (II), (IIA) or (III), or a pharmaceutically acceptable salt thereof, in the manufacture of a medicament for treating cancer.

Provided herein are methods of treating systemic sclerosis, fibrosis, pulmonary fibrosis, achondroplasia, thanatophoric dysplasia, severe achondroplasia with developmental delay and acanthosis nigricans (S ADD AN), muenke syndrome or cancer, comprising administering to a patient in need of such treatment an effective amount of the compounds of formula (I), (II), (IIA) or (III), or a pharmaceutically acceptable salt thereof.

Provided herein are methods of treating cancer, comprising administering to a patient in need of such treatment an effective amount of the compounds of formula (I), (II), (IIA) or (III), or a pharmaceutically acceptable salt thereof.

Provided in the methods and uses herein, the cancer is selected from the group consisting of breast cancer (e.g. invasive ductal cancer, invasive lobular cancer), lung cancer (e.g. non-small-cell lung cancer, lung adenocarcinoma, squamous cell lung cancer and small-cell lung cancer), urothelial cancer, bladder cancer (e.g. urothelial bladder cancer, non-muscle invasive bladder cancer, muscle invasive bladder cancer), upper tract cancer (e.g. urothelial upper tract cancer), urethral cancer, gastric cancer, pancreatic cancer, prostate cancer, colorectal cancer, multiple myeloma, liver cancer, melanoma (e.g. cutaneous melanoma), head and neck cancer (e.g. oral cancer), thyroid cancer, renal cancer (e.g. renal pelvis cancer), glioblastoma, endometrial cancer, cervical cancer, ovarian cancer, and testicular cancer. Particularly, the cancer is selected from the group consisting of breast cancer (e.g. invasive ductal cancer, invasive lobular cancer), lung cancer (e.g. non-small-cell lung cancer, lung adenocarcinoma, squamous cell lung cancer and small-cell lung cancer), urothelial cancer, bladder cancer (e.g. urothelial bladder cancer, non-muscle invasive bladder cancer, muscle invasive bladder cancer), upper tract cancer (e.g. urothelial upper tract cancer), urethral cancer, pancreatic cancer, prostate cancer, colorectal cancer, melanoma (e.g. cutaneous melanoma), renal cancer (e.g. renal pelvis cancer), glioblastoma, endometrial cancer, and ovarian cancer. More particularly, the cancer is selected from the group consisting of breast cancer (e.g. invasive ductal cancer, invasive lobular cancer), lung cancer (e.g. non-small-cell lung cancer, lung adenocarcinoma, squamous cell lung cancer and small-cell lung cancer), urothelial cancer, bladder cancer (e.g. urothelial bladder cancer, non-muscle invasive bladder cancer, muscle invasive bladder cancer), upper tract cancer (e.g. urothelial upper tract cancer) and glioblastoma. Most particularly, the cancer is bladder cancer (e.g., urothelial bladder cancer, non-muscle invasive bladder cancer, muscle invasive bladder cancer).

The compounds provided herein can be prepared as illustrated in the preparations and examples below.

Certain abbreviations are defined as follows: “ACN” refers to acetonitrile; “AcOH” refers to acetic acid; “Ac20” refers to acetic anhydride; “aq.” refers to aqueous, “AIBN” refers to azobisisobutyronitrile; “BINAP” refers to 2,2′-bis(diphenylphosphino)-I,G-binaphthyl; “Pd(DtBPF)Cl₂” refers to [1,1-Bis(di-tert-butylphosphino) ferrocene]dichloropalladium (II); NBS “refers to N-bromosuccinimide; “n-BuOH” refers to n-butyl alcohol or n-butanol; “BOC” refers to tert-butyloxycarbonyl; “B0C2O” refers to di-tert-butyl dicarbonate; “BuLi” refers to butyl lithium; “CuSCE 5H₂O” refers to copper sulfate pentahydrate; ‘CsF” refers to cesium fluoride; “F-TEDA” refers to 1-chloromethyl-4-fluoro-1,4-diazoniabicyclo[2.2.2]octane; “Cul” refers to copper iodide; “DMP” refers to Dess-Martin periodinane; “DCE” refers to 1,2-dichloroethane; “DCM” refers to dichloromethane or methylene chloride; “DMEA” refers to dimethylethylamine; “NDM” refers to 1-dodecanethiol; “DEA” refers to diethanolamine; “DEAD” refers to diethyl azodicarboxylate; “DIAD” refers to diisopropyl azodicarboxylate; “DIEA” or “DIPEA” refers to N,N-diisopropylethylamine; “DMA” refers to N,N-dimethyl-aniline; “DMAP” refers to 4-dimetbylaminopyridine; “DMF” refers to N,N-dimethylformamide; “DPP A” refers to diphenylphosphoryl azide; “EAA” refers to ethyl acetoacetate; “EtOAc” refers to ethyl acetate; “FA” refers to formic acid “hr” refers to hour or hours; “i-PrMgCl” refers to isopropyl magnesium chloride; “IP A” refers to isopropyl amine; “T3P” refers to propyl-phosphonic anhydride; “KOAc” refers to potassium acetate; “LiBFE” refers to lithium borohydride; “LDA” refers to lithium diisopropylamide; “MsCl” refers to methanesulfonyl chloride; “MTBE” refers to methyl tert-butyl ether; “NCS” refers to N-chlorosuccinimide; “NIS” refers to N-iodosuccinimide; ‘MeMgBr” refers to methyl magnesium bromide; “NMP” refers to N-methyl-2-pyrrolidone; “—OAc” refers to acetate; -“OMs” refers to methanesulfonate, also known as mesylate; “min” or “min.” refers to minute or minutes; “N2” refers to nitrogen; “sat.” or “sat'd” refers to saturated; “soln.” refers to solution; “—OTf’ refers to trifluoromethane-sulfonate, also known as triflate; “PCy₃” refers to tricyclohexylphosphine; “Pd(AcO)₂” refers to palladium (II) acetate; “Pd(dba)₂” refers to bis(dibenzylideneacetone)-palladium(0); “Pd₂(dba)₃” refers to tris(dibenzylideneacetone) dipalladium(O); “Pd₂(dba)₃·CHCl₃” refers to tris(dibenzylideneacetone) dipalladium-chloroform adduct; “Pd(dppf)Cl₂” refers to [1,r-bis(diphenylphosphino)ferrocene]dichloropalladium (II); “Pd(dppf)Cl₂·CH₂Cl₂” refers to l,l′-bis(diphenylphosphino) ferrocene-palladium (II) dichloride dichloromethane complex; “PE” refers to petroleum ether; “K₃PO₄” refers to potassium phosphate; “RT” refers to room temperature; “PPh₃” refers to triphenylphosphine; “Pd(PPh₃)₄” refers to tetrakis(triphenyl-phosphine)palladium(0); “Ph” refers to phenyl; “NaH” refers to sodium hydride; “TBAF” refers to tetra-n-butylammonium fluoride; “TEA” refers to triethylamine; “TFA” refers to trifluoroacetic acid; “Tf₂0” refers to trifluoromethane sulfonic anhydride; “THF” refers to tetrahydrofuran; “TsCl” refers to 4-toluenesulfonyl chloride; “TMSCF₃” refers to (trifluoromethyl)trimethylsilane; “TMSOTf’ refers to trimethyl silyltrifluoro methanesulfonate; “(CF₃SO₂)₂O” refers to trifluoromethanesulfonic anhydride; “t(R)” refers to retention time; “Xantphos” refers to 4,5-bis(diphenylphosphino)-9,9-dimethylxanthene; “X-Phos” refers to 2-di cyclohexyl phosphino-2,4,6-triisopropylbiphenyl; “XPhos Pd G2” refers to chloro(2-dicyclo-hexylphosphino-2,4′,6′-triisopropyl-1,1′ biphenyl) [2-(2′-amino-1,1′-biphenyl)]palladium(II); and “XPhos Pd G₄” refers to CAS #1599466-81-5; “ZnCI₂” refers to zinc chloride.

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994) The designations “isomer 1” and “isomer 2” refer to the compounds that elute from chiral chromatography first and second, respectively, under the conditions described herein and if chiral chromatography is initiated early in the synthesis, the same designation is applied to subsequent intermediates and examples. Where more than one chiral chromatography is conducted in the preparation, a further designation of “A” and “B” is provided where “A” refers to the compounds that elute first and “B” for those that elute second. For example, “isomer 2A” refer to the first eluting compounds from the chiral chromatography of a compound previously designated “isomer 2”. Additionally, the intermediates described in the following schemes contain a number of nitrogen or oxygen protecting groups. The variable protecting group may be the same or different in each occurrence depending on the particular reaction conditions and the particular transformations to be performed. The protection and deprotection conditions are well known to the skilled artisan and are described in the literature (See for example “Greene's Protective Groups in Organic Synthesis”, Fourth Edition, by Peter G. M. Wuts and Theodora W. Greene, John Wiley and Sons, Inc. 2007).

In the schemes below, all substituents, unless otherwise indicated, are as previously defined. “PG” refers to a protecting group developed for the amino group, such as carbamates and amides, an example being a BOC protecting group. Such protecting groups are well known and appreciated in the art. The reagents and starting materials are generally readily available to one of ordinary skill in the art. Others may be made by standard techniques of organic and heterocyclic chemistry which are analogous to the syntheses of known structurally similar compounds and the procedures described in the Preparations and Examples which follow including any novel procedures. Intermediates and processes useful for the synthesis of the compounds of formula (I), (II), (IIA) or (III) are intended to be included in this description.

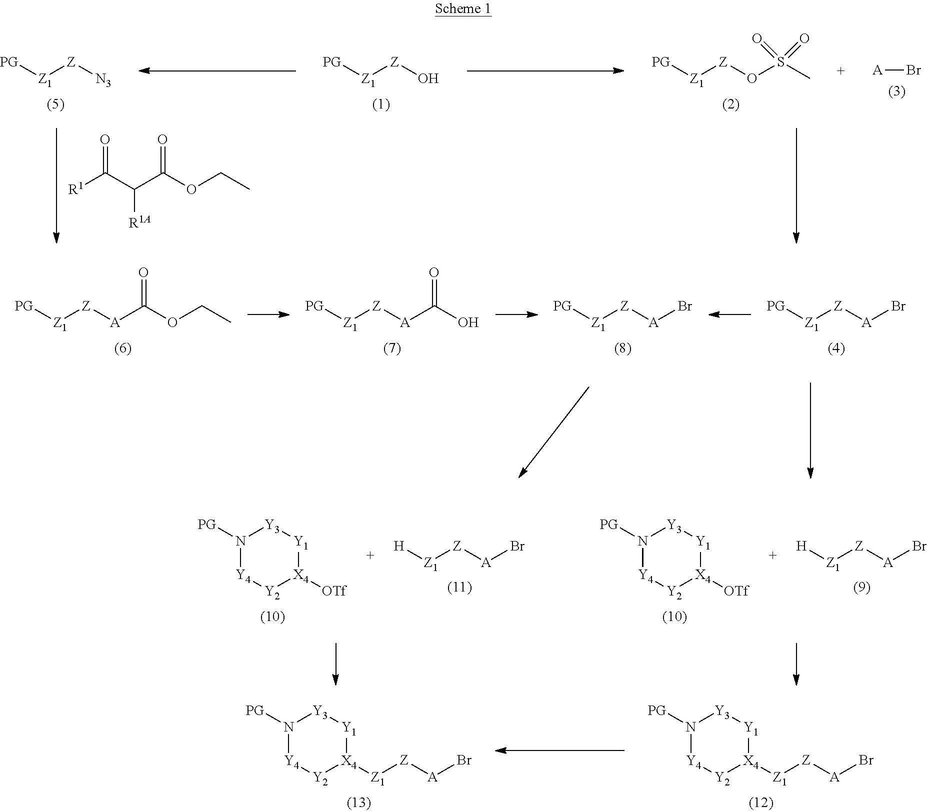

Scheme 1 depicts the preparation of compounds of (4) and (8) that lead to compound 13 through different routes and will be further elaborated to formula (I). A person of skill in the art will recognize that alcohol (1) may react with a mesyl chloride to afford mesylate (2) that can be further reacted with (3) to provide (4). Treatment of compound (4) with LDA and an appropriate alkylating agent may afford compound (8). Alternatively, one skilled in the art may start with compound (3) that is substituted bromide (3) that when reacted with mesylate (2) directly affords compounds of (8).

Compound (3) can also be synthesized through an alternative route as depicted in Scheme 1. Additionally, a skilled artisan will appreciate that alcohol (1) may react under Mitsunobu conditions to provide azide (5). Azide (5) may be condensed with a beta-keto ester to afford triazole ester (6) that can undergo saponification to provide carboxylic acid (7). Treatment of carboxylic acid (7) with bromine in the presence of base affords compounds of (8).

Scheme 1 further depicts the preparation of compounds of (12) and (13) that will be further elaborated to formula (I). A skilled artisan will recognize that compounds of (4) and (8) may be deprotected to give (9) and (11) that when reacted with triflate (10) result in bromide compounds of (12) and (13). Compound (12) may further be alkylated to give compounds of (13). A skilled artisan will appreciate that ketone compounds may be substituted for the triflate compounds of (10). Alternatively, reacting compound (9) or (11) with an appropriate ketone under reductive amination conditions may also afford compounds (12) or (13).

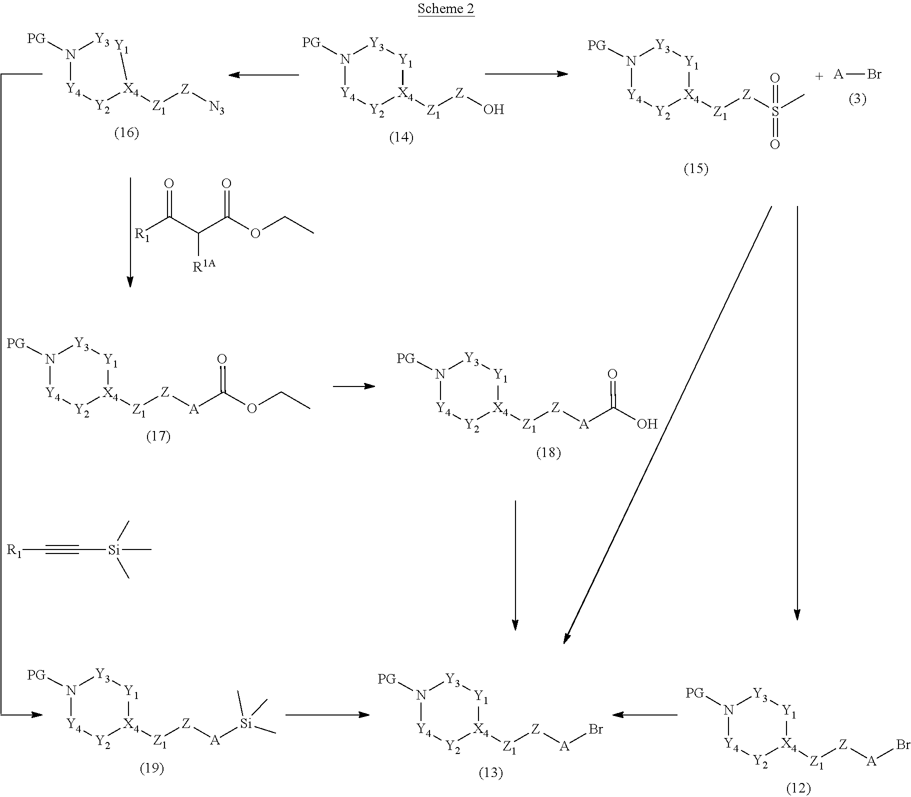

Scheme 2 depicts an alternative preparation of compounds of (12) and (13) that will be further elaborated to formula (I). A person of skill in the art will recognize that alcohol (14) may react with mesyl chloride to afford mesylate (15) that can be further reacted with a compound (3) to provide (12). Compounds of (12) may further be alkylated to furnish compounds of (13). Alternatively, compound (3) that is substituted may be reacted with (15) to directly give compounds of (13).

Additionally, compound (13) may also be synthesized from alternative routes as depicted in Scheme 2. Alcohol (14) may react under Mitsunobu conditions to provide azide (16). Azide (16) may be condensed with a beta-keto ester to afford triazole ester (17) that can undergo saponification to provide carboxylic acid (18). Treatment of carboxylic acid (18) with bromine in the presence of base affords bromide (13). Alternatively, azide (16) may be reacted with an appropriately substituted trimethylsilylalkyne to afford trimethyl silyl analog (19). Reaction of (19) with NBS in the presence of silicon dioxide provides compounds of (13).

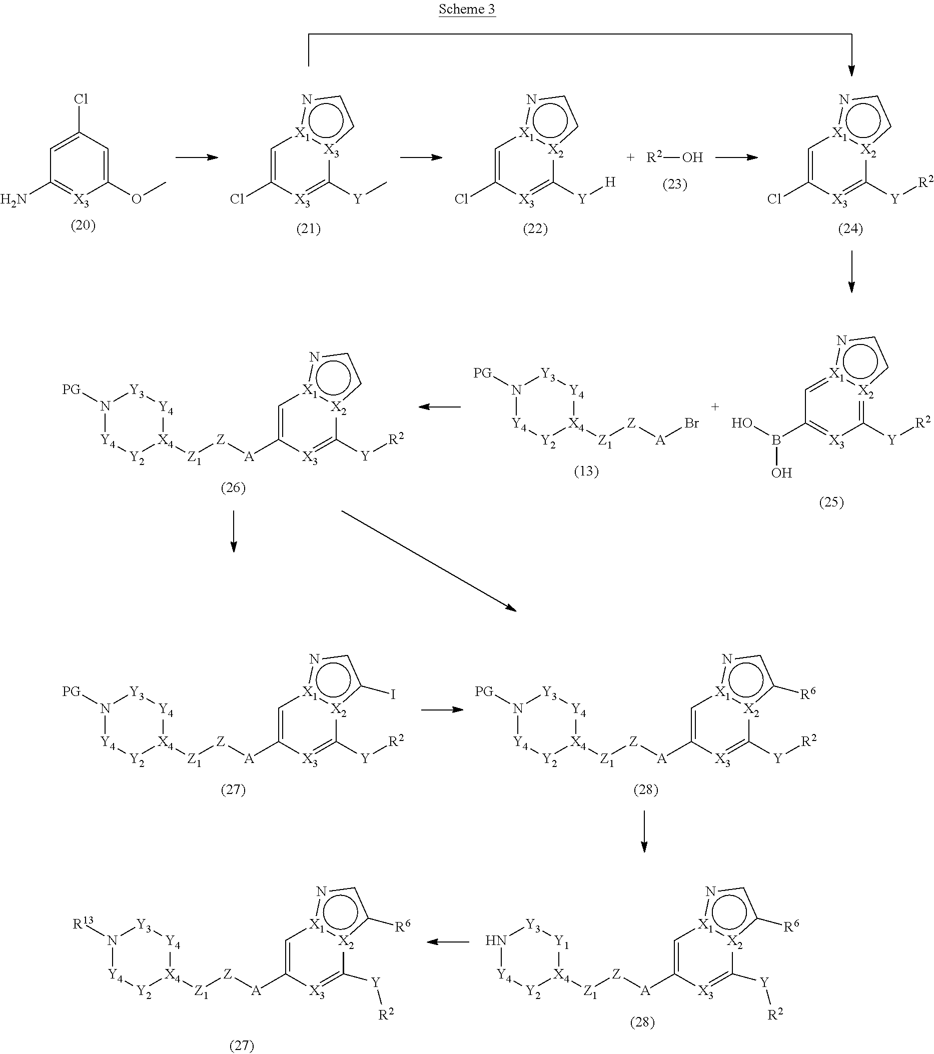

Scheme 3 depicts the preparation of compounds of formula (I). Reaction of (20) with chloroacetaldehyde provides heteroaryl chloride (21) that may be dem ethylated to give (22). Compound (22) may react with alcohol (23) via Mitsunobu conditions to provide a R² substituted heteroaryl chloride (24). A skilled artisan will recognize that when R² is methyl, it is unnecessary to demethylate (22) to arrive at a R² substituted (24). Conversion of heteroaryl chloride (24) to boronic acid (25) is achieved by treatment of (24) with bis(pinocolato)diboron under palladium catalyzed conditions. Boronic acid (25) may be reacted with bromide (13) to result in compounds of (26) through Suzuki coupling. Iodination of compounds of (26) in the presence of NIS provides (27) that may be further elaborated to provide R⁶ substituted compounds of (28). Deprotection of (28) followed by reaction with R¹³-halogen results in compounds of formula (I).

Alternatively, as would be known to one skilled in the art, compounds of (26) may be reacted with reagents such as NCS or NBS to afford R⁶ as chlorine or bromine directly without the intermediate iodination step to arrive at (28). Compounds of (28) are deprotected to provide compounds of (29). Reaction with R¹³-halogen, results in compounds of formula (I).

Alternatively, a skilled artisan will appreciate that prior to conversion of (24) to boronic acid (25), compounds of (24) may first be iodinated then reacted with copper cyanide in DMF to afford a R⁶ substituted heteroaryl chloride (24). Next, the Re substituted heteroaryl chloride (24) may be converted to a R⁶ substituted boronic acid Re substituted (25) that can be reacted with bromide (13) to provide compounds of (28). Deprotection of (28) followed by reaction with R¹³-halogen results in compounds of formula (I).

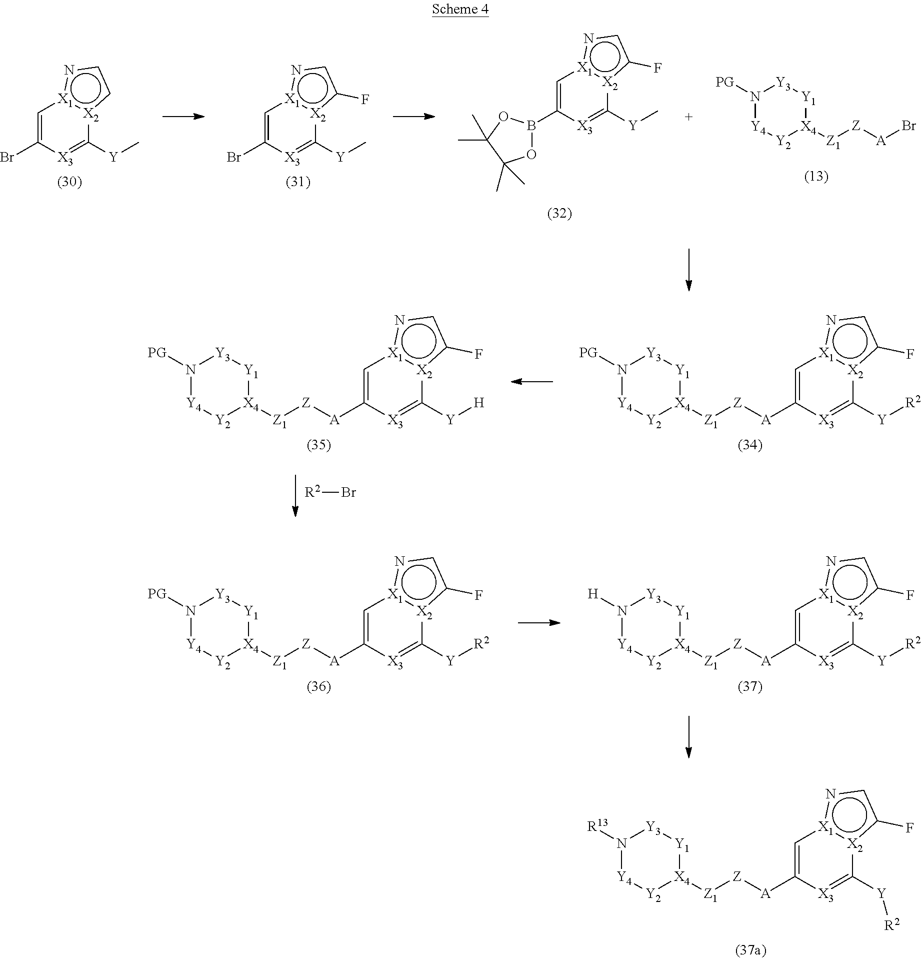

Scheme 4 depicts the preparation of compounds of (37a). Reaction of (30) in the presence of 1-(chloromethyl)-4-fluoro-1,4-diazabicyclo[2.2.2]octane-1,4-diium tetrafluoroborate affords the fluorinated bicyclic analog (31). Reaction of (31) with bis(pinocolato)diboron under palladium catalyzed conditions provides the boronate ester (32). Reaction of (32) with bromide (13) under palladium catalyzed conditions affords (34). Demethylation of (34) with aqueous sodium hydroxide and dodecane-1-thiol provides hydroxy bicyclic analog (35). Alkylation of (35) with the appropriate bromide gives compound (36). Deprotection of (36) followed by reaction with R¹³-halogen results in compound (37a).

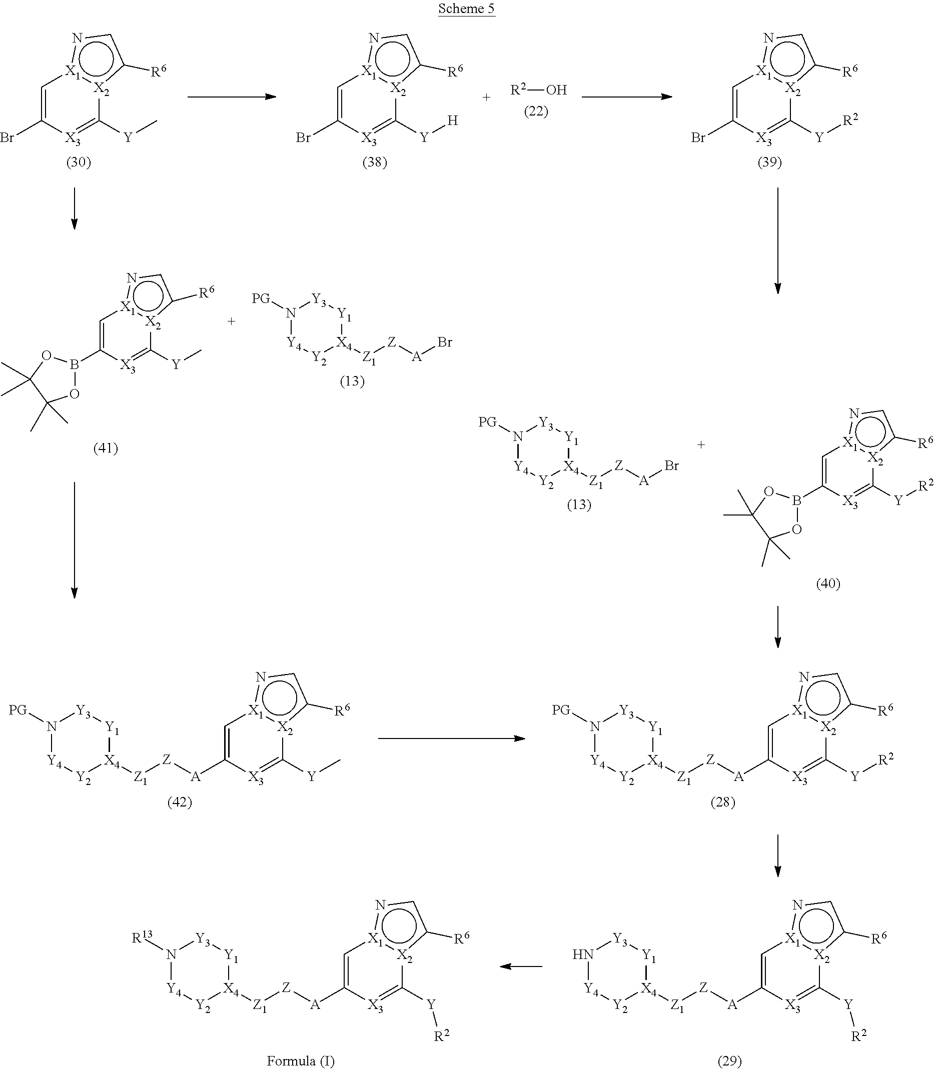

Scheme 5 depicts an alternative preparation of cyanoamino compounds of Formula (I). One of ordinary skill will recognize that compounds of (30) may be demethylated to afford (38). Demethylated (38) may be reacted with alcohol (22) under Mitsunobu conditions to provide R² substituted bromide compounds (39). Alternatively, Compound (39) may be alkylated by substituting R²OH (22) with the corresponding iodo analog R²I of (22). Conversion of R² substituted bromide (39) to boronate ester (40) may be achieved by treating (39) with bis(pinocolato)diboron under palladium catalyzed conditions. Boronate ester (40) reacted with (13) results in compounds of (28) through Suzuki coupling. Alternatively, a skilled artisan will recognize that, as depicted in Scheme 5, methylated compounds of (30) may first be treated with bis(pinocolato)diboron under palladium catalyzed conditions to provide boronate ester (41). Boronate ester (41) may be reacted with (13) to give compounds of (42) through Suzuki coupling. A skilled artisan will recognize that compounds of (42) are also compounds of (28) where R² is methyl. Lastly, compounds of (28) may be deprotected to provide amine (29). Reaction with R¹³-halogen results in compounds of formula (I).

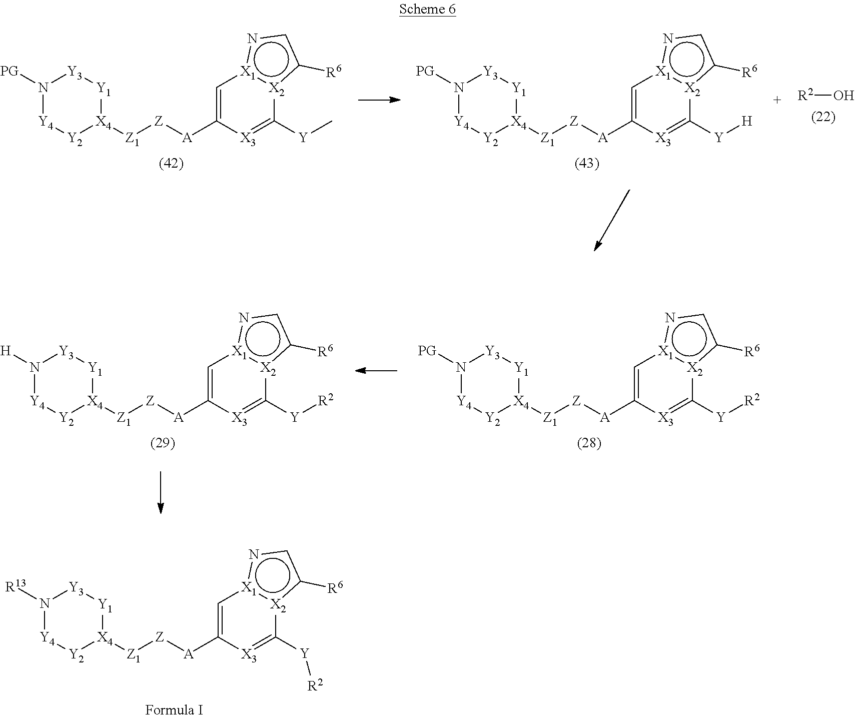

Formula (G) Scheme 6 depicts an alternative preparation of compounds of formula (I) starting with compounds (42) from Scheme 5 and compounds (28) from Schemes 3 and 5 where R² is methyl. Compounds (42) are demethylated to provide compounds of (43) that are reacted with alcohol (22) under Mitsunobu conditions to provide R² substituted compounds of (28) where the R² group is not methyl. R² substituted (28) may be deprotected to give compound (29). Reaction of compound (29) with R¹³-halogen results in compounds of formula (I).

One skilled in the art will recognize alternative reactants may also result in compounds of (28). For instance, alcohol (22) may be converted to triflate analog (R²-OTf) of (22) that may then be reacted with compounds of (43) to give compounds of (28). Additionally, alcohol compounds of (43) may first be converted to triflate compounds of (35) that may then be reacted with R²—NH2 analogs of (22) to afford an amine-R² substituted compounds of (28).

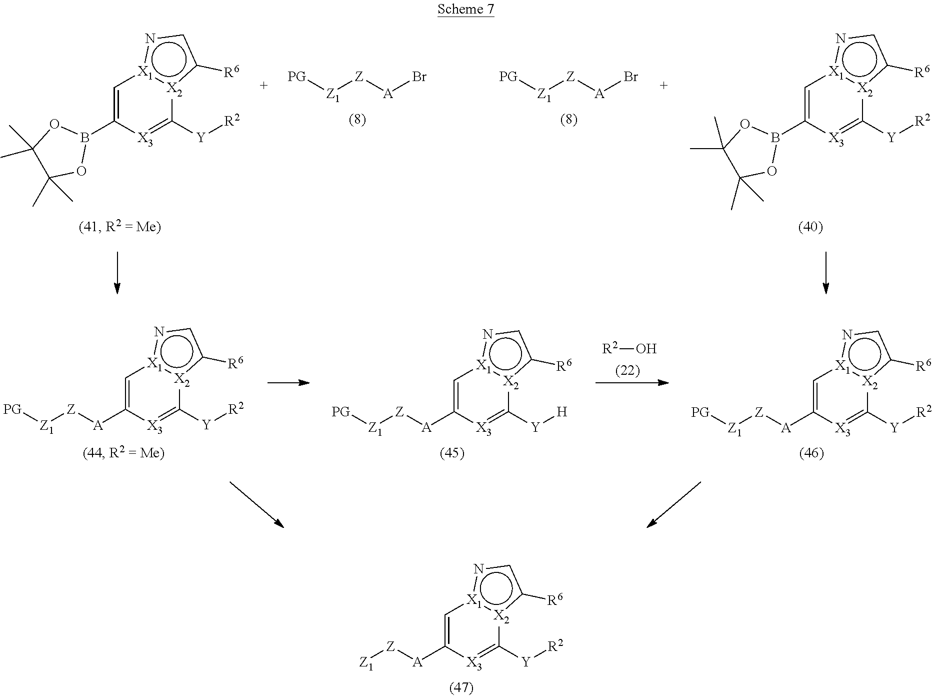

Scheme 7 depicts the preparation of compounds of (47) that are further elaborated to cyanoamino compounds of Formula (I) in Scheme 8. Boronate ester (40), as prepared in Scheme 5, may be reacted with (8) to result in compounds of (46) through Suzuki coupling.

Alternatively, a skilled artisan will recognize that boronate ester (41), as prepared in Scheme 5, may be reacted with (8) to give compounds of (44) through Suzuki coupling. A skilled artisan will recognize that boronic acid Re substituted (25) as prepared in Scheme 3 may be substituted for boronic esters (40) and (41) in these reactions. Compounds of (44) may be demethylated to afford compounds of (45) that when reacted with alcohol (22) under Mitsunobu conditions afford compounds of (46). Lastly, compounds of (44) or (46) may be deprotected to provide amine (47).

A skilled artisan will recognize that amine (47) may also be prepared by replacing the R¹ substituted compounds (8) with unsubstituted compounds (4) to result in amine (47) where the A group is unsubstituted.

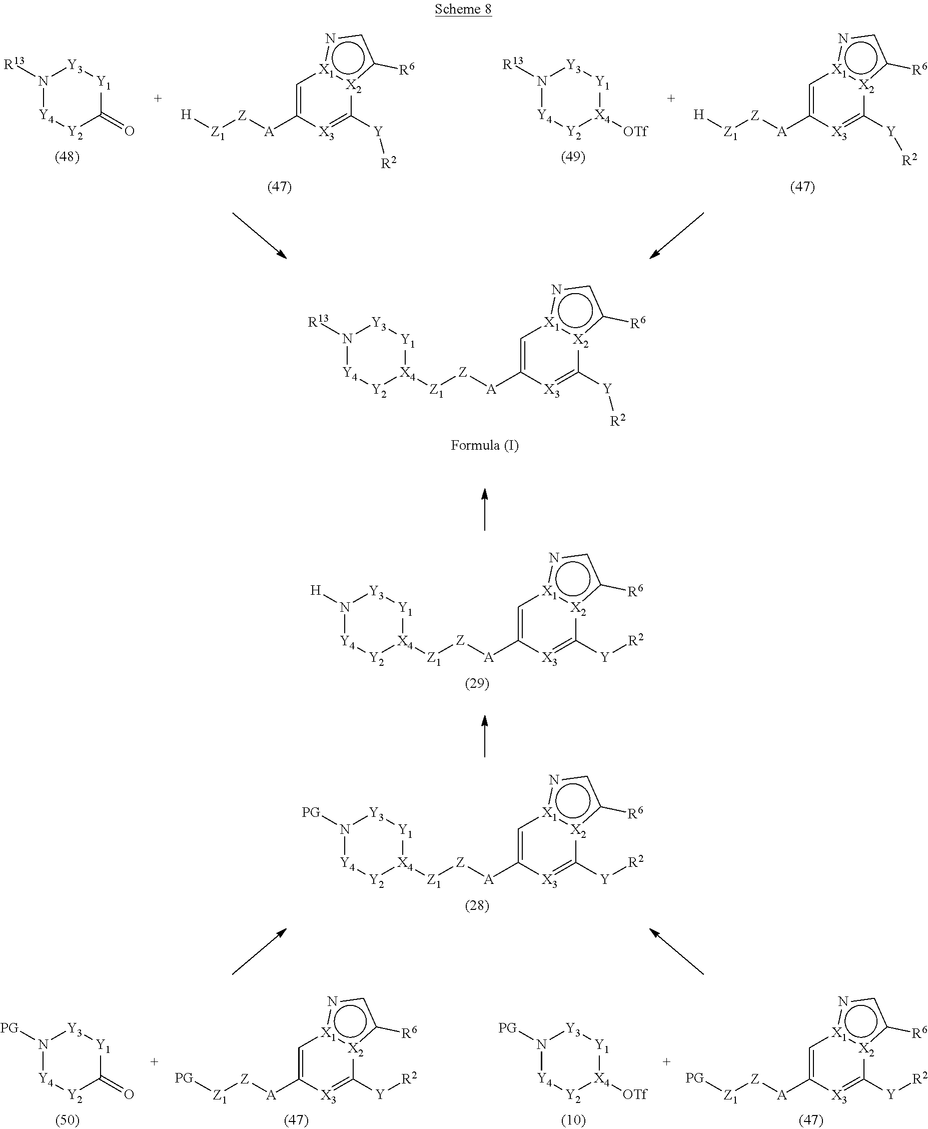

Scheme 8 depicts the preparation of compounds of Formula (I). Reductive amination of compounds of (47), as prepared in Scheme 7, with ketone (48) affords compounds of Formula (I). Alternatively, alkylation of (47) with triflate (49) gives compounds of Formula (I). A skilled artisan will also appreciate that mesylate (-OMs) compounds of (49) may be substituted for triflate (49). Additionally, a skilled artisan will recognize that N-protected ketone compounds of (50) and triflate compounds of (10) may also be used instead of compounds (48) and (49). Reductive amination of compounds of (47), as prepared in Scheme 7, with N-protected ketone (50) affords N-protected (28). Alkylation of compounds of (47) with N-protected triflate (10) also affords N-protected (28). Deprotected (28) can be reacted with R¹³-halogen to provide compounds of formula (I).

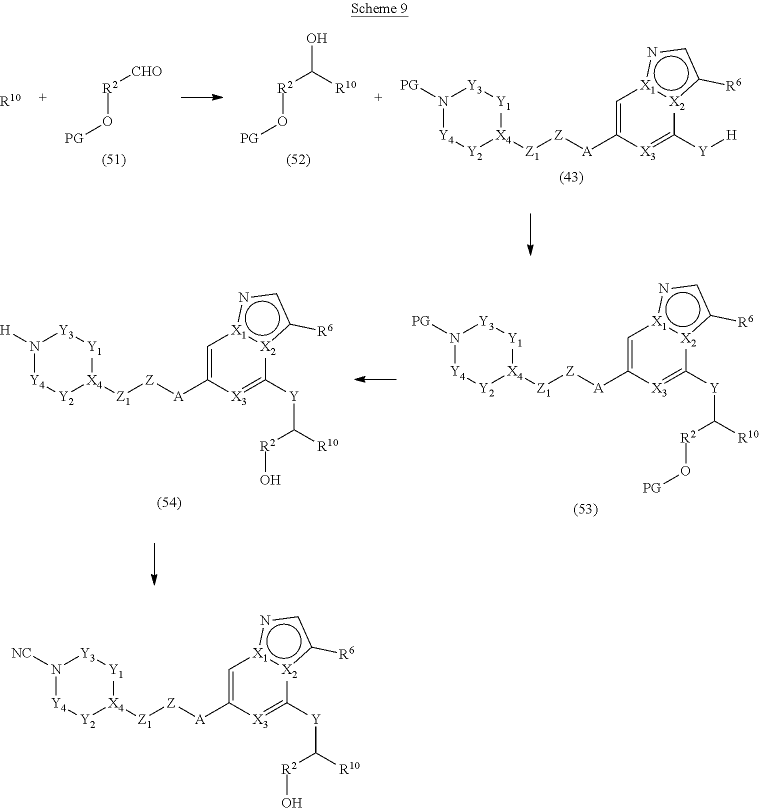

Scheme 9 depicts the preparation of compounds of (54a). Compounds of R¹⁰, where R¹⁰ is a 5-6 membered aryl or a 5-6-membered heteroaryl substituted with, but not limited to a halogen, may undergo a halogen-magnesium exchange with reagents such as iPrMgCl. The resultant Grignard reagent is reacted with the TBDMS protected alcohol R² aldehyde (51) to convert the aldehyde into the alcohol (52). Protecting groups other than TBDMS may be used to protect the R² alcohol in this step. Reacting the alcohol (52) under Mitsunobu conditions provides compounds (53). Treatment of (53) under acidic conditions affords the double deprotected compound (54). Reacting (54) with R¹³ halogen results in compounds of (54a).

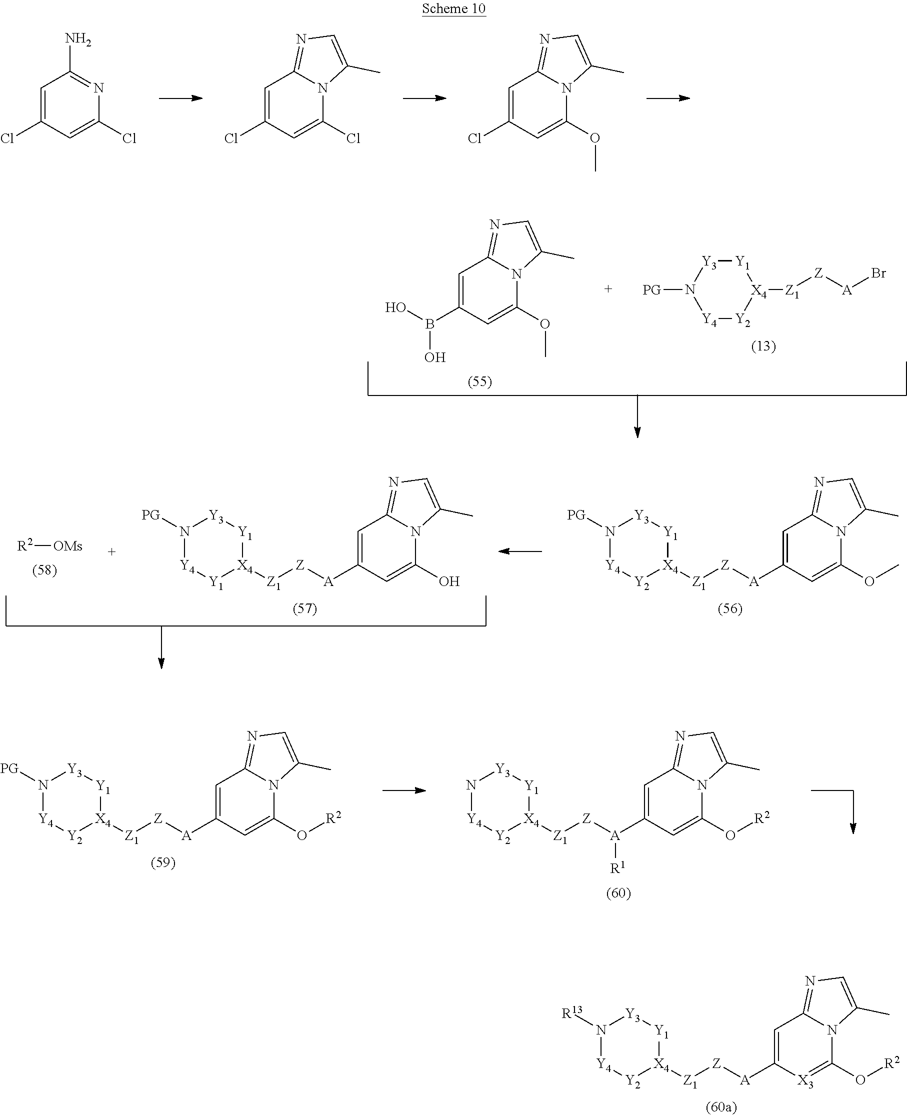

Scheme 10 depicts the preparation of compounds of (60a). 4,6-dichloropyridin-2-amine is reacted with a solution of 2-bromo-1,1-dimethoxypropane previously treated with aq. HCl in EtOH to afford 5,7-dichloro-3-methylimidazo[1,-a]pyridine. The 5,7-dichloro intermediate is treated with sodium methoxide to yield 7-chloro-5-methoxy-3-methylimidazo[1,-a]pyridine. The boronate ester (55) is formed by Pd catalyzed reaction of 7-chloro-5-methoxy-3-methylimidazo[1,-a]pyridine with bis(pinacolato)diboron. Suzuki coupling of boronate ester (55) with bromide (56) affords compound (57). Hydrolysis of (57) with NaOH gives hydroxy compound (58). Alkylation of (58) with mesylate (59) provides compound (60). Deprotection of (60) is reacted with R¹³-halogen to afford compounds of (60a).

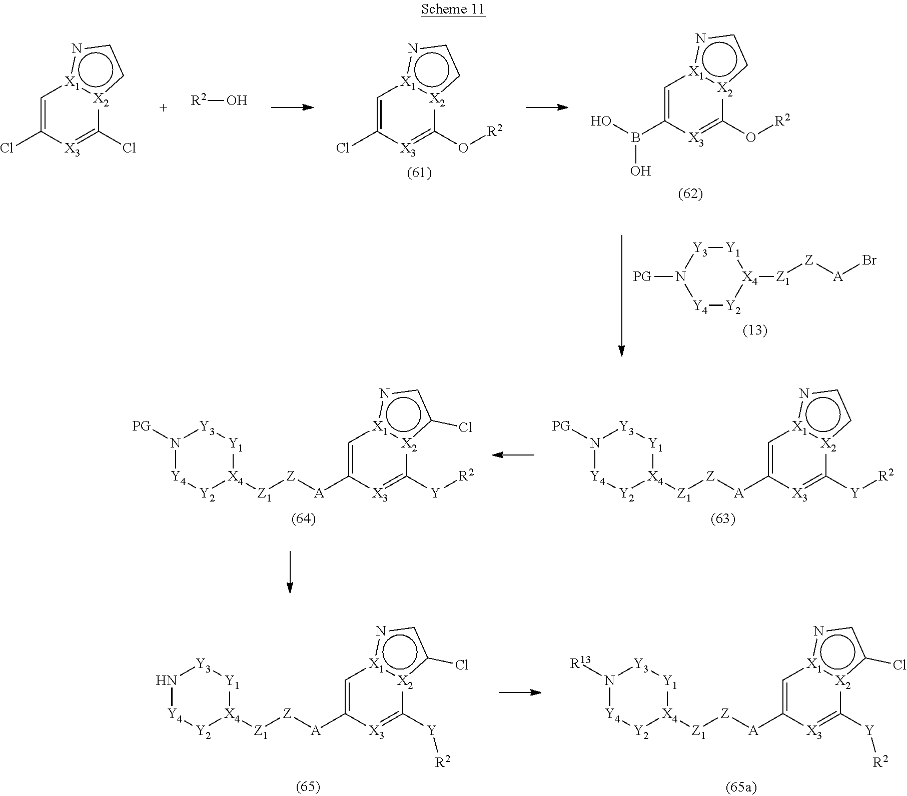

Scheme 11 depicts the preparation of (65a). Treatment of R²OH in the presence of base followed by the addition of 4,6-dichloropyridin-2-amine affords compound (61). The boronate ester (62) is formed by the Pd catalyzed reaction of compound (61) with bis(pinacolato)diboron. Suzuki coupling of boronate ester (62) and compound (13) provides compound (63). Chlorination of (63) with 1,3-dichloro-5, 5-dimethyl-imidazolidine-2,4-dione affords compound (64) which is then deprotected under acidic conditions to give compound (65). Subsequent treatment of (65) with R¹³-halogen affords compounds of (65a).

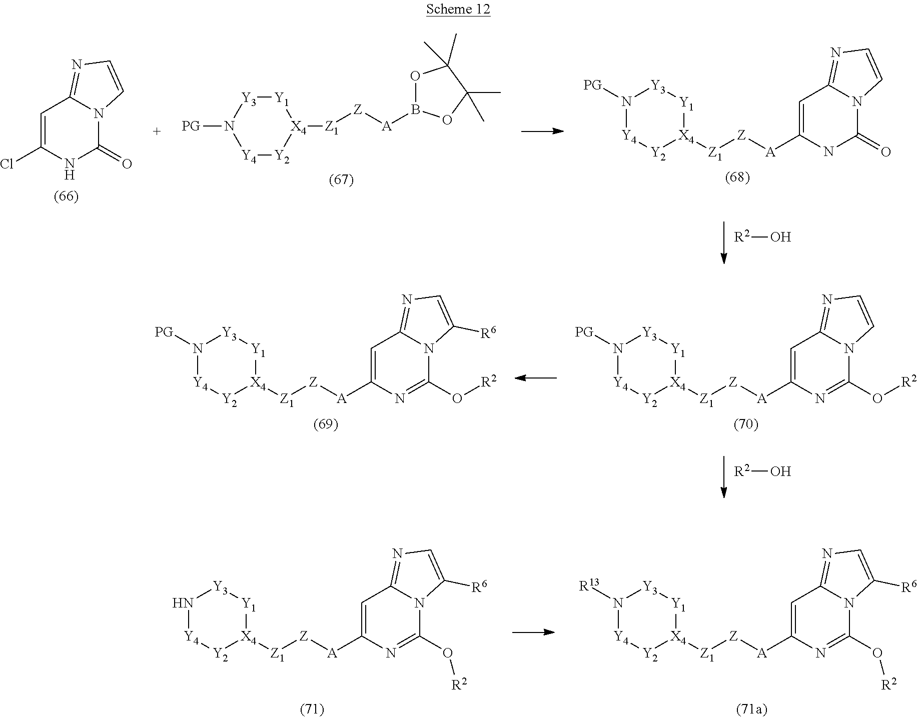

Scheme 12 depicts the preparation of (71a). Treatment of 7-chloro-6H-imidazo[1,-c]pyrimidin-5-one with the boronate ester (67) under palladium catalyzed conditions provides (68). Reaction of (68) under Mitsunobu conditions affords (70). Halogenation of (70) in the presence of NCS yields (69). Deprotection of (69) affords (71). Subsequent treatment of (71) with R¹³-halogen affords compounds of (71a).

The compounds of the present invention, or pharmaceutically acceptable salts thereof, may be prepared according to the following Preparations and Examples by methods well known and appreciated in the art. Suitable reaction conditions for the steps of these Preparations and Examples are well known in the art and appropriate substitutions of solvents and co-reagents are within the skill of the art. Likewise, it will be appreciated by those skilled in the art that synthetic intermediates may be isolated and/or purified by various well known techniques as needed or desired, and that frequently, it will be possible to use various intermediates directly in subsequent synthetic steps with little or no purification. As an illustration, compounds of the preparations and examples can be isolated, for example, by silica gel purification, isolated directly by filtration, or crystallization. Furthermore, the skilled artisan will appreciate that in some circumstances, the order in which moieties are introduced is not critical. The particular order of steps required to produce the compounds of the present invention is dependent upon the particular compound being synthesized, the starting compound, and the relative liability of the substituted moieties, as is well appreciated by the skilled chemist. All substituents, unless otherwise indicated, are as previously defined, and all reagents are well known and appreciated in the art.

### PREPARATIONS AND EXAMPLES

### Examples 1 & 2

### 4-[(1S)-1-(5-fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile (Example 1) and 4-[(1R)-1-(5-fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile (Example 2)

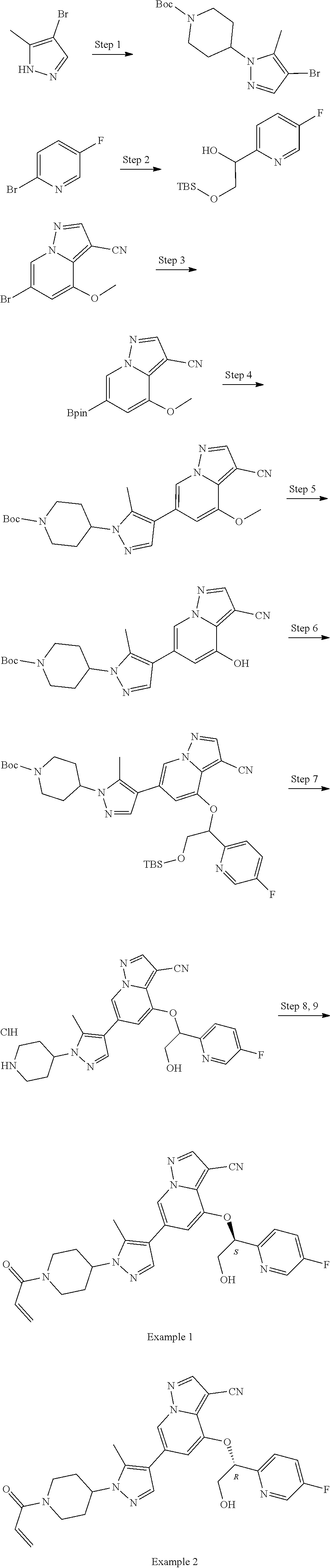

### Step 1. tert-Butyl 4-(4-bromo-5-methyl-pyrazol-1-yl) piperidine-1-carboxylate

To a solution of 4-bromo-5-methyl-1H-pyrazole (15.0 g, 93.17 mmol, 1.0 eq) in DMA (200 mL) was added NaH (7.45 g, 186.35 mmol, 2.0 eq) at 0° C. The reaction mixture was stirred for 0.5 h at 0° C. tert-Butyl 4-methylsulfonyloxypiperidine-1-carboxylate (39.04 g, 139.75 mmol, 1.5 eq) in DMA (50 mL) was added and the reaction mixture was stirred for 16 h at 70° C. After cooled to room temperature, the mixture was poured into a mixed solution of saturated NH₄Cl aqueous (150 mL) and EtOAc (400 mL). The organic layer was separated, and the aqueous was extracted with EA (150 mL). The combined organic layer was washed with brine (300 mL), dried over Na₂SO₄ and concentrated. The crude product was purified by silica gel column using (DCM/EtOAc=12/1) to give a yellow oil (11.4 g, 36% yield). LCMS m/z=344.3 (M+1).

### Step 2. 2-[tert-Butyl(dimethyl)silyl]oxy-1-(5-fluoro-2-pyridyl)ethanol

To a solution of 2-bromo-5-fluoro-pyridine (10.0 g, 56.82 mmol, 1.0 eq) in toluene (150 mL) was added i-PrMgCl (56.82 mL, 113.65 mmol, 2.0 eq) at −78° C. under N₂ protection. The mixture was stirred for 2 h at the same temperature. Then 2-[tert-butyl(dimethyl)silyl]-oxyacetaldehyde (19.81 g, 113.65 mmol, 2.0 eq) in toluene (50 mL) was added dropwise at −78° C. under N₂ protection. The mixture was stirred for 2 h at rt. The reaction was quenched with saturated aqueous of NH₄Cl (100 mL), extracted with EtOAc (200 mL×2). The combined organic layers were washed with brine (200 mL), dried over Na₂SO₄ and concentrated. The crude product was purified by silica gel column (Petroleum ether/Ethyl acetate=5/1) to give a yellow oil (12.8 g, 83% yield). LCMS m/z=272.5 (M+1).

### Step 3. 4-Methoxy-6-(4,4,5,5-tetramethyl-1,3,2-dioxaborolan-2-yl)pyrazolo[1,5-a]pyridine-3-carbonitrile

To a solution of 6-bromo-4-methoxy-pyrazolo[1,5-a]pyridine-3-carbonitrile (15.0 g, 59.51 mmol, 1.0 eq) in dioxane (150 mL) was added KOAc (11.66 g, 119.02 mmol, 2.0 eq), BPD (45.35 g, 178.52 mmol, 3.0 eq) and Pd(dppf)Cl₂ (2.43 g, 2.98 mmol, 0.1 eq). The reaction mixture was stirred for 4 h at 90° C. under N2 protection. After cooled to room temperature, the reaction mixture was concentrated. The crude product was purified by silica gel column (Petroleum ether/Ethyl acetate=1/1) to give a yellow solid (15.2 g, 85. yield). LCMS m/z=300.2 (M+1).

### Step 4. tert-Butyl 4-[4-(3-cyano-4-methoxy-pyrazolo[1,5-a]pyridin-6-yl)-5-methyl-pyrazol-1-yl]piperidine-1-carboxylate

To a solution of 4-methoxy-6-(4,4,5,5-tetramethyl-1,3,2-dioxaborolan-2-yl)pyrazolo[1,5-a]pyridine-3-carbonitrile (11.4 g, 33.12 mmol, 1.0 eq) and tert-butyl 4-(4-bromo-5-methyl-pyrazol-1-yl) piperidine-1-carboxylate (12.88 g, 43.05 mmol, 1.3 eq) in dioxane (150 mL) and water (15 mL) were added K₂CO₃ (9.14 g, 66.23 mmol, 2.0 eq) and Pd(dppf)Cl₂ (1.35 g, 1.66 mmol, 0.05 eq). The reaction mixture was stirred for 4 h at 100° C. under N2 protection. After cooled to room temperature, the reaction mixture was concentrated. The crude product was purified by silica gel column (Petroleum ether/Ethyl acetate=2/1) to give a yellow solid (6.50 g, 45% yield). LCMS m/z=(M+1).

### Step 5. tert-Butyl 4-[4-(3-cyano-4-hydroxy-pyrazolo[1,5-a]pyridin-6-yl)-5-methyl-pyrazol-1-yl]piperidine-1-carboxylate

To a solution of tert-butyl 4-[4-(3-cyano-4-methoxy-pyrazolo[1,5-a]pyridin-6-yl)-5-methyl-pyrazol-1-yl]piperidine-1-carboxylate (6.4 g, 14.66 mmol, 1.0 eq) in DMA (130 mL) and water (7 mL) was added NDM (8.89 g, 43.99 mmol, 3.0 eq) and NaOH (1.76 g, 43.99 mmol, 3.0 eq). The reaction mixture was stirred for 3 h at 60° C. After cooled to room temperature, the mixture was diluted with water, acidified with FA (pH=4), and extracted with EA (100 mL×2). The combined organic layers were washed with brine, dried over Na₂SO₄, and concentrated under reduced pressure. The residue was crystallized with MTBE:DCM:MeOH (100:10:1, 50 mL). The precipitated solids were collected by filtration and wash with hexane, concentrated in vacuum to give a yellow solid (5.2 g, 84% yield). LCMS m/z=423.6 (M+1).

### Step 6. tert-Butyl 4-[4-[4-[2-[tert-butyl(dimethyl)silyl]oxy-1-(5-fluoro-2-pyridyl) ethoxy]-3-cyano-pyrazolo[1,5-a]pyridin-6-yl]-5-methyl-pyrazol-1-yl]piperidine-1-carboxylate

To a solution of tert-butyl 4-[4-(3-cyano-4-hydroxy-pyrazolo[1,5-a]pyridin-6-yl)-5-methyl-pyrazol-1-yl]piperidine-1-carboxylate (1.5 g, 3.55 mmol, 1.0 eq), 2-[tert-butyl(dimethyl)-silyl]oxy-1-(5-fluoro-2-pyridyl)ethanol (1.44 g, 5.33 mmol, 1.5 eq) and PPh₃ (18.6 g, 71.0 mmol, 20.0 eq) in THF (75 mL), then DIAD (5.74 g, 28.4 mmol, 8.0 eq) in THF (10 mL) was added dropwise at 0° C. The reaction mixture was stirred for 2 h at rt. The solvent was concentrated, and the crude product was purified by silica gel column (Petroleum Ether/EtOAc=2/1) to give a yellow solid (2.0 g, 83% yield). LCMS m/z=676.4 (M+1).

### Step 7. 4-[1-(5-Fluoro-2-pyridyl)-2-hydroxy-ethoxy]-6-[5-methyl-1-(4-piperidyl) pyrazol-4-yl]pyrazolo[1,5-a]pyridine-3-carbonitrile hydrochloride

To a solution of tert-butyl 4-[4-[4-[2-[tert-butyl(dimethyl)silyl]oxy-1-(5-fluoro-2-pyridyl)ethoxy]-3-cyano-pyrazolo[1,5-a]pyridin-6-yl]-5-methyl-pyrazol-1-yl]piperidine-1-carboxylate (2.0 g, 2.96 mmol, 1.0 eq) in DCM (20 mL) was added HCl/EA (4 M, 10 mL). The reaction mixture was stirred for 2 h at rt. The reaction mixture was concentrated in vacuum to give a yellow solid as the HCl salt (1.4 g). LCMS m/z=462.6 (M+1).

### Step 8. 4-[1-(5-Fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile

To a solution of 4-[1-(5-fluoro-2-pyridyl)-2-hydroxy-ethoxy]-6-[5-methyl-1-(4-piperidyl)pyrazol-4-yl]pyrazolo[1,5-a]pyridine-3-carbonitrile hydrochloride (400 mg, 0.803 mmol, 1.0 eq) and DIEA (1.04 g, 8.03 mmol, 10.0 eq) in DCM (8 mL) was added prop-2-enoyl chloride (73 mg, 0.803 mmol, 1.0 eq) at 0° C. The resulting mixture was stirred at rt for 2 h under N₂ protection. After the reaction was completed, the mixture was quenched with water (10 mL) and extracted with DCM (20 mL×2). The combined organic layer was dried over Na₂SO₄ and concentrated. The residue was purified by silica gel flash column chromatography (DCM/MeOH=20/1) to afford a crude product, that was further purified by Prep-HPLC (Prep-C18, 5 μM Triart column, 20×150 mm, YMC-Actus; gradient elution of 40% MeCN in water to 60% MeCN in water over a 10 min period, where both solvents contain 0.05% NH₃·H₂O) to give a yellow solid (135 mg, 33% yield). LCMS m/z=516.3 (M+1).

### Step 9. Example 1 and Example 2

4-[1-(5-Fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile was separated by SFC (MeOH, 50% DCM, 20 mM NH3 Cellulose-SC) to give Example 1 4-[(1S)-1-(5-fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile as a yellow solid (26.3 mg); LCMS m/z=516.3 (M+1); 1H-NMR (400 MHz, DMSO-d6) δ 8.62-8.58 (m, 2H), 8.51 (d, J=1.2 Hz, 1H), 7.82-7.73 (m, 2H), 7.71 (s, 1H), 7.07 (s, 1H), 6.87 (dd, J=16.8 Hz, 10.4 Hz, 1H), 6.13 (dd, J=16.8 Hz, 2.4 Hz, 1H), 5.83-5.81 (m, 1H), 5.69 (dd, J=10.4 Hz, 2.4 Hz, 1H), 5.12 (t, J=5.6 Hz, 1H), 4.60-4.49 (m, 2H), 4.24-4.14 (m, 1H), 4.02-3.91 (m, 2H), 3.29-3.20 (m, 1H), 2.85 (t, J=11.6 Hz, 1H), 2.32 (s, 3H), 1.96-1.74 (m, 4H); and Example 2 4-[(1R)-1-(5-fluoro-2-pyridyl)-2-hydroxy-ethoxy]-6-[5-methyl-1-(1-prop-2-enoyl-4-piperidyl)pyrazol-4-yl]pyrazolo[1,5-a]pyridine-3-carbonitrile as a yellow solid (27.4 mg); LCMS m/z=516.3 (M+1); 1H-NMR (400 MHz, DMSO-d6) δ 8.63-8.57 (m, 2H), 8.51 (s, 1H), 7.83-7.74 (m, 2H), 7.71 (s, 1H), 7.08 (s, 1H), 6.86 (dd, J=16.8 Hz, 10.4 Hz, 1H), 6.13 (dd, J=16.8 Hz, 2.4 Hz, 1H), 5.86-5.80 (m, 1H), 5.69 (dd, J=10.4 Hz, 2.4 Hz, 1H), 5.12 (t, J=5.6 Hz, 1H), 4.61-4.48 (m, 2H), 4.19 (d, J=13.2 Hz, 1H), 4.03-3.91 (m, 2H), 3.30-3.20 (m, 1H), 2.92-2.79 (m, 1H), 2.32 (s, 3H), 1.98-1.76 (m, 4H).

### Example 3. (R)-6-(1-(7-acryloyl-7-azaspiro[3.5]nonan-2-yl)-5-methyl-1H-1,2,3-triazol-4-yl)-4-(1-(pyridin-2-yl)ethoxy)pyrazolo[1,5-a]pyridine-3-carbonitrile

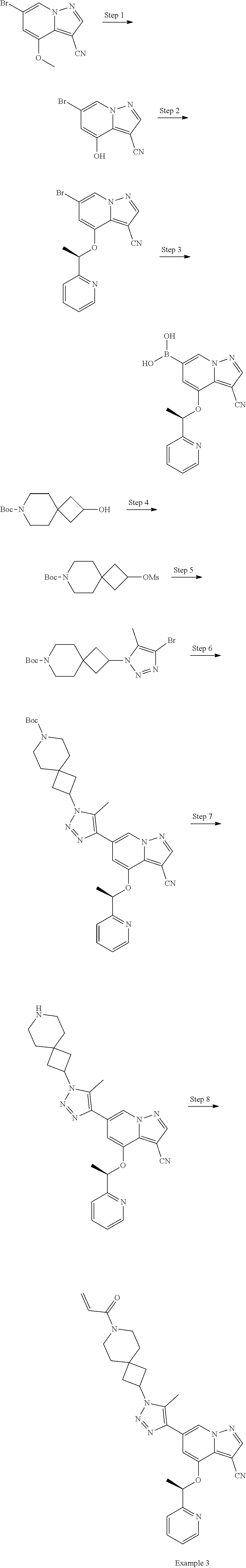

### Step 1. 6-bromo-4-hydroxy-pyrazolo[1,5-a]pyridine-3-carbonitrile

To a solution of 6-bromo-4-methoxy-pyrazolo[1,5-a]pyridine-3-carbonitrile (10.0 g, 39.8 mmol, 1.0 eq) and NDM (24.1 g, 119 mmol, 3.0 eq) in DMA (150 mL) was added a 50% aqueous NaOH solution (4.78 g, 119 mmol, 3.0 eq). The reaction mixture was stirred at 60° C. for 3 h under nitrogen. After the reaction was complete, the mixture was diluted with H₂O (100 mL), and then the pH of the solution was adjusted to 5-6 with aqueous HCOOH. The aqueous layer was extracted with EtOAc (300 mL) and washed with H₂O (100 mL×3). The organic layer was dried over Na₂SO₄ and concentrated in vacuo. The crude product was purified by silica gel flash column chromatography (Petroleum Ether/EtOAc=3/1) to give a brown solid (8.2 g, 87% yield). LCMS m/z=238.3 (M+1).

### Step 2. 6-bromo-4-[(1R)-1-(2-pyridyl)ethoxy]pyrazolo[1,5-a]pyridine-3-carbonitrile

To a solution of PPh₃ (8.29 g, 31.6 mmol, 1.5 eq) and DIAD (5.11 g, 25.3 mmol, 1.2 eq) in DCM (80 mL) was added 6-bromo-4-hydroxy-pyrazolo[1,5-a]pyridine-3-carbonitrile (5.0 g, 21.1 mmol, 1.0 eq) in DCM (30 mL) dropwise at −10° C. The solution was stirred for 20 min under nitrogen. Then (1S)-1-(2-pyridyl)ethanol (2.60 g, 21.1 mmol, 1.0 eq) in DCM (30 mL) was added at −10° C. and stirred at rt for 6 h. After the reaction was completed, the mixture was diluted with DCM (100 mL) and washed with H₂O (100 mL). The organic layer was dried over Na₂SO₄ and concentrated in vacuo. The crude product was purified by silica gel flash column chromatography (petroleum ether/EtOAc=1/1) to give a white solid (3.0 g, 42% yield). LCMS m/z=343.2 (M+1).

### Step 3. [3-cyano-4-[(1R)-1-(2-pyridyl)ethoxy]pyrazolo[1,5-a]pyridin-6-yl]boronic acid

To a solution of 6-bromo-4-[(1R)-1-(2-pyridyl)ethoxy]pyrazolo[1,5-a]pyridine-3-carbonitrile (1.0 g, 2.9 mmol, 1.0 eq) and BPD (1.5 g, 5.8 mmol, 2.0 eq) in dioxane (12 mL) was added KOAc (852 mg, 8.7 mmol, 3.0 eq) and Pd(PPh₃)₄ (173 mg, 0.15 mmol, 0.05 eq). The resulting mixture was stirred at 90° C. for 2 h under nitrogen. After the reaction was complete, the mixture was concentrated in vacuo. The crude product was purified by Prep-MPLC (Prep-C18, 20-45 μM, 120 g, Tianjin Bonna-Agela Technologies; gradient elution of 20% MeCN in water to 65% MeCN in water over a 7 min period, where both solvents contain 0.05% formic acid) to give a yellow solid (500 mg, 56% yield) as the formate salt. LCMS m/z=309.3 (M+1).

### Step 4. (7-methyl-7-azaspiro[3.5]nonan-2-yl) methanesulfonate

To a solution of tert-butyl 2-hydroxy-7-azaspiro[3.5]nonane-7-carboxylate (5.0 g, 20.72 mmol, 1.0 eq) and TEA (3.1 g, 31.08 mmol, 1.5 eq) in DCM (30 mL) was added MsCl (2.85 g, 24.86 mmol, 1.2 eq) at 0° C., and was stirred for 1 h. The reaction was diluted with DCM (50 ml) and washed with brine (20 mL). The organic layer was dried over Na₂SO₄ and concentrated to give a yellow solid (6.1 g, 92% yield). LCMS m/z=320.3 (M+1).

### Step 5. tert-butyl 2-(4-bromo-5-methyl-triazol-1-yl)-7-azaspiro[3.5]nonane-7-carboxylate

To solution of (7-methyl-7-azaspiro[3.5]nonan-2-yl) methanesulfonate (2.0 g, 6.26 mmol, 1.0 eq) and 4-bromo-5-methyl-1H-triazole (1.3 g, 8.14 mmol, 1.3 eq) in DMF (15 mL) was added Cs₂CO₃ (4.1 g, 12.52 mmol, 2.0 eq) and NaI (94 mg, 0.63 mmol, 0.1 eq). The mixture was stirred at 100° C. for 16 h and then was diluted with EtOAc (100 ml) and washed with brine (30 mL). The organic layer was dried over Na₂SO₄ and concentrated. The residue was purified by Prep-TLC (Petroleum Ether/EtOAc=3/1) to give a white solid (310 mg, 13% yield). LCMS m/z=385.3 (M+1).

### Step 6. tert-butyl 2-[4-[3-cyano-4-[(1R)-1-(2-pyridyl)ethoxy]pyrazolo[1,5-a]pyridin-6-yl]-5-methyl-triazol-1-yl]-7-azaspiro[3.5]nonane-7-carboxylate

To a solution of tert-butyl 2-(4-bromo-5-methyl-triazol-1-yl)-7-azaspiro[3.5]nonane-7-carboxylate (150 mg, 0.39 mmol, 1.0 eq) and [3-cyano-4-[(1R)-1-(2-pyridyl)ethoxy]pyrazolo-[1,5-a]pyridin-6-yl]boronic acid (179 mg, 0.39 mmol, 1.0 eq) in dioxane (6 ml) and H₂O (3 ml) was added K₂CO₃ (108 mg, 0.78 mmol, 2.0 eq) and Pd(dtbpf)Cl₂ (25 mg, 0.08 mmol, 0.1 eq). The mixture was stirred at 90° C. for 4 h under nitrogen, then was diluted with EtOAc (30 ml) and washed with brine (10 ml). The organic layer was dried over Na₂SO₄ and concentrated. The crude product was purified by Prep-TLC (Petroleum Ether/EtOAc=2/1) to give a yellow solid (165 mg, 75% yield). LCMS m/z=569.4 (M+1).

### Step 7. 6-[1-(7-azaspiro[3.5]nonan-2-yl)-5-methyl-triazol-4-yl]-4-[(1R)-1-(2-pyridyl)ethoxy]pyrazolo[1,5-a]pyridine-3-carbonitrile hydrochloride

A solution of tert-butyl 2-[4-[3-cyano-4-[(1R)-1-(2-pyridyl)ethoxy]pyrazolo[1,5-a]pyridin-6-yl]-5-methyl-triazol-1-yl]-7-azaspiro[3.5]nonane-7-carboxylate (155 mg, 0.27 mmol, 1.0 eq) in HCl/dioxane (4 ml) was stirred at rt for 2 h. After the reaction was completed, the reaction was concentrated to give a yellow solid (150 mg). LCMS m/z=(469.4).

### Step 8. (R)-6-(1-(7-acryloyl-7-azaspiro[3.5]nonan-2-yl)-5-methyl-1H-1,2,3-triazol-4-yl)-4-(1-(pyridin-2-yl)ethoxy)pyrazolo[1,5-a]pyridine-3-carbonitrile

To a solution of 6-[1-(7-azaspiro[3.5]nonan-2-yl)-5-methyl-triazol-4-yl]-4-[(1R)-1-(2-pyridyl)ethoxy]pyrazolo[1,5-a]pyridine-3-carbonitrile hydrochloride (140 mg, 0.30 mmol, 1.0 eq) and DIEA (140 mg, 1.50 mmol, 5.0 eq) in DCM (5 ml) was added dropwise prop-2-enoyl chloride (32 mg, 0.36 mmol, 1.2 eq) at 0° C. After stirring for 1 h, the reaction was diluted with DCM (20 ml) and washed with brine (10 ml). The organic layer was dried over Na₂SO₄ and concentrated. The crude product was purified by Prep-HPLC (Prep-C18, 5 μM Triart column, 20× 150 mm, YMC-Actus; gradient elution of 30% MeCN in water to 80% MeCN in water over a 12 min period, where both solvents contain 0.05% NH₃·H₂O) to a white solid (68 mg, 44% yield). LCMS m/z=523.4 (M+H); ¹H NMR (400 MHz, DMSO-d6) δ=8.66-8.64 (m, 2H), 8.59 (d, J=4.0 Hz, 1H), 7.84 (td, J=7.6 Hz, 2.0 Hz, 1H), 7.60 (d, J=8.0 Hz, 1H), 7.36-7.33 (m, 1H), 7.16 (s, 1H), 6.88-6.77 (m, 1H), 6.08 (d, J=16.8 Hz, 1H), 5.83 (q, J=6.8 Hz, 1H), 5.66 (d, J=10.4 Hz, 1H), 5.12-5.04 (m, 1H), 3.60-3.52 (m, 2H), 3.50-3.43 (m, 2H), 2.56-2.54 (m, 2H), 2.43-2.38 (m, 2H), 2.33 (s, 3H), 1.73-1.67 (m, 5H), 1.60-1.58 (m, 2H).

### Example 4. (R)-1-(2-(4-(3-chloro-4-(1-(pyridin-2-yl)ethoxy)-pyrazolo[1,5-a]pyridin-6-yl)-5-methyl-1H-1,2,3-triazol-1-yl)-7-azaspiro[3.5]nonan-7-yl)prop-2-en-1-one

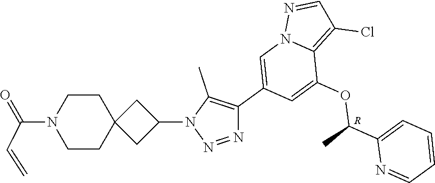

Example 4 was made by methods analogous to those described above.

LCMS m/z=532.4 (M+H); ¹H NMR (400 MHz, DMSO-d6) δ=8.58 (d, J=4.0 Hz, 1H), 8.42 (d, J=0.8 Hz, 1H), 8.12 (s, 1H), 7.84 (td, J=7.6 Hz, 1.6 Hz, 1H), 7.55 (d, J=8.0 Hz, 1H), 7.35-7.32 (m, 1H), 6.86-6.77 (m, 2H), 6.09 (d, J=16.4 Hz, 1H), 5.71 (q, J=6.4 Hz, 1H), 5.66 (d, J=10.4 Hz, 1H), 5.11-5.03 (m, 1H), 3.59-3.50 (m, 2H), 3.46-3.36 (m, 2H), 2.54-2.51 (m, 2H), 2.43-2.38 (m, 2H), 2.31 (s, 3H), 1.75-1.70 (m, 5H), 1.60-1.58 (m, 2H).

### Examples 5 & 6

### (R)-1-(2-(4-(3-chloro-4-(2-hydroxy-1-(pyridin-2-yl)ethoxy)pyrazolo[1,5-a]pyridin-6-yl)-5-methyl-1H-pyrazol-1-yl)-7-azaspiro[3.5]nonan-7-yl)prop-2-en-1-one (Example 5) and (S)-1-(2-(4-(3-chloro-4-(2-hydroxy-1-(pyridin-2-yl)ethoxy)pyrazolo[1,5-a]pyridin-6-yl)-5-methyl-1H-pyrazol-1-yl)-7-azaspiro[3.5]nonan-7-yl)prop-2-en-1-one (Example 6)

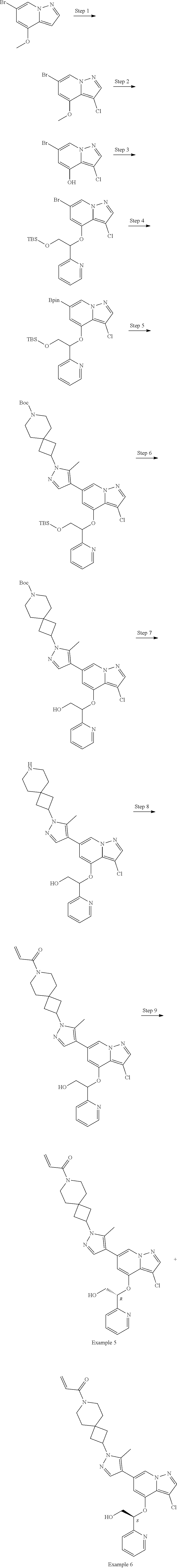

### Step 1. 6-Bromo-3-chloro-4-methoxy-pyrazolo[1,5-a]pyridine

To a solution of 6-bromo-4-methoxy-pyrazolo[1,5-a]pyridine (6.0 g, 26.4 mmol, 1.0 eq) in DCM (50 mL) was added solid NCS (3.5 g, 26.43 mmol, 1.0 eq). The resulting mixture was stirred at rt for 12 h, and then diluted with water (50 mL). The aqueous layer was extracted with DCM (50 mL×3). The combined organic layers were washed with brine (100 mL), dried over Na₂SO₄, and concentrated. The crude product was purified by silica gel chromatography eluted with (petroleum ether/EtOAc=8/1) to give a white solid (5.6 g, 81% yield). LCMS m/z=261.1 (M+1).

### Step 2. 6-bromo-3-chloro-pyrazolo[1,5-a]pyridin-4-ol

To a solution of 6-bromo-3-chloro-4-methoxy-pyrazolo[1,5-a](5.0 g, 19.12 mmol, 1.0 eq) in DMA (20 mL) was added NaOH (50% aq. solution) (3.82 g, 95.6 mmol, 5.0 eq) and NDM (11.59 g, 57.36 mmol, 3.0 eq) at rt. The resulting mixture was stirred at 100° C. for 3 h and was concentrated to remove the volatile solvent. The pH of the mixture was adjusted to 5 by addition of hydrochloric acid solution (1 M) and was extracted with EtOAc (30 mL×3). The combined organic layers were washed with brine (50 mL), dried over Na₂SO₄ and concentrated. The crude product was purified by silica gel chromatography eluted with (Petroleum ether/EtOAc=2/1) to give a white solid (4.1 g, 16.6 mmol, 87% yield). LCMS m/z=246.9 (M+1).

### Step 3. [2-(6-bromo-3-chloro-pyrazolo[1,5-a]pyridin-4-yl)oxy-2-(2-pyridyl)ethoxy]-silane

To a solution of 2-[tert-butyl(dimethyl)silyl]oxy-1-(2-pyridyl)ethanol (1.5 g, 5.92 mmol, 1.0 eq) in THF (50 mL) was added 6-bromo-3-chloro-pyrazolo[1,5-a]pyridin-4-ol (1.46 g, 5.92 mmol, 1.0 eq) and PPh₃ (2.34 g, 8.89 mmol, 1.5 eq) at 0° C. The resulting mixture was stirred at 0° C. for 5 min. Then DEAD (1.43 g, 7.10 mmol, 1.2 eq) was added, and the resulting mixture was stirred at 0° C. for 4 h. The mixture was then quenched with H₂O (20 mL) and extracted with EtOAc (50 mL×2). The combined organic layers were washed with brine (20 mL), dried over Na₂SO₄ and concentrated. The crude product was purified by silica gel column chromatography (Petroleum ether/EtOAc=1/1) to give a yellow solid (1.6 g, 56% yield). LCMS m/z=482.2 (M+1).

### Step 4. tert-butyl-[2-[3-chloro-6-(4,4,5,5-tetramethyl-1,3,2-dioxaborolan-2-yl)pyrazolo[1,5-a]pyridin-4-yl]oxy-2-(2-pyridyl)ethoxy]-dimethyl-silane

To a solution of [2-(6-bromo-3-chloro-pyrazolo[1,5-a]pyridin-4-yl)oxy-2-(2-pyridyl)ethoxy]silane (1.0 g, 2.07 mmol, 1.0 eq) and BPD (2.63 g, 10.35 mmol, 5.0 eq) in dioxane (20 mL) was added Pd(dppf)Cl₂ (85 mg, 0.10 mmol, 0.05 eq) and KOAc (609 mg, 6.21 mmol, 3.0 eq). The resulting mixture was stirred at 100° C. for 6 h, then was quenched with the saturated NH₄Cl solution (20 mL) and extracted with EtOAc (20 mL×2). The combined organic layers were washed with brine (20 mL), dried over Na₂SO₄ and concentrated. The crude product was purified by silica gel column chromatography (Petroleum ether/EtOAc=1/1) to give a yellow solid (830 mg, 76% yield). LCMS m/z=530.1 (M+1).

### Step 5. tert-butyl 2-[4-[4-[2-[tert-butyl(dimethyl)silyl]oxy-1-(2-pyridyl)ethoxy]-3-chloro-pyrazolo[1,5-a]pyridin-6-yl]-5-methyl-pyrazol-1-yl]-7-azaspiro[3.5]nonane-7-carboxylate

To a solution of tert-butyl 2-(4-bromo-5-methyl-pyrazol-1-yl)-7-azaspiro[3.5]nonane-7-carboxylate (500 mg, 1.30 mmol, 1.0 eq) and tert-butyl-[2-[3-chloro-6-(4,4,5,5-tetramethyl-1,3,2-dioxaborolan-2-yl)pyrazolo[1,5-a]pyridin-4-yl]oxy-2-(2-pyridyl)ethoxy]-dimethyl-silane (827 mg, 1.56 mmol, 1.2 eq) in dioxane (10 mL) and H₂O (1 mL) was added Pd(dppf)Cl₂ (53 mg, 0.65 mmol, 0.05 eq) and K₂CO₃ (539 mg, 3.90 mmol, 3.0 eq). The resulting mixture was stirred at 100° C. for 4 h. The mixture was quenched with saturated NH₄Cl (20 mL) and extracted with EtOAc (20 mL×2). The combined organic layers were washed with brine (10 mL), dried over Na₂SO₄ and concentrated. The crude product was purified by silica gel column chromatography (Petroleum ether/EtOAc=1/2) to give a yellow solid (660 mg, 71% yield). LCMS m/z=707.3 (M+1).

### Step 6. tert-butyl 2-[4-[3-chloro-4-[2-hydroxy-1-(2-pyridyl)ethoxy]pyrazolo[1,5-a]pyridin-6-yl]-5-methyl-pyrazol-1-yl]-7-azaspiro[3.5]nonane-7-carboxylate

To a solution of tert-butyl 2-[4-[4-[2-[tert-butyl(dimethyl)silyl]oxy-1-(2-pyridyl) ethoxy]-3-chloro-pyrazolo[1,5-a]pyridin-6-yl]-5-methyl-pyrazol-1-yl]-7-azaspiro[3.5]nonane-7-carboxylate (350 mg, 0.49 mmol, 1.0 eq) in THF (10 mL) was added TBAF (0.99 mL, 0.99 mmol, 2.0 eq, 1M in THF). The resulting mixture was stirred at rt for 4 h. The mixture was quenched with H₂O (10 mL) and extracted with EtOAc (10 mL×2). The combined organic layers were washed with brine (10 mL), dried over Na₂SO₄ and concentrated. The crude product was purified by silica gel column chromatography (Petroleum ether/EtOAc=1/2) to give a yellow solid (230 mg, 78% yield). LCMS m/z=593.5 (M+1).

### Step 7. 2-[6-[1-(7-azaspiro[3.5]nonan-2-yl)-5-methyl-pyrazol-4-yl]-3-chloro-pyrazolo[1,5-a]pyridin-4-yl]oxy-2-(2-pyridyl)ethanol HCl salt

To a solution of tert-butyl 2-[4-[3-chloro-4-[2-hydroxy-1-(2-pyridyl)ethoxy]pyrazolo-[1,5-a]pyridin-6-yl]-5-methyl-pyrazol-1-yl]-7-azaspiro[3.5]nonane-7-carboxylate (230 mg, 0.39 mmol, 1.0 eq) in DCM (6 mL) was added HCl (2 mL, 4M in EtOAc). The solution was stirred at rt for 4 h and was concentrated to give an off-white solid (240 mg) as the HCl salt. LCMS m/z=493.3 (M+1).

### Step 8. 1-[2-[4-[3-chloro-4-[2-hydroxy-1-(2-pyridyl)ethoxy]pyrazolo[1,5-a]pyridin-6-yl]-5-methyl-pyrazol-1-yl]-7-azaspiro[3.5]nonan-7-yl]prop-2-en-1-one

To a solution of 2-[6-[1-(7-azaspiro[3.5]nonan-2-yl)-5-methyl-pyrazol-4-yl]-3-chloro-pyrazolo[1,5-a]pyridin-4-yl]oxy-2-(2-pyridyl)ethanol HCl salt (240 mg, 0.45 mmol, 1.0 eq) and DIEA (292 mg, 2.27 mmol, 5. eq) in DCM (5 mL) was added prop-2-enoyl chloride (62 mg, 0.68 mmol, 1.5 eq). The resulting mixture was stirred at 0° C. for 2 h and was quenched with saturated NH₄Cl (5 mL) and extracted with DCM (10 mL×2). The combined organic layers were washed with brine (5 mL), dried over Na₂SO₄ and concentrated. The residue was purified by Prep-HPLC (Prep-C18, 5 μM Triart column, 20×150 mm, YMC-Actus; gradient elution of 44% MeCN in water to 66% MeCN in water over an 8 min period, where both solvents contain 0.05% NH₃·H₂O) to give a yellow solid (111 mg, 45% yield). LCMS m/z=547.4 (M+H); ¹H NMR (400 MHz, DMSO-d6) δ 8.59 (d, J=4.8 Hz, 1H), 8.23 (s, 1H), 8.04 (s, 1H), 7.82 (t, J=7.6 Hz, 1H), 7.63 (s, 1H), 7.56 (d, J=8.0 Hz, 1H), 7.32 (t, J=6.0 Hz, 1H), 6.87-6.76 (m, 1H), 6.58 (s, 1H), 6.08 (d, J=16.4 Hz, 1H), 5.66-5.59 (m, 2H), 5.11 (t, J=5.2 Hz, 1H), 4.95-4.87 (m, 1H), 3.95 (t, J=5.2 Hz, 2H), 3.53-3.45 (m, 4H), 2.39-2.28 (m, 4H), 2.17 (s, 3H), 1.68-1.57 (m, 4H).

### Step 9. Example 5 and Example 6

1-[2-[4-[3-chloro-4-[2-hydroxy-1-(2-pyridyl)ethoxy]pyrazolo[1,5-a]pyridin-6-yl]-5-methyl-pyrazol-1-yl]-7-azaspiro[3.5]nonan-7-yl]prop-2-en-1-one (101 mg) was separated by Prep-Chiral-HPLC (YMC CHIRAL ART Cellulose-SB column, DCXA-FLASH01; gradient elution of 30% IPA in n-Hexane to 30% IPA in n-Hexane over a 60 min period) to give Example 5 (rt=9.8 min; 36 mg, 35% yield) an off-white solid LCMS m/z=547.4 (M+H); ¹H NMR (400 MHz, DMSO-d6) δ=8.59 (d, J=4.8 Hz, 1H), 8.24 (s, 1H), 8.04 (s, 1H), 7.83 (t, J=7.6 Hz, 1H), 7.64 (s, 1H), 7.57 (d, J=8.0 Hz, 1H), 7.35-7.32 (m, 1H), 6.88-6.79 (m, 1H), 6.59 (s, 1H), 6.08 (d, J=16.4 Hz, 1H), 5.67-5.60 (m, 2H), 5.12 (brs, 1H), 4.96-4.88 (m, 1H), 3.95 (d, J=5.2 Hz, 2H), 3.54-3.45 (m, 4H), 2.39-2.28 (m, 4H), 2.17 (s, 3H), 1.68-1.57 (m, 4H). Example 6 (rt=12.3 min; 37 mg, 36% yield) as a white solid. LCMS m/z=547.4 (M+H); ¹H NMR (400 MHz, DMSO-d6) δ=8.59 (d, J=4.0 Hz, 1H), 8.23 (s, 1H), 8.04 (s, 1H), 7.83 (t, J=8.0 Hz, 1H), 7.63 (s, 1H), 7.57 (d, J=7.6 Hz, 1H), 7.35-7.32 (m, 1H), 6.83-6.80 (m, 1H), 6.58 (s, 1H), 6.08 (d, J=16.4 Hz, 1H), 5.66-5.60 (m, 2H), 4.94-4.89 (m, 1H), 3.94 (d, J=5.2 Hz, 2H), 3.53-3.45 (m, 4H), 2.38-2.27 (m, 4H), 2.17 (s, 3H), 1.68-1.57 (m, 4H).

### Examples 7 & 8

### (R)-6-(1-(7-acryloyl-7-azaspiro[3.5]nonan-2-yl)-5-methyl-1H-pyrazol-4-yl)-4-(2-hydroxy-1-(pyridin-2-yl)ethoxy)pyrazolo[1,5-a]pyridine-3-carbonitrile (Example 7) and (S)-6-(1-(7-acryloyl-7-azaspiro[3.5]nonan-2-yl)-5-methyl-1H-pyrazol-4-yl)-4-(2-hydroxy-1-(pyridin-2-yl)ethoxy)pyrazolo[1,5-a]pyridine-3-carbonitrile (Example 8)

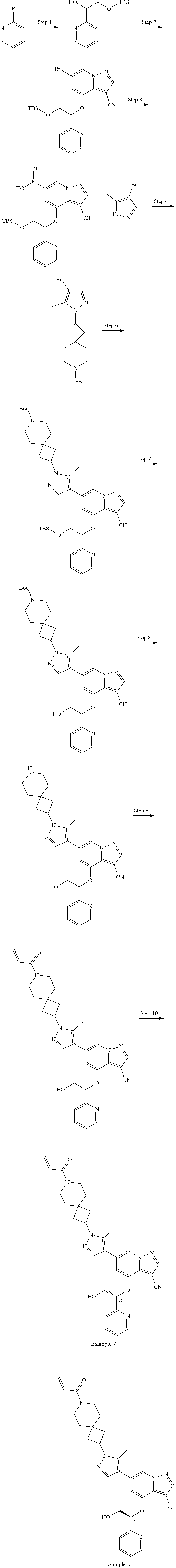

### Step 1. 2-[tert-butyl(dimethyl)silyl]oxy-1-(2-pyridyl)ethanol

To a solution of 2-bromopyridine (2.0 g, 12.7 mmol, 1.0 eq) in THF (20 mL) was added n-BuLi (7.3 mL, 18.99 mmol, 1.5 eq) at −78° C. under N₂ protection. The mixture was stirred for 0.5 h, then a solution 2-[tert-butyl(dimethyl)silyl]oxyacetaldehyde (3.31 g, 18.99 mmol, 1.5 eq) in THF (10 mL) was added dropwise to the reaction solution at −78° C. The solution was stirred for 0.5 h then was quenched with saturated aqueous NH₄Cl solution (30 mL) and extracted with ethyl acetate (30 mL×2). The combined organic layers were washed with brine (60 mL), dried over Na₂SO₄ and concentrated. The crude product was purified by silica gel column chromatography (Petroleum ether/ethyl acetate=10/1) to give a colorless oil (1.65 g, 51% yield). LCMS m/z=254.2 (M+1).

### Step 2. 6-bromo-4-[2-[tert-butyl(dimethyl)silyl]oxy-1-(2-pyridyl)ethoxy]pyrazolo[1,5-a]pyridine-3-carbonitrile

To a solution of 2-[tert-butyl(dimethyl)silyl]oxy-1-(2-pyridyl)ethanol (1.53 g, 6.05 mmol, 1.2 eq) and 6-bromo-4-hydroxy-pyrazolo[1,5-a]pyridine-3-carbonitrile (1.20 g, 5.04 mmol, 1.0 eq) in DCM (100 mL) were added PPh₃ (1.2 g, 10.08 mmol, 2.0 eq) and DIAD (1.53 mg, 7.56 mmol, 1.5 eq) at 0° C. The reaction solution was stirred for 12 h at rt. After the reaction was completed, the solution was poured into water (30 mL) and was extracted with DCM (30 mL×2). The combined organic layers were washed with brine (60 mL), dried over Na₂SO₄ and concentrated. The crude product was purified by silica gel column chromatography (Petroleum ether/ethyl acetate=5/1) to give a green solid (1.83 g, 77% yield). LCMS m/z=473.2 (M+1).

### Step 3. [4-[2-[tert-butyl(dimethyl)silyl]oxy-1-(2-pyridyl)ethoxy]-3-cyano-pyrazolo[1,5-a]pyridin-6-yl]boronic acid

To a solution of 6-bromo-4-[2-[tert-butyl(dimethyl)silyl]oxy-1-(2-pyridyl)ethoxy]-pyrazolo[1,5-a]pyridine-3-carbonitrile (1.00 g, 2.11 mmol, 1.0 eq) in dioxane (20 mL) was added KOAc (620 mg, 6.33 mmol, 3.0 eq), BPD (200 mg, 4.22 mmol, 2.0 eq) and Pd(PPh₃)₄ (230 mg, 0.21 mmol, 0.1 eq). The reaction mixture stirred for 1 h at 90° C. under nitrogen. After the reaction was completed, the reaction mixture was used directly to the next step. LCMS m/z=439.3 (M+1).

### Step 4. tert-butyl 2-(4-bromo-5-methyl-pyrazol-1-yl)-7-azaspiro[3.5]nonane-7-carboxylate

To a solution of 4-bromo-5-methyl-1H-pyrazole (1.50 g, 9.32 mmol, 1.0 eq) in DMA (30 mL) was added NaH (745 mg, 18.64 mmol, 2.0 eq). After stirring at rt for 1 h, tert-butyl 2-methylsulfonyloxy-7-azaspiro[3.5]nonane-7-carboxylate (2.98 g, 9.32 mmol, 1.0 eq) was added, and the resulting mixture stirred for 12 h at 70° C. The reaction mixture was quenched with aqueous NH₄Cl solution and was extracted with ethyl acetate (50 mL×2). The combined organic layers were washed with brine (50 mL), dried over Na₂SO₄, and concentrated in vacuum. The crude product was purified by silica gel column chromatography (Petroleum ether/ethyl acetate=10/1) to give a white solid (1.1 g, 31% yield). LCMS m/z=384.2 (M+1).

### Step 5. tert-butyl 2-[4-[4-[2-[tert-butyl(dimethyl)silyl]oxy-1-(2-pyridyl)ethoxy]-3-cyano-pyrazolo[1,5-a]pyridin-6-yl]-5-methyl-pyrazol-1-yl]-7-azaspiro[3.5]nonane-7-carboxylate

To a solution of tert-butyl 2-(4-bromo-5-methyl-pyrazol-1-yl)-7-azaspiro[3.5]nonane-7-carboxylate (85 mg, 0.22 mmol, 1.0 eq) in dioxane (3 mL) and H₂O (0.3 mL) was added [4-[2-[tert-butyl(dimethyl)silyl]oxy-1-(2-pyridyl)ethoxy]-3-cyano-pyrazolo[1,5-a]pyridin-6-yl]boronic acid (in dioxane), K₂CO₃ (61 mg, 0.44 mmol, 2.0 eq) and Pd(dppf)Cl₂ (19 mg, 0.02 mmol, 0.1 eq). The reaction mixture was stirred for 1.5 h at 90° C. under N₂ protection. After the reaction was completed, the mixture was poured into water (30 mL) and was extracted with ethyl acetate (30 mL×2). The combined organic layers were washed with brine (30 mL), dried over Na₂SO₄ and concentrated. The crude product was purified by silica gel column chromatography (petroleum ether/ethyl acetate=1/1) to give a white solid (64 mg, 42% yield). LCMS m/z=698.5 (M+1).

### Step 6. tert-butyl 2-[4-[3-cyano-4-[2-hydroxy-1-(2-pyridyl)ethoxy]pyrazolo[1,5-a]pyridin-6-yl]-5-methyl-pyrazol-1-yl]-7-azaspiro[3.5]nonane-7-carboxylate

To a solution of tert-butyl 2-[4-[4-[2-[tert-butyl(dimethyl)silyl]oxy-1-(2-pyridyl)-ethoxy]-3-cyano-pyrazolo[1,5-a]pyridin-6-yl]-5-methyl-pyrazol-1-yl]-7-azaspiro[3.5]nonane-7-carboxylate (280 mg, 0.40 mmol, 1.0 eq) in THF (8 mL) was added TBAF (157 mg, 0.60 mmol, 1.5 eq). The reaction mixture was stirred for 6 h at rt. The mixture was then diluted with ethyl acetate (50 mL) and washed with brine (20 mL×3). The organic layer was dried over Na₂SO₄ and concentrated. The crude product was purified by silica gel column chromatography (Petroleum ether/ethyl acetate=10/1) to give a yellow solid (210 mg, 90% yield). LCMS m/z=584.6 (M+1).

### Step 7. 6-[1-(7-azaspiro[3.5]nonan-2-yl)-5-methyl-pyrazol-4-yl]-4-[2-hydroxy-1-(2-pyridyl)ethoxy]pyrazolo[1,5-a]pyridine-3-carbonitrile HCl

To a solution of tert-butyl 2-[4-[3-cyano-4-[2-hydroxy-1-(2-pyridyl)ethoxy]pyrazolo-[1,5-a]pyridin-6-yl]-5-methyl-pyrazol-1-yl]-7-azaspiro[3.5]nonane-7-carboxylate (210 mg, 0.36 mmol, 1.0 eq) in DCM (5 mL) was added HCl/dioxane (5 mL; 4M). The reaction mixture was stirred for 3 h at rt, and then was concentrated in vacuum to give a yellow solid (210 mg) as the HCl salt. LCMS m/z=484.5 (M+1).

### Step 8. 4-[2-hydroxy-1-(2-pyridyl)ethoxy]-6-[5-methyl-1-(7-prop-2-enoyl-7-azaspiro-[3.5]nonan-2-yl)pyrazol-4-yl]pyrazolo[1,5-a]pyridine-3-carbonitrile

To a solution of −[1-(7-azaspiro[3.5]nonan-2-yl)-5-methyl-pyrazol-4-yl]-4-[2-hydroxy-1-(2-pyridyl)ethoxy]pyrazolo[1,5-a]pyridine-3-carbonitrile HCl salt (200 mg, 0.41 mmol, 1.0 eq) and DIEA (160 mg, 1.24 mmol, 3.0 eq) in DCM (6 mL) was added prop-2-enoyl chloride (26 mg, 0.29 mmol, 0.7 eq) at 0° C. The reaction mixture was stirred for 1 h at 0° C., then was diluted with DCM (30 mL), and washed with brine (10 mL×3). The organic layer was dried over Na₂SO₄ and concentrated. The residue was purified by Prep-HPLC (Prep-C18, 5 M Triart column, 19×150 mm, Phenyl OBD; gradient elution of 30% MeCN in water to 60% MeCN in water over a 9 min period, where both solvents contain 10 MMOL/L NH₄HCO₃-CAN) to give a light-yellow solid (112 mg, 50% yield). LCMS m/z=538.4 (M+¹); ¹H NMR (400 MHz, DMSO-d6) δ=538.4. 8.59 (s, 2H), 8.50 (s, 1H), 7.83 (t, J=7.6 Hz, 1H), 7.72 (s, 1H), 7.66 (d, J=8.0 Hz, 1H), 7.35-7.32 (m, 1H), 7.03 (s, 1H), 6.87-6.76 (m, 1H), 6.07 (d, J=16.4 Hz, 1H), 5.77 (t, J=5.6 Hz, 1H), 5.65 (d, J=10.4 Hz, 1H), 5.13 (t, J=5.6 Hz, 1H), 4.98-4.90 (m, 1H), 4.02-3.92 (m, 2H), 3.59-3.49 (m, 2H), 3.48-3.41 (m, 2H), 2.39-2.28 (m, 4H), 2.23 (s, 3H), 1.74-1.63 (m, 2H), 1.61-1.51 (m, 2H).

### Step 9. Example 7 and Example 8

4-[2-hydroxy-1-(2-pyridyl)ethoxy]-6-[5-methyl-1-(7-prop-2-enoyl-7-azaspiro[3.5]-nonan-2-yl)pyrazol-4-yl]pyrazolo[1,5-a]pyridine-3-carbonitrile (97 mg, 0.18 mmol, 1.0 eq) was separated by Prep-Chiral-HPLC (Celluiose-SB 100×4.6 mm 3 μm, DCXA-102; gradient elution of 1.0 mL-75% Hexane-25% EtOH-sb-20 min) to give example 7 (R)-6-(1-(7-acryloyl-7-azaspiro[3.5]nonan-2-yl)-5-methyl-1H-pyrazol-4-yl)-4-(2-hydroxy-1-(pyridin-2-yl)ethoxy)pyrazolo[1,5-a]pyridine-3-carbonitrile as a white solid (rt=9.8 min; 27 mg, 30% yield) LCMS m/z=538.3 (M+H); ¹H NMR (400 MHz, DMSO-d6) δ=8.60-8.58 (m, 2H), 8.50 (s, 1H), 7.83 (t, J=7.6 Hz, 1H), 7.73 (s, 1H), 7.66 (d, J=8.0 Hz, 1H), 7.36-7.33 (m, 1H), 7.03 (s, 1H), 6.87-6.76 (m, 1H), 6.08 (d, J=16.8 Hz, 1H), 5.77 (t, J=6.0 Hz, 1H), 5.65 (d, J=10.4 Hz, 1H), 5.12 (brs, 1H), 4.98-4.90 (m, 1H), 4.01-3.92 (m, 2H), 3.59-3.49 (m, 2H), 3.49-3.41 (m, 2H), 2.40-2.26 (m, 4H), 2.23 (s, 3H), 1.74-1.63 (m, 2H), 1.62-1.51 (m, 2H) and Example 8 (S)-6-(1-(7-acryloyl-7-azaspiro[3.5]nonan-2-yl)-5-methyl-1H-pyrazol-4-yl)-4-(2-hydroxy-1-(pyridin-2-yl)ethoxy)-pyrazolo[1,5-a]pyridine-3-carbonitrile as a white solid (rt=13.6 min; 22.1 mg, 23% yield) LCMS m/z=538.3 (M+H); ¹H NMR (400 MHz, DMSO-d6) δ=8.60-8.58 (m, 2H), 8.50 (s, 1H), 7.84 (t, J=7.6 Hz, 1H), 7.73 (s, 1H), 7.67 (d, J=7.6 Hz, 1H), 7.37-7.34 (m, 1H), 7.03 (s, 1H), 6.87-6.76 (m, 1H), 6.08 (d, J=16.8 Hz, 1H), 5.77 (t, J=6.0 Hz, 1H), 5.65 (d, J=10.4 Hz, 1H), 4.98-4.90 (m, 1H), 4.01-3.92 (m, 2H), 3.62-3.51 (m, 4H), 2.40-2.29 (m, 4H), 2.23 (s, 3H), 1.73-1.64 (m, 2H), 1.62-1.52 (m, 2H).

### Examples 9 & 10

### (S)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile (Example 9) and (R)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile (Example 10)

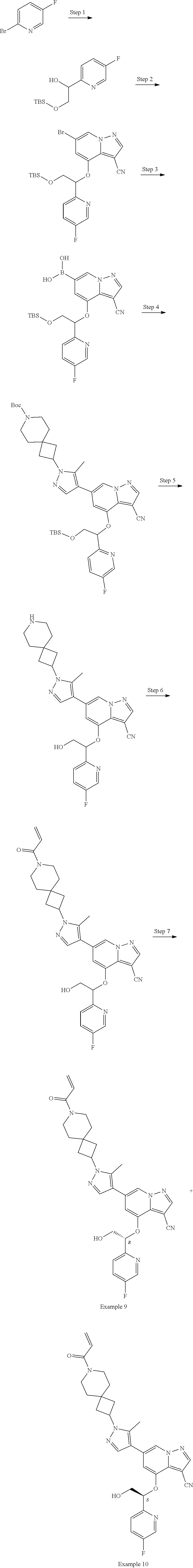

### Step 1. 2-[tert-butyl(dimethyl)silyl]oxy-1-(5-fluoro-2-pyridyl)ethanol

To a solution of 2-bromo-5-fluoro-pyridine (5.0 g, 30.0 mmol, 1.0 eq) in THF (40 mL) was added i-PrMgBr (2.0 M, 17.1 ml, 1.2 eq), dropwise in THF (10 mL) at 0° C. under nitrogen. After stirring for 30 min, 2-[tert-butyl(dimethyl)silyl]oxyacetaldehyde (6.3 g, 40.0 mmol, 1.2 eq) in THF (10 mL) was added, the resulting mixture was stirred at rt for 2 h. After the reaction was completed, the mixture was quenched with NH₄Cl solution (50 mL) and extracted with EtOAc (100 mL×2). The combined organic layers were dried over Na₂SO₄ and concentrated in vacuo. The crude product was purified by silica gel flash column chromatography (Petroleum Ether/EtOAc=3/1) to give a brown solid (5.4 g, 66% yield). LCMS m/z=272.3 (M+1).

### Step 2. 6-bromo-4-[2-[tert-butyl(dimethyl)silyl]oxy-1-(5-fluoro-2-pyridyl)ethoxy]-pyrazolo[1,5-a]pyridine-3-carbonitrile

To a solution of PPh₃ (3.2 g, 12.2 mmol, 1.5 eq) in THF (50 mL) was added DIAD (2.0 g, 9.7 mmol, 1.2 eq) at 0° C. Then 6-bromo-4-hydroxy-pyrazolo[1,5-a]pyridine-3-carbonitrile (2.9 g, 12.2 mmol, 1.5 eq) in THF (20 mL) was added to the reaction solution and stirred for 5 min under nitrogen protection. 2-[tert-Butyl(dimethyl)silyl]oxy-1-(5-fluoro-2-pyridyl)ethanol (2.2 g, 8.1 mmol, 1.0 eq) in THF (20 mL) was added at 0° C., and the resulting solution was stirred at rt for 3 h. The reaction mixture was then diluted with EtOAc (100 mL), and was washed with brine (100 mL). The organic layer was dried over Na₂SO₄ and concentrated in vacuo. The crude product was purified by silica gel flash column chromatography (petroleum ether/EtOAc=1/1) to give a yellow solid (2.5 g, 63% yield). LCMS m/z=491.3 (M+1).

### Step 3. [4-[2-[tert-butyl(dimethyl)silyl]oxy-1-(5-fluoro-2-pyridyl)ethoxy]-3-cyano-pyrazolo[1,5-a]pyridin-6-yl]boronic acid

A solution of 6-bromo-4-[2-[tert-butyl(dimethyl)silyl]oxy-1-(5-fluoro-2-pyridyl)-ethoxy]pyrazolo[1,5-a]pyridine-3-carbonitrile (1.0 g, 2.0 mmol, 1.0 eq) and BPD (1.0 g, 4.0 mmol, 2.0 eq) in dioxane (20 mL) was added KOAc (588 mg, 6.0 mmol, 3.0 eq) and Pd(dppf)Cl₂ (146 mg, 0.2 mmol, 0.1 eq) at rt. The reaction mixture was stirred at 90° C. for 3 h. After the reaction was completed, the solid was filtered out and the filtrate was concentrated in vacuo to give a crude brown solid (1.2 g) that was used in the next step. LCMS m/z=457.3 (M+1).

### Step 4. tert-butyl 2-[4-[4-[2-[tert-butyl(dimethyl)silyl]oxy-1-(5-fluoro-2-pyridyl) ethoxy]-3-cyano-pyrazolo[1,5-a]pyridin-6-yl]-5-methyl-pyrazol-1-yl]-7-azaspiro[3.5]nonane-7-carboxylate

To a solution of [4-[2-[tert-butyl(dimethyl)silyl]oxy-1-(5-fluoro-2-pyridyl)ethoxy]-3-cyano-pyrazolo[1,5-a]pyridin-6-yl]boronic acid (1.1 g, crude) and tert-butyl 2-(4-bromo-5-methyl-pyrazol-1-yl)-7-azaspiro[3.5]nonane-7-carboxylate (150 mg) in dioxane (10 mL) and H₂O (10 mL) was added Pd(dppf)Cl₂ (80 mg) and K₂CO₃ (161 mg). The reaction mixture was stirred at 90° C. for 30 min, and then was diluted with EtOAc (50 mL), which was washed with water (10 mL). The organic layer was dried over Na₂SO₄ and concentrated in vacuo. The crude product was purified by Prep-TLC (DCM/MeOH=20/1) to give a brown solid (230 mg, two steps, 18% yield). LCMS m/z=716.3 (M+1).

### Step 5. 6-[1-(7-azaspiro[3.5]nonan-2-yl)-5-methyl-pyrazol-4-yl]-4-[1-(5-fluoro-2-pyridyl)-2-hydroxy-ethoxy]pyrazolo[1,5-a]pyridine-3-carbonitrile

To a solution of tert-butyl 2-[4-[4-[2-[tert-butyl(dimethyl)silyl]oxy-1-(5-fluoro-2-pyridyl)ethoxy]-3-cyano-pyrazolo[1,5-a]pyridin-6-yl]-5-methyl-pyrazol-1-yl]-7-azaspiro[3.5]nonane-7-carboxylate (210 mg, 0.29 mmol, 1.0 eq) in DCM (4 mL) was added HCl/dioxane (2 mL, 4M). The reaction mixture was stirred at 30° C. for 1 h under N₂. After the reaction was completed, the reaction mixture was concentrated in vacuo, the pH value of the solution was adjusted to 7-8 with the saturated NaHCO₃ solution, and the solution was extracted with DCM (10 mL×3). The combined organic layers were dried over Na₂SO₄ and concentrated in vacuo to give a brown solid (160 mg). LCMS m/z=502.3 (M+1).

### Step 6. 6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile

To a solution of 6-[1-(7-azaspiro[3.5]nonan-2-yl)-5-methyl-pyrazol-4-yl]-4-[1-(5-fluoro-2-pyridyl)-2-hydroxy-ethoxy]pyrazolo[1,5-a]pyridine-3-carbonitrile (140 mg, crude) and DIEA (180 mg) in DCM (3 mL) was added prop-2-enoyl chloride (30 mg) in DCM (1 mL) at 0° C. under N₂ protection. The reaction mixture was stirred at 30° C. for 2 h, then was diluted with DCM (10 mL), washed with H₂O (5 mL), dried over Na₂SO₄ and concentrated. The crude product was purified by Prep-HPLC (Prep-C18, 5 μM Triart column, 20× 150 mm, YMC-Actus; gradient elution of 40% MeCN in water to 60% MeCN in water over a 11 min period, where both solvents contain 0.05% NH₃·H₂O) to give a white solid (80 mg, two steps, 55% yield). LCMS m/z=556.3 (M+1); ¹H NMR (400 MHz, DMSO-d6) δ 8.60-8.59 (m, 2H), 8.51 (s, 1H), 7.81-7.73 (m, 3H), 7.06 (s, 1H), 6.85-6.79 (m, 1H), 6.08 (d, J=16.0 Hz, 1H), 5.82 (t, J=6.0 Hz, 1H), 5.65 (d, J=10.4 Hz, 1H), 5.12-4.99 (m, 1H), 4.99-4.91 (m, 1H), 3.99-3.92 (m, 2H), 3.55-3.54 (m, 2H), 3.46-3.45 (m, 2H), 2.38-2.32 (m, 4H), 2.25 (s, 3H), 1.69-1.68 (m, 2H), 1.58-1.56 (m, 2H).

### Step 7. Example 9 and Example 10

6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile (70 mg) was separated by Prep-Chiral-HPLC (YMC CHIRAL ART Cellulose-SB column, DCXA-FLASH01; gradient elution of 45% IPA in n-Hexane to 45% IPA in n-Hexane over a 29 min period) to give(S)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile as a white solid (rt=3.5 min; 22 mg, 31% yield); LCMS m/z=556.3 (M+H); ¹H NMR (400 MHz, DMSO-d6) δ=8.60-8.59 (m, 2H), 8.51 (s, 1H), 7.81-7.73 (m, 3H), 7.06 (s, 1H), 6.85-6.79 (m, 1H), 6.08 (d, J=16.0 Hz, 1H), 5.82 (t, J=5.6 Hz, 1H), 5.65 (d, J=10.0 Hz, 1H), 5.11 (t, J=5.6 Hz, 1H), 4.97-4.93 (m, 1H), 4.00-3.93 (m, 2H), 3.55-3.53 (m, 2H), 3.47-3.46 (m, 2H), 2.41-2.30 (m, 4H), 2.25 (s, 3H), 1.70-1.69 (m, 2H), 1.59-1.57 (m, 2H) and Example 10 (R)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile as a white solid (rt=8.1 min; 21 mg, 30% yield); LCMS m/z=556.4 (M+H); ¹H NMR (400 MHz, DMSO-d6) δ=8.60-8.59 (m, 2H), 8.51 (s, 1H), 7.81-7.73 (m, 3H), 7.06 (s, 1H), 6.87-6.76 (m, 1H), 6.08 (d, J=16.8 Hz, 1H), 5.81 (t, J=5.6 Hz, 1H), 5.65 (d, J=10.4 Hz, 1H), 5.11 (t, J=5.6 Hz, 1H), 5.00-4.91 (m, 1H), 4.01-3.91 (m, 2H), 3.54-3.53 (m, 2H), 3.47-3.44 (m, 2H), 2.41-2.28 (m, 4H), 2.25 (s, 3H), 1.70-1.69 (m, 2H), 1.59-1.57 (m, 2H).

### Example 11. 6-(1-(7-acryloyl-7-azaspiro[3.5]nonan-2-yl)-5-methyl-1H-pyrazol-4-yl)-4-(2-hydroxy-1-(1-methyl-1H-pyrazol-5-yl)ethoxy)pyrazolo[1,5-a]pyridine-3-carbonitrile

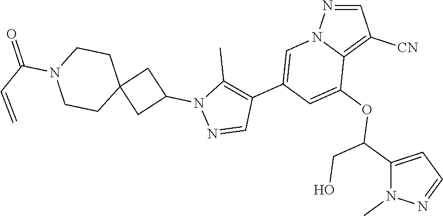 [SMILES: C=CC(=O)N1CCC2(CC1)CC(n1ncc(-c3cc(OC(CO)c4ccnn4C)c4c(C#N)cnn4c3)c1C)C2]

Example 11 was made by methods analogous to those described above.

LCMS m/z=613.2 (M+H); ¹H NMR (400 MHz, DMSO-d6) δ=8.57 (s, 1H), 8.52 (d, J=0.8 Hz, 1H), 7.77 (s, 1H), 7.36 (d, J=2.0 Hz, 1H), 7.21 (s, 1H), 6.87-6.76 (m, 1H), 6.43 (d, J=2.0 Hz, 1H), 6.10-6.02 (m, 2H), 5.65 (d, J=10.8 Hz, 1H), 5.34 (t, J=6.0 Hz, 1H), 5.01-4.93 (m, 1H), 4.08-3.98 (m, 1H), 3.91 (s, 3H), 3.88-3.83 (m, 1H), 3.58-3.51 (m, 2H), 3.50-3.42 (m, 2H), 2.41-2.33 (m, 4H), 2.23 (s, 3H), 1.74-1.64 (m, 2H), 1.63-1.54 (m, 2H).

### Examples 12 & 13

### (R)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile (Example 12) and (S)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile (Example 13)

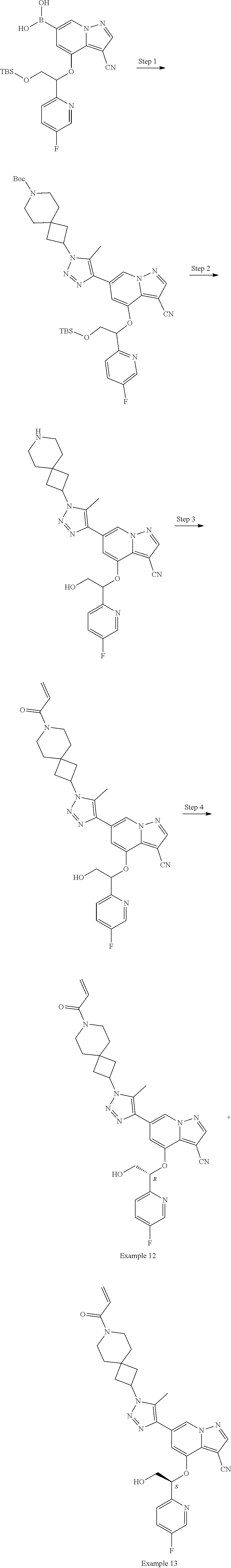

### Step 1. tert-butyl 2-[4-[4-[2-[tert-butyl(dimethyl)silyl]oxy-1-(5-fluoro-2-pyridyl) ethoxy]-3-cyano-pyrazolo[1,5-a]pyridin-6-yl]-5-methyl-triazol-1-yl]-7-azaspiro[3.5]nonane-7-carboxylate

To a solution of 4-[2-[tert-butyl(dimethyl)silyl]oxy-1-(5-fluoro-2-pyridyl)ethoxy]-6-(4,4,5,5-tetramethyl-1,3,2-dioxaborolan-2-yl)pyrazolo[1,5-a]pyridine-3-carbonitrile (300 mg) and tert-butyl 2-(4-bromo-5-methyl-triazol-1-yl)-7-azaspiro[3.5]nonane-7-carboxylate (146 mg, 0.39 mmol) in dioxane (5 mL) and H₂O (0.5 mL) was added Pd(dppf)Cl₂ (21 mg, 0.033 mmol) and Cs₂CO₃ (318 mg, 0.98 mmol). The reaction mixture was stirred at 90° C. for 5 h under N₂ protection. After the reaction was completed, the mixture was diluted with EtOAc (20 mL) and washed with water (10 mL). The organic layer was dried over Na₂SO₄ and concentrated in vacuo. The crude product was purified by Prep-TLC (DCM/MeOH=20/1) to give a brown solid (210 mg). LCMS m/z=717.3 (M+1).

### Step 2. 6-[1-(7-azaspiro[3.5]nonan-2-yl)-5-methyl-triazol-4-yl]-4-[1-(5-fluoro-2-pyridyl)-2-hydroxy-ethoxy]pyrazolo[1,5-a]pyridine-3-carbonitrile

To a solution of tert-butyl 2-[4-[4-[2-[tert-butyl(dimethyl)silyl]oxy-1-(5-fluoro-2-pyridyl)ethoxy]-3-cyano-pyrazolo[1,5-a]pyridin-6-yl]-5-methyl-triazol-1-yl]-7-azaspiro[3.5]nonane-7-carboxylate (190 mg, 0.34 mmol, 1.0 eq) in DCM (5 mL) was added HCl/dioxane (2 mL, 4M). The reaction mixture was stirred at rt for 5 h and concentrated in vacuo. The pH of the solution was adjusted to 7-8 with the saturated NaHCO₃ solution and extracted with DCM (10 mL×3). The combined organic layers were dried over Na₂SO₄ and concentrated in vacuo to a brown solid (130 mg). LCMS m/z=503.3 (M+1).

### Step 3. 4-[1-(5-fluoro-2-pyridyl)-2-hydroxy-ethoxy]-6-[5-methyl-1-(7-prop-2-enoyl-7-azaspiro[3.5]nonan-2-yl)triazol-4-yl]pyrazolo[1,5-a]pyridine-3-carbonitrile

To a solution of 6-[1-(7-azaspiro[3.5]nonan-2-yl)-5-methyl-triazol-4-yl]-4-[1-(5-fluoro-2-pyridyl)-2-hydroxy-ethoxy]pyrazolo[1,5-a]pyridine-3-carbonitrile (120 mg, crude) and TEA (120 mg, 1.19 mmol) in DCM (3 mL) was added prop-2-enoyl chloride (26 mg, 0.29 mmol) in DCM (1 mL) at 0° C. The reaction mixture was stirred at rt for 2 h, diluted with DCM (10 mL), washed with H₂O (5 mL), and dried over Na₂SO₄ and concentrated. The crude product was purified by Prep-HPLC (Prep-C18, 5 μM Triart column, 20×150 mm, YMC-Actus; gradient elution of 40% MeCN in water to 60% MeCN in water over a 11 min period, where both solvents contain 0.05% NH₃·H₂O) to give a white solid (100 mg, two steps, 48% yield). LCMS m/z=557.5 (M+1).

### Step 4. Example 12 and Example 13

(R)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile and (S)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile. 4-[1-(5-fluoro-2-pyridyl)-2-hydroxy-ethoxy]-6-[5-methyl-1-(7-prop-2-enoyl-7-azaspiro[3.5]nonan-2-yl)triazol-4-yl]pyrazolo[1,5-a]pyridine-3-carbonitrile (100 mg) was separation by Prep-Chiral-HPLC (YMC CHIRAL ART Cellulose-SB column, DCXA-FLASH01; gradient elution of 45% IPA in n-Hexane to 45% IPA in n-Hexane over a 29 min period) to give (R)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile as a white solid (rt=6.5 min; 35.1 mg, 35% yield); LCMS m/z=557.5 (M+H); ¹H NMR (400 MHz, DMSO-d6) δ=8.68 (s, 1H), 8.66 (s, 1H), 8.61 (d, J=2.8 Hz, 1H), 7.80-7.77 (m, 1H), 7.74-7.72 (m, 1H), 7.30 (s, 1H), 6.86-6.78 (m, 1H), 6.09 (d, J=17.2 Hz, 1H), 5.76 (t, J=5.6 Hz, 1H), 5.66 (d, J=10.0 Hz, 1H), 5.12-5.06 (m, 1H), 3.99-3.96 (m, 2H), 3.57-3.56 (m, 2H), 3.47-3.46 (m, 2H), 2.56-2.51 (m, 2H), 2.45-2.38 (m, 5H), 1.72-1.70 (m, 2H), 1.62-1.59 (m, 2H) and (S)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile as a white solid (rt=7.7 min; 37 mg, 37% yield) LCMS m/z=557.4 (M+H); ¹H NMR (400 MHz, DMSO-d6) δ=8.67 (s, 1H), 8.65 (s, 1H), 8.60 (s, 1H), 7.81-7.77 (m, 1H), 7.74-7.70 (m, 1H), 7.29 (s, 1H), 6.88-6.77 (m, 1H), 6.09 (d, J=17.2 Hz, 1H), 5.76 (t, J=4.0 Hz, 1H), 5.66 (d, J=10.8 Hz, 1H), 5.17-5.08 (m, 2H), 3.97-3.93 (m, 2H), 3.56-3.52 (m, 2H), 3.47-3.43 (m, 2H), 2.67-2.58 (m, 2H), 2.44-2.38 (m, 5H), 1.72-1.68 (m, 2H), 1.60-1.58 (m, 2H).

### Example 14. 6-(1-(7-acryloyl-7-azaspiro[3.5]nonan-2-yl)-5-methyl-1H-pyrazol-4-yl)-4-(2-hydroxy-1-(1-methyl-1H-pyrazol-3-yl)ethoxy)pyrazolo[1,5-a]pyridine-3-carbonitrile

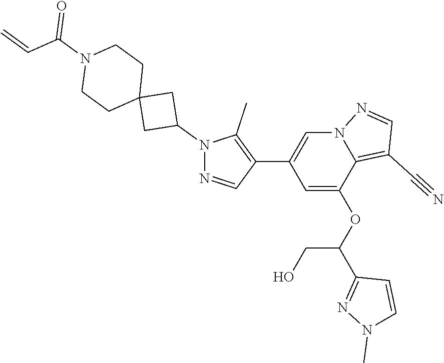 [SMILES: C=CC(=O)N1CCC2(CC1)CC(n1ncc(-c3cc(OC(CO)c4ccn(C)n4)c4c(C#N)cnn4c3)c1C)C2]

Example 14 was made by methods analogous to those described above.

LCMS m/z=541.6 (M+H); ¹H NMR (400 MHz, DMSO-d6) δ=8.55 (s, 1H), 8.49 (s, 1H), 7.77 (s, 1H), 7.64 (d, J=2.0 Hz, 1H), 7.19 (s, 1H), 6.85-6.80 (m, 1H), 6.35 (d, J=2.0 Hz, 1H), 6.10 (d, J=17.2 Hz, 1H), 5.68-5.64 (m, 2H), 5.08 (t, J=5.6 Hz, 1H), 5.02-4.94 (m, 1H), 4.00-3.94 (m, 1H), 3.85-3.81 (m, 1H), 3.80 (s, 3H), 3.57-3.44 (m, 4H), 2.42-2.34 (m, 7H), 1.73-1.57 (m, 4H).

### Example 15. 6-(1-(7-acryloyl-7-azaspiro[3.5]nonan-2-yl)-5-methyl-1H-pyrazol-4-yl)-4-(2-hydroxy-1-(isoquinolin-4-yl)ethoxy)pyrazolo[1,5-a]pyridine-3-carbonitrile

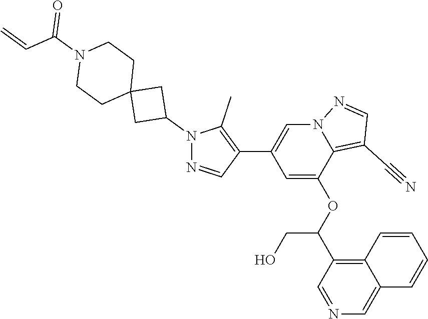 [SMILES: C=CC(=O)N1CCC2(CC1)CC(n1ncc(-c3cc(OC(CO)c4cncc5ccccc45)c4c(C#N)cnn4c3)c1C)C2]

Example 15 was made by methods analogous to those described above.

LCMS m/z=588.6 (M+H); ¹H NMR (400 MHz, DMSO-d6) δ=9.30 (s, 1H), 8.84 (s, 1H), 8.61-8.59 (m, 2H), 8.47 (s, 1H), 8.18 (d, J=8.0 Hz, 1H), 7.89-7.85 (m, 1H), 7.73 (t, J=7.2 Hz, 1H), 7.68 (s, 1H), 7.19 (s, 1H), 6.88-6.76 (m, 1H), 6.48 (t, J=5.6 Hz, 1H), 6.08 (d, J=16.8 Hz, 1H), 5.66 (d, J=10.4 Hz, 1H), 5.32 (brs, 1H), 4.95-4.86 (m, 1H), 4.18-4.15 (m, 1H), 4.03-3.98 (m, 1H), 3.56-3.51 (m, 2H), 3.46-3.43 (m, 2H), 2.38-2.26 (m, 4H), 2.09 (s, 3H), 1.71-1.66 (m, 2H), 1.60-1.54 (m, 2H).

### Example 16. 4-(2-hydroxy-1-(pyridin-2-yl)ethoxy)-6-(5-methyl-1-(1-(1-propionylazetidine-3-carbonyl)piperidin-4-yl)-1H-1,2,3-triazol-4-yl)pyrazolo[1,5-a]pyridine-3-carbonitrile

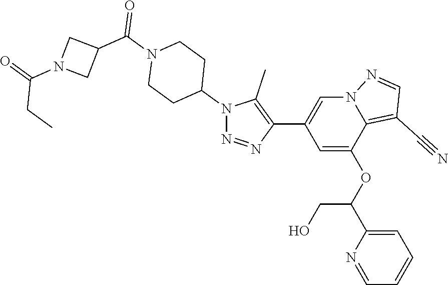 [SMILES: CCC(=O)N1CC(C(=O)N2CCC(n3nnc(-c4cc(OC(CO)c5ccccn5)c5c(C#N)cnn5c4)c3C)CC2)C1]

Example 16 was made by methods analogous to those described above.

LCMS m/z=584.3 (M+H); ¹H NMR (400 MHz, DMSO-d6) δ=8.68 (s, 1H), 8.66 (s, 1H), 8.59 (d, J=4.4 Hz, 1H), 7.86-7.81 (m, 1H), 7.63 (d, J=8.0 Hz, 1H), 7.36-7.33 (m, 1H), 7.28 (s, 1H), 5.72 (t, J=6.0 Hz, 1H), 5.17 (t, J=5.6 Hz, 1H), 4.74-4.68 (m, 1H), 4.53-4.49 (m, 1H), 4.30-4.17 (m, 2H), 4.05-3.87 (m, 4H), 3.81-3.71 (m, 2H), 3.25-3.20 (m, 1H), 2.88 (t, J=11.6 Hz, 1H), 2.44 (s, 3H), 2.07-2.00 (m, 5H), 1.93-1.86 (m, 1H), 0.95 (t, J=7.6 Hz, 3H).

### Examples 17 & 18

### (R)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile (Example 17) and (S)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile (Example 18)

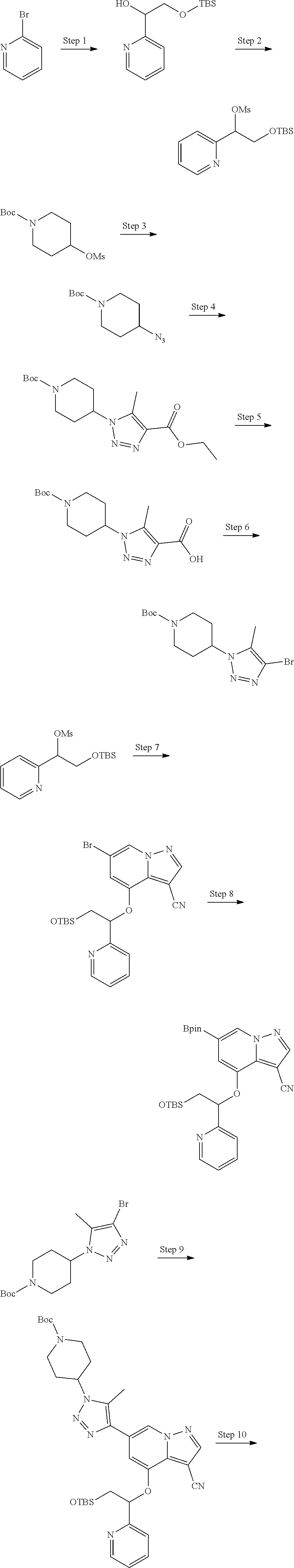

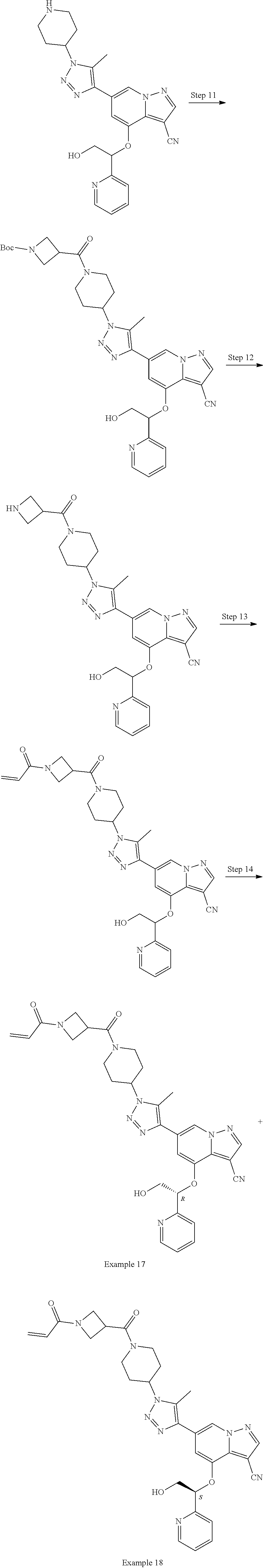

### Step 1. 2-[tert-butyl(dimethyl)silyl]oxy-1-(2-pyridyl)ethanol

To a solution of 2-bromopyridine (2.0 g, 12.7 mmol, 1.0 eq) in THF (20 mL) was added n-BuLi (7.3 mL, 18.99 mmol, 1.5 eq) at −78° C. under N₂ protection. The solution was stirred for 0.5 h, then 2-[tert-butyl(dimethyl)silyl]oxyacetaldehyde (3.31 g, 18.99 mmol, 1.5 eq) in THF (10 mL) was added dropwise. The reaction solution was stirred at −78° C. for 0.5 h, and then was quenched with NH₄Cl (30 mL) and extracted with ethyl acetate (50 mL×2). The combined organic layers were washed with brine (30 mL×2), dried over Na₂SO₄, and concentrated in vacuum. The crude was purified by silica gel column chromatography (petroleum ether/ethyl acetate=5/1) to give a colorless oil (1.65 g, 51% yield). LCMS m/z=254.2 (M+1).

### Step 2. [2-[tert-butyl(dimethyl)silyl]oxy-1-(2-pyridyl)ethyl]methanesulfonate

To a solution of 2-[tert-butyl(dimethyl)silyl]oxy-1-(2-pyridyl)ethanol (1.5 g, 5.9 mmol, 1.0 eq) in DCM (20 mL) was added TEA (1.79 mg, 17.7 mmol, 3.0 eq) at 0° C. under N₂ protection. Then, MsCl (1.0 g, 8.86 mmol, 1.5 eq) in DCM (5 mL) was added dropwise at 0° C. The solution was stirred at rt for 2 h, then was quenched with H₂O (30 mL) and extracted with DCM (30 mL×2). The combined organic layers were washed with brine (20 mL), dried over Na₂SO₄, and concentrated in vacuum. The crude was purified by silica gel column chromatography (petroleum ether/ethyl acetate=10/1) to give a yellow solid (1.5 g, 77% yield). LCMS m/z=332.1 (M+1).

### Step 3. tert-butyl 4-azidopiperidine-1-carboxylate

To a solution of tert-butyl 4-methylsulfonyloxypiperidine-1-carboxylate (19.0 g, 68.01 mmol, 1.0 eq) in DMF (200 mL) was added NaN₃ (13.26 g, 204.04 mmol, 3.0 eq) at 0° C. The reaction mixture was stirred at 100° C. for 2 h, then cooled to rt, ice-water (1 L) was added to the reaction mixture and extracted with EtOAc (300 mL×3). The combined organic layers were dried over Na₂SO₄ and concentrated in vacuo to give a yellow oil (14 g, 91% yield). LCMS m/z=227.4 (M+1).

### Step 4. tert-butyl 4-(4-ethoxycarbonyl-5-methyl-triazol-1-yl) piperidine-1-carboxylate

To a solution of tert-butyl 4-azidopiperidine-1-carboxylate (14.0 g, 61.87 mmol, 1.0 eq) and ethyl 3-oxobutanoate (12.1 g, 92.8 mmol, 1.5 eq) in DMSO (140 mL) was added K₂CO₃ (25.62 g, 185.68 mmol, 3.0 eq). The reaction mixture was stirred at 80° C. for 16 h. After the reaction was completed, the mixture was diluted with EtOAc (1 L) and washed with brine (200 mL×5). The organic layer was dried over Na₂SO₄ and concentrated. The crude product was purified by silica gel flash column chromatography (petroleum ether/EtOAc=1/1) to give a yellow oil (17.5 g, 84% yield). LCMS m/z=339.5 (M+1).

### Step 5. 1-(1-tert-butoxycarbonyl-4-piperidyl)-5-methyl-triazole-4-carboxylic acid

To a solution of tert-butyl 4-(4-ethoxycarbonyl-5-methyl-triazol-1-yl) piperidine-1-carboxylate (17.5 g, 51.71 mmol, 1.0 eq) in MeOH (70 mL) and H₂O (70 mL) was added KOH (5.8 g, 103.4 mmol, 2.0 eq). The reaction mixture was stirred at 50° C. for 2 h. After the reaction was completed, the pH of the mixture was adjusted to 5 by addition of hydrochloric acid solution (1 M) and extracted with DCM (300 mL×3). The combined organic layers were dried over Na₂SO₄ and concentrated in vacuo to give a yellow solid (14.9 g, 93% yield). LCMS m/z=311.2 (M+1).

### Step 6. tert-butyl 4-(4-bromo-5-methyl-triazol-1-yl) piperidine-1-carboxylate

To a solution of 1-(1-tert-butoxycarbonyl-4-piperidyl)-5-methyl-triazole-4-carboxylic acid (14.0 g, 45.1 mmol, 1.0 eq) and KOH (3.04 g, 54.1 mmol, 1.2 eq) in H₂O (200 mL) was added Br₂ (8.66 g, 54.1 mmol, 1.2 eq) at 0° C. The mixture was stirred at rt for 4 h. After the reaction was completed, the mixture was quenched with the saturated Na₂SO₃ solution, and extracted with EtOAc (300 mL×3). The combined organic layers were concentrated, and the crude product was purified by silica gel chromatography (petroleum ether/EtOAc=2/1) to give a yellow solid (12.3 g, 79% yield). LCMS m/z=345.2 (M+1).

### Step 7. 6-bromo-4-[2-[tert-butyl(dimethyl)silyl]oxy-1-(2-pyridyl)ethoxy]pyrazolo[1,5-a]pyridine-3-carbonitrile

To a solution of [2-[tert-butyl(dimethyl)silyl]oxy-1-(2-pyridyl)ethyl]methanesulfonate (9.3 g, 28.1 mmol, 1.0 eq) and 6-bromo-4-hydroxy-pyrazolo[1,5-a]pyridine-3-carbonitrile (10.0 g, 42.2 mmol, 1.5 eq) in DMF (150 mL) was added K₂CO₃ (11.6 g, 84.3 mmol, 3.0 eq) at rt. The reaction mixture was stirred at 70° C. for 12 h under N₂ protection. After the reaction was completed, the mixture was diluted with EtOAc (300 mL) and washed with brine (100 mL). The organic layer was dried over Na₂SO₄ and concentrated in vacuo. The crude product was purified by silica gel flash column chromatography (petroleum ether/EtOAc=2/1) to give a brown solid (12.1 g, 91% yield). LCMS m/z=473.3 (M+1).

### Step 8. 4-[2-[tert-butyl(dimethyl)silyl]oxy-1-(2-pyridyl)ethoxy]-6-(4,4,5,5-tetramethyl-1,3,2-dioxaborolan-2-yl)pyrazolo[1,5-a]pyridine-3-carbonitrile

To a solution of 6-bromo-4-[2-[tert-butyl(dimethyl)silyl]oxy-1-(2-pyridyl)ethoxy]-pyrazolo[1,5-a]pyridine-3-carbonitrile (10.0 g, 21.2 mmol, 1.0 eq) and BPD (10.8 g, 42.4 mmol, 2.0 eq) in dioxane (150 mL) was added KOAc (6.2 g, 63.6 mmol, 3.0 eq) and Pd(PPh₃)₄ (1.2 g, 1.1 mmol, 0.05 eq). The reaction mixture was stirred at 90° C. for 1.5 h under N₂ then was concentrated to give a brown solid (28 g). LCMS m/z=439.3 (M+1).

### Step 9. tert-butyl 4-[4-[4-[2-[tert-butyl(dimethyl)silyl]oxy-1-(2-pyridyl)ethoxy]-3-cyano-pyrazolo[1,5-a]pyridin-6-yl]-5-methyl-triazol-1-yl]piperidine-1-carboxylate

To a solution of 4-[2-[tert-butyl(dimethyl)silyl]oxy-1-(2-pyridyl)ethoxy]-6-(4,4,5,5-tetramethyl-1,3,2-dioxaborolan-2-yl)pyrazolo[1,5-a]pyridine-3-carbonitrile (28.0 g, crude) and tert-butyl 4-(4-bromo-5-methyl-triazol-1-yl) piperidine-1-carboxylate (8.5 g) in dioxane (200 mL), H₂O (20 mL) was added Pd(dtbpf)Cl₂ (1.0 g) and K₂CO₃ (9.5 g). The reaction mixture was stirred at 90° C. for 2 h under N₂, and then was concentrated in vacuo. The residue was diluted with EtOAc (200 mL), washed with brine (100 mL), dried over Na₂SO₄ and concentrated in vacuo. The crude product was purified by silica gel flash column chromatography (petroleum ether/EtOAc=1/1) to give a yellow solid (12.0 g, two steps, 86% yield). LCMS m/z=659.3 (M+1).

### Step 10. 4-[2-hydroxy-1-(2-pyridyl)ethoxy]-6-[5-methyl-1-(4-piperidyl)triazol-4-yl]pyrazolo[1,5-a]pyridine-3-carbonitrile HCl

To a solution of tert-butyl 4-[4-[4-[2-[tert-butyl(dimethyl)silyl]oxy-1-(2-pyridyl) ethoxy]-3-cyano-pyrazolo[1,5-a]pyridin-6-yl]-5-methyl-triazol-1-yl]piperidine-1-carboxylate (11.2 g, 17.0 mmol, 1.0 eq) in DCM (100 mL) was added HCl/dioxane (40 mL, 4M). The reaction mixture was stirred at rt for 1 h under N₂ protection and then was concentrated to give a brown solid (8.7 g) as the HCl salt. LCMS m/z=445.3 (M+1).

### Step 11. tert-butyl 3-[4-[4-[3-cyano-4-[2-hydroxy-1-(2-pyridyl)ethoxy]pyrazolo[1,5-a]pyridin-6-yl]-5-methyl-triazol-1-yl]piperidine-1-carbonyl]azetidine-1-carboxylate

To a solution of 1-tert-butoxycarbonylazetidine-3-carboxylic acid (60.3 mg, 0.3 mmol, 1.5 eq), HATU (418 mg, 1.1 mmol, 1.0 eq) and DIEA (710 mg, 5.5 mmol, 5.0 eq) in DCM (15 mL) was added 4-[2-hydroxy-1-(2-pyridyl)ethoxy]-6-[5-methyl-1-(4-piperidyl)triazol-4-yl]pyrazolo[1,5-a]pyridine-3-carbonitrile HCl (500 mg, 1.1 mmol, 1.0 eq) in DCM (5 mL) at 0° C. The reaction mixture was stirred at rt for 1 h under N₂, then was diluted with DCM (50 mL), washed with brine (30 mL) and concentrated. The crude product was purified by silica gel flash column chromatography (petroleum ether/EtOAc=1/1) to give a yellow solid (380 mg, 54% yield). LCMS m/z=628.3 (M+1).

### Step 12. 6-[1-[1-(azetidine-3-carbonyl)-4-piperidyl]-5-methyl-triazol-4-yl]-4-[2-hydroxy-1-(2-pyridyl)ethoxy]pyrazolo[1,5-a]pyridine-3-carbonitrile

To a solution of tert-butyl 3-[4-[4-[3-cyano-4-[2-hydroxy-1-(2-pyridyl)ethoxy]pyrazolo[1,5-a]pyridin-6-yl]-5-methyl-triazol-1-yl]piperidine-1-carbonyl]azetidine-1-carboxylate (320 mg, 0.51 mmol, 1.0 eq) in DCM (4 mL) was added HCl/dioxane (2 mL, 4M). The reaction mixture was stirred at rt for 1 h under N₂ and then was concentrated to give a brown solid (400 mg) as the HCl salt. LCMS m/z=528.3 (M+1).

### Step 13. 4-[2-hydroxy-1-(2-pyridyl)ethoxy]-6-[5-methyl-1-[1-(1-prop-2-enoylazetidine-3-carbonyl)-4-piperidyl]triazol-4-yl]pyrazolo[1,5-a]pyridine-3-carbonitrile

To a solution of 6-[1-[1-(azetidine-3-carbonyl)-4-piperidyl]-5-methyl-triazol-4-yl]-4-[2-hydroxy-1-(2-pyridyl)ethoxy]pyrazolo[1,5-a]pyridine-3-carbonitrile (270 mg, crude) and K₂CO₃ (120 mg) in DCM (3 mL) was added prop-2-enoyl chloride (25 mg) in DCM (1 mL) at 0° C. under N₂. The reaction mixture was stirred at rt for 2 h, then was diluted with DCM (10 mL), washed with H₂O (5 mL), dried over Na₂SO₄ and was concentrated. The crude product was purified by Prep-TLC (DCM/MeOH=15/1) to give a white solid (80 mg, two steps, 23% yield). LCMS m/z=582.3 (M+1).

### Step 14. Example 17 and Example 18

(R)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile and (S)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile. 4-[2-hydroxy-1-(2-pyridyl)ethoxy]-6-[5-methyl-1-[1-(1-prop-2-enoylazetidine-3-carbonyl)-4-piperidyl]triazol-4-yl]pyrazolo[1,5-a]pyridine-3-carbonitrile (80 mg) was separated by Prep-Chiral-HPLC (YMC CHIRAL ART Cellulose-SB column, DCXA-FLASH01; gradient elution of 45% IPA in n-Hexane to 45% IPA in n-Hexane over a 29 min period) to give (R)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile as a white solid (peak 1, 23.5 mg, 29% yield); LCMS m/z=582.5 (M+H); ¹H NMR (400 MHz, DMSO-d6) δ=8.68 (s, 1H), 8.66 (s, 1H), 8.59 (d, J=4.4 Hz, 1H), 7.85-7.82 (m, 1H), 7.63 (d, J=7.6 Hz, 1H), 7.37-7.34 (m, 1H), 7.29 (s, 1H), 6.38-6.28 (m, 1H), 6.11 (d, J=16.8 Hz, 1H), 5.73 (t, J=6.0 Hz, 1H), 5.68 (d, J=10.4 Hz, 1H), 5.16 (brs, 1H), 4.71-4.69 (m, 1H), 4.51 (d, J=13.6 Hz, 1H), 4.43-4.29 (m, 2H), 4.13 (t, J=9.2 Hz, 1H), 4.07-3.93 (m, 3H), 3.86-3.79 (m, 1H), 3.76-3.72 (m, 1H), 3.24-3.20 (m, 1H), 2.88 (t, J=12.4 Hz, 1H), 2.45 (s, 3H), 2.06-2.01 (m, 3H), 1.94-1.85 (m, 1H); and (S)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile as a white solid (peak 2; 24 mg, 30% yield) LCMS m/z=582.6 (M+H); ¹H NMR (400 MHz, DMSO-d6) δ=8.68 (s, 1H), 8.66 (s, 1H), 8.59 (d, J=7.6 Hz, 1H), 7.86-7.81 (m, 1H), 7.63 (d, J=8.0 Hz, 1H), 7.36-7.33 (m, 1H), 7.29 (s, 1H), 6.38-6.28 (m, 1H), 6.10 (dd, J=17.2 Hz, 2.0 Hz, 1H), 5.72 (t, J=6.0 Hz, 1H), 5.67 (d, J=2.8 Hz, 1H), 5.23 (brs, 1H), 4.72-4.69 (m, 1H), 4.51 (d, J=13.2 Hz, 1H), 4.40-4.29 (m, 2H), 4.13 (t, J=9.2 Hz, 1H), 4.07-3.96 (m, 3H), 3.86-3.72 (m, 2H), 3.26-3.20 (m, 1H), 2.88 (t, J=12.0 Hz, 1H), 2.45 (s, 3H), 2.06-2.01 (m, 3H), 1.91-1.87 (m, 1H).

### Examples 19 & 20

### (R)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile (Example 19) and (S)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile (Example 20)

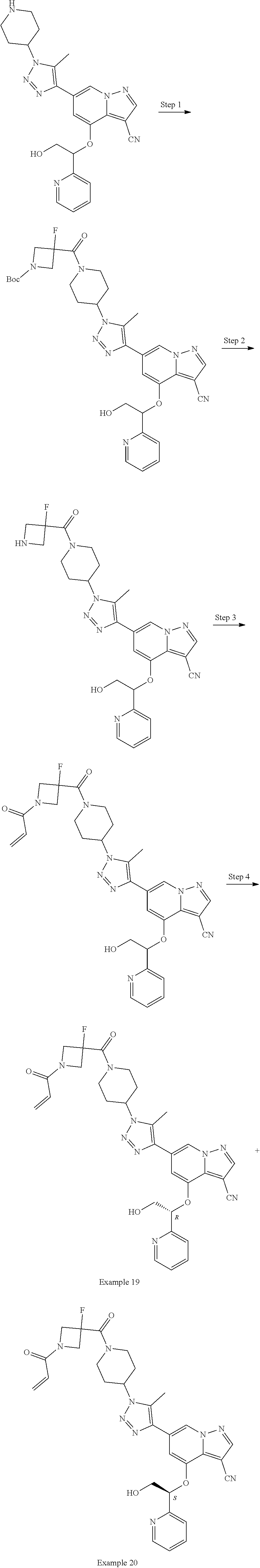

### Step 1. tert-butyl 3-[4-[4-[3-cyano-4-[2-hydroxy-1-(2-pyridyl)ethoxy]pyrazolo[1,5-a]pyridin-6-yl]-5-methyl-triazol-1-yl]piperidine-1-carbonyl]-3-fluoro-azetidine-1-carboxylate

To a solution of 1-tert-butoxycarbonyl-3-fluoro-azetidine-3-carboxylic acid (296 mg, 1.35 mmol, 1.2 eq), DIEA (726 mg, 5.62 mmol, 5.0 eq) in DMF (10 mL) was added HATU (427 mg, 1.12 mmol, 1.0 eq), and then 4-[2-hydroxy-1-(2-pyridyl)ethoxy]-6-[5-methyl-1-(4-piperidyl)triazol-4-yl]pyrazolo[1,5-a]pyridine-3-carbonitrile HCl salt (500 mg, 1.12 mmol, 1.0 eq). The resulting solution was stirred at rt for 30 min, diluted with EtOAc (90 mL), washed with brine (30 mL×2), dried over Na₂SO₄ and concentrated. The crude product was purified by Prep-TLC (DCM/MeOH=15/1) to give a white solid (300 mg, 52% yield). LCMS m/z=646.2 (M+1).

### Step 2. 6-[1-[1-(3-fluoroazetidine-3-carbonyl)-4-piperidyl]-5-methyl-triazol-4-yl]-4-[2-hydroxy-1-(2-pyridyl)ethoxy]pyrazolo[1,5-a]pyridine-3-carbonitrile

To a solution of tert-butyl 3-[4-[4-[3-cyano-4-[2-hydroxy-1-(2-pyridyl)ethoxy]-pyrazolo[1,5-a]pyridin-6-yl]-5-methyl-triazol-1-yl]piperidine-1-carbonyl]-3-fluoro-azetidine-1-carboxylate (300 mg, 2.04 mmol, 1.0 eq) in DCM (10 mL) was added an HCl/EtOAc solution (10 mL, 2M), and stirred at rt for 2 h. The reaction mixture was then concentrated to dryness to give a white solid (200 mg) as the HCl salt. LCMS m/z=546.2 (M+1).

### Step 3. 6-[1-[1-(3-fluoro-1-prop-2-enoyl-azetidine-3-carbonyl)-4-piperidyl]-5-methyl-triazol-4-yl]-4-[2-hydroxy-1-(2-pyridyl)ethoxy]pyrazolo[1,5-a]pyridine-3-carbonitrile

To a solution of 6-[1-[1-(3-fluoroazetidine-3-carbonyl)-4-piperidyl]-5-methyl-triazol-4-yl]-4-[2-hydroxy-1-(2-pyridyl)ethoxy]pyrazolo[1,5-a]pyridine-3-carbonitrile (200 mg, 0.37 mmol, 1.0 eq) and TEA (111 mg, 1.10 mmol, 3.0 eq) in DCM (5 mL) was added prop-2-enoyl chloride (33 mg, 0.37 mmol, 1.0 eq) at 0° C. The reaction mixture was stirred at rt for 15 min, then was diluted with EtOAc (40 mL) and washed with brine (15 mL×3). The organic layer was dried over Na₂SO₄ and concentrated. The crude product was purified by prep-TLC (DCM/MeOH=10/1) to give a yellow solid (90 mg, 50% yield). LCMS m/z=600.5 (M+1).

### Step 4. Example 19 and Example 20

(R)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile and (S)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile. 6-[1-[1-(3-Fluoro-1-prop-2-enoyl-azetidine-3-carbonyl)-4-piperidyl]-5-methyl-triazol-4-yl]-4-[2-hydroxy-1-(2-pyridyl)ethoxy]pyrazolo[1,5-a]pyridine-3-carbonitrile was separated by Prep-Chiral-HPLC (YMC CHIRAL ART Amylose-C NEO, 250×30 5 μm, DCXA-102; gradient elution of 1.0 ml-40% Hexane-60% EtOH-sb-30 min) to give (R)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile (peak 1, 33.9 mg, 38% yield) as a white solid; LCMS m/z=600.5 (M+H); ¹H NMR (400 MHz, DMSO-d6) δ=8.68 (d, J=0.8 Hz, 1H), 8.66 (s, 1H), 8.59 (dd, J=4.0 Hz, 0.8 Hz, 1H), 7.84 (td, J=7.6 Hz, 1.6 Hz, 1H), 7.63 (d, J=7.6 Hz, 1H), 7.37-7.33 (m, 1H), 7.29 (s, 1H), 6.39-6.30 (m, 1H), 6.15 (dd, J=17.2 Hz, 2.0 Hz, 1H), 5.75-5.72 (m, 2H), 4.95-4.75 (m, 2H), 4.59-4.56 (m, 2H), 4.43-4.30 (m, 1H), 4.30-4.24 (m, 1H), 3.98-3.96 (m, 2H), 3.73-3.70 (m, 1H), 3.43-3.42 (m, 1H), 3.05-2.99 (m, 1H), 2.49 (s, 3H), 2.15-2.06 (m, 3H), 2.01-1.95 (m, 1H) and (S)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile as a white solid (peak 2, 32.5 mg, 37% yield); LCMS m/z=600.4 (M+H); ¹H NMR (400 MHz, DMSO-d6) δ=8.68 (d, J=0.8 Hz, 1H), 8.66 (s, 1H), 8.60-8.58 (m, 1H), 7.84 (td, J=7.6 Hz, 1.6 Hz, 1H), 7.64 (d, J=8.0 Hz, 1H), 7.37-7.33 (m, 1H), 7.29 (s, 1H), 6.39-6.30 (m, 1H), 6.15 (dd, J=17.2 Hz, 2.0 Hz, 1H), 5.75-5.72 (m, 2H), 4.78-4.76 (m, 2H)), 4.60-4.54 (m, 2H), 4.46-4.43 (m, 1H), 4.30-4.24 (m, 1H), 3.97-3.73 (m, 2H), 3.73-3.70 (m, 1H), 3.50-3.44 (m, 1H), 3.05-2.99 (m, 1H), 2.49 (s, 3H), 2.15-2.06 (m, 3H), 2.01-1.91 (m, 1H).

### Example 21. (R)-6-(1-(1-(1-acryloyl-3-methoxyazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile

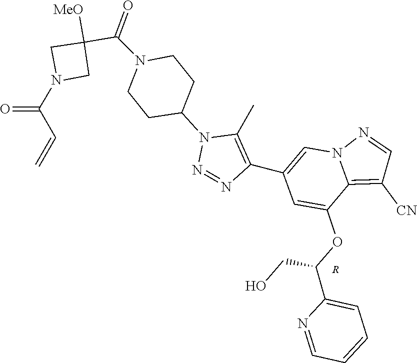

Example 21 was made by methods analogous to those described above.

LCMS m/z=612.5 (M+H); ¹H NMR (400 MHz, DMSO-d6) δ=8.68 (s, 1H), 8.67 (s, 1H), 8.59 (d, J=4.8 Hz, 1H), 7.86-7.82 (m, 1H), 7.63 (d, J=8.0 Hz, 1H), 7.37-7.34 (m, 1H), 7.28 (s, 1H), 6.40-6.30 (m, 1H), 6.13 (dd, J=16.8 Hz, 2.0 Hz 1H), 5.73-5.69 (m, 2H), 5.17 (brs, 1H), 4.75-4.64 (m, 2H), 4.52-4.49 (m, 1H), 4.40-4.27 (m, 2H), 4.00-3.96 (m, 3H), 3.86-3.82 (m, 1H), 3.28-3.26 (m, 1H), 3.24 (s, 3H), 3.01-2.95 (m, 1H), 2.46 (s, 3H), 2.12-2.04 (m, 3H), 1.96-1.92 (m, 1H).

### Example 22. (S)-6-(1-(1-(1-acryloyl-3-methoxyazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile

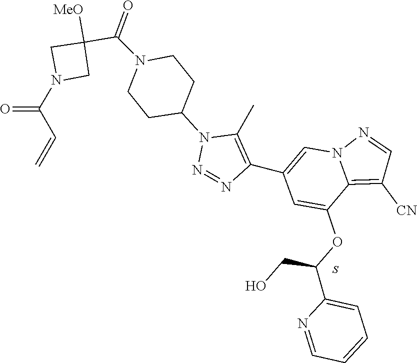

Example 22 was made by methods analogous to those described above.

LCMS m/z=612.5 (M+H); ¹H NMR (400 MHz, DMSO-d6) δ=8.68 (s, 1H), 8.66 (s, 1H), 8.59 (d, J=3.6 Hz, 1H), 7.86-7.81 (m, 1H), 7.63 (d, J=8.0 Hz, 1H), 7.37-7.34 (m, 1H), 7.28 (s, 1H), 6.40-6.29 (m, 1H), 6.13 (dd, J=16.8 Hz, 1.6 Hz 1H), 5.72-5.69 (m, 2H), 5.20 (brs, 1H), 4.75-4.63 (m, 2H), 4.52-4.49 (m, 1H), 4.39-4.27 (m, 2H), 4.00-3.96 (m, 3H), 3.85-3.82 (m, 1H), 3.28-3.26 (m, 1H), 3.23 (s, 3H), 3.01-2.94 (m, 1H), 2.45 (s, 3H), 2.12-2.04 (m, 3H), 1.98-1.92 (m, 1H).

### Example 23. (E)-6-(1-(1-(1-(4-(dimethylamino)but-2-enoyl)-3-methoxyazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile

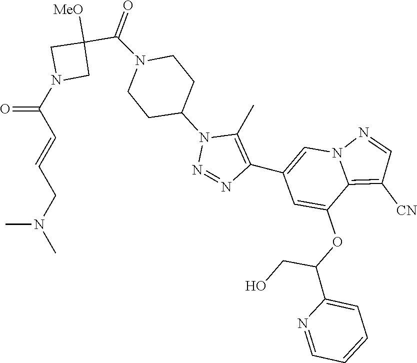 [SMILES: COC1(C(=O)N2CCC(n3nnc(-c4cc(OC(CO)c5ccccn5)c5c(C#N)cnn5c4)c3C)CC2)CN(C(=O)/C=C/CN(C)C)C1]

Example 23 was made by methods analogous to those described above.

LCMS m/z=6669.5 (M+H); ¹H NMR (400 MHz, DMSO-d6) δ=8.68 (s, 1H), 8.66 (s, 1H), 8.59 (d, J=4.4 Hz, 1H), 7.86-7.82 (m, 1H), 7.63 (d, J=7.6 Hz, 1H), 7.36-7.33 (m, 1H), 7.28 (s, 1H), 6.64-6.57 (m, 1H), 6.17-6.10 (m, 1H), 5.72 (t, J=5.6 Hz, 1H), 5.17 (t, J=5.6 Hz, 1H), 4.74-4.61 (m, 2H), 4.52-4.48 (m, 1H), 4.38-4.25 (m, 2H), 4.01-3.92 (m, 3H), 3.86-3.82 (m, 1H), 3.28-3.27 (m, 1H), 3.23 (s, 3H), 3.03-2.94 (m, 3H), 2.45 (s, 3H), 2.15 (s, 6H), 2.13-2.03 (m, 3H), 1.98-1.92 (m, 1H).

### Example 24. 4-(2-hydroxy-1-(pyridin-2-yl)ethoxy)-6-(1-(1-(3-methoxy-1-propioloylazetidine-3-carbonyl)piperidin-4-yl)-5-methyl-1H-1,2,3-triazol-4-yl)pyrazolo[1,5-a]pyridine-3-carbonitrile

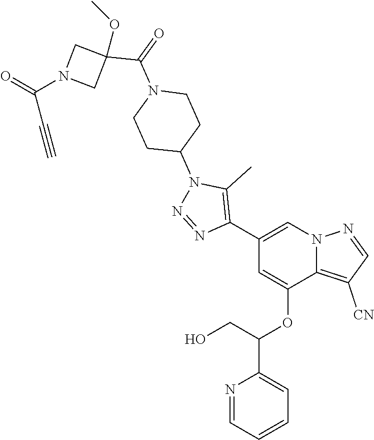 [SMILES: C#CC(=O)N1CC(OC)(C(=O)N2CCC(n3nnc(-c4cc(OC(CO)c5ccccn5)c5c(C#N)cnn5c4)c3C)CC2)C1]

Example 24 was made by methods analogous to those described above.

LCMS m/z=610.4 (M+H); ¹H NMR (400 MHz, DMSO-d6) δ=8.68 (s, 1H), 8.65 (s, 1H), 8.59 (d, J=4.4 Hz, 1H), 7.84 (t, J=8.0 Hz, 1H), 7.63 (d, J=8.0 Hz, 1H), 7.36-7.33 (m, 1H), 7.28 (s, 1H), 5.72 (t, J=6.0 Hz, 1H), 5.16 (t, J=5.6 Hz, 1H), 4.77-4.59 (m, 2H), 4.51-4.48 (m, 2H), 4.41-4.23 (m, 2H), 4.03-3.93 (m, 3H), 3.84-3.81 (m, 1H), 3.28-3.24 (m, 1H), 3.24 (s, 3H), 3.01-2.95 (m, 1H), 2.45 (s, 3H), 2.12-2.03 (m, 3H), 1.99-1.90 (m, 1H).

### Example 25. (R)-6-(1-(7-acryloyl-7-azaspiro[3.5]nonan-2-yl)-1H-pyrazol-4-yl)-4-(1-(5-fluoropyridin-2-yl)-2-hydroxyethoxy)pyrazolo[1,5-a]pyridine-3-carbonitrile

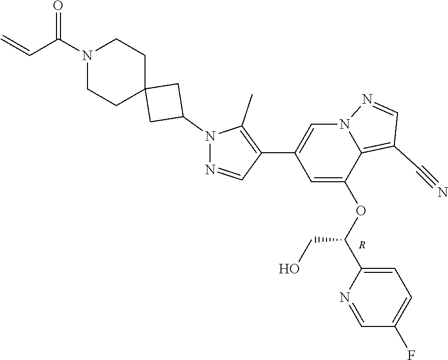

Example 25 was made by methods analogous to those described above.

LCMS m/z=542.3 (M+H); ¹H NMR (400 MHz, DMSO-d6) δ=8.87 (s, 1H), 8.64-8.59 (m, 1H), 8.57 (s, 1H), 8.46 (s, 1H), 8.08 (s, 1H), 7.80-7.78 (m, 2H), 7.42 (s, 1H), 6.87-6.77 (m, 1H), 6.08 (dd, J=16.8 Hz, 2.8 Hz, 1H), 5.98-5.95 (m, 1H), 5.67-5.63 (m, 1H), 5.12 (brs, 1H), 4.95-4.87 (m, 1H), 4.05-4.01 (m, 1H), 3.95-3.90 (m, 1H), 3.55-3.45 (m, 4H), 2.47-2.41 (m, 2H), 2.32-2.21 (m, 2H), 1.72-1.50 (m, 4H).

### Example 26. (R)-4-(1-(5-fluoropyridin-2-yl)-2-hydroxyethoxy)-6-(1-(7-propionyl-7-azaspiro[3.5]nonan-2-yl)-1H-pyrazol-4-yl)pyrazolo[1,5-a]pyridine-3-carbonitrile

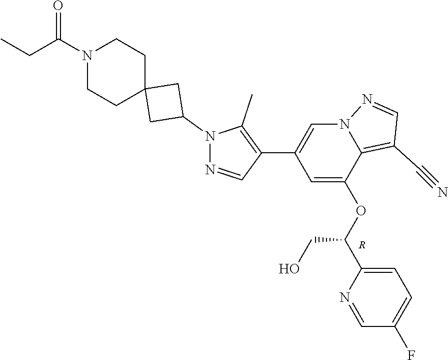

Example 26 was made by methods analogous to those described above.

LCMS m/z=544.3 (M+H); ¹H NMR (400 MHz, DMSO-d6) δ=8.87 (s, 1H), 8.62-8.60 (m, 1H), 8.57 (s, 1H), 8.45 (s, 1H), 8.08 (s, 1H), 7.80-7.78 (m, 2H), 7.42 (s, 1H), 6.00-5.94 (m, 1H), 5.13 (brs, 1H), 4.95-4.85 (m, 1H), 4.05-4.01 (m, 1H), 3.95-3.90 (m, 1H), 3.48-3.38 (m, 4H), 2.45-2.40 (m, 2H), 2.37-2.22 (m, 4H), 1.69-1.52 (m, 4H), 0.98 (t, J=6.8 Hz, 3H).

### Example 27. (S)-6-(1-(7-acryloyl-7-azaspiro[3.5]nonan-2-yl)-1H-pyrazol-4-yl)-4-(1-(5-fluoropyridin-2-yl)-2-hydroxyethoxy)pyrazolo[1,5-a]pyridine-3-carbonitrile

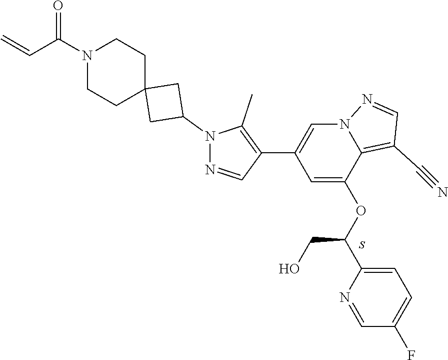

Example 27 was made by methods analogous to those described above.

LCMS m/z=543.3 (M+H); ¹H NMR (400 MHz, DMSO-d6) δ=8.88 (s, 1H), 8.62-8.60 (m, 1H), 8.57 (s, 1H), 8.46 (s, 1H), 8.08 (s, 1H), 7.81-7.76 (m, 2H), 7.42 (s, 1H), 6.87-6.77 (m, 1H), 6.08 (dd, J=16.8 Hz, 2.4 Hz, 1H), 5.98-5.95 (m, 1H), 5.65 (d, J=11.6 Hz, 1H), 5.13 (brs, 1H), 4.95-4.87 (m, 1H), 4.05-4.01 (m, 1H), 3.95-3.90 (m, 1H), 3.54-3.46 (m, 4H), 2.47-2.41 (m, 2H), 2.28-2.24 (m, 2H), 1.67-1.59 (m, 4H).

### Example 28. (S)-4-(1-(5-fluoropyridin-2-yl)-2-hydroxyethoxy)-6-(1-(7-propionyl-7-azaspiro[3.5]nonan-2-yl)-1H-pyrazol-4-yl)pyrazolo[1,5-a]pyridine-3-carbonitrile

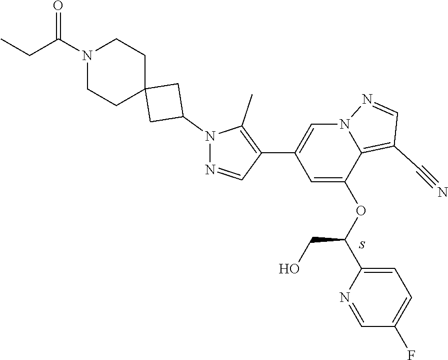

Example 28 was made by methods analogous to those described above.

LCMS m/z=544.3 (M+H); ¹H NMR (400 MHz, DMSO-d6) δ=8.87 (s, 1H), 8.62-8.59 (m, 1H), 8.56 (s, 1H), 8.45 (s, 1H), 8.08 (s, 1H), 7.80-7.65 (m, 2H), 7.42 (s, 1H), 5.98-5.94 (m, 1H), 5.12 (t, J=6.0 Hz, 1H), 4.95-4.86 (m, 1H), 4.06-4.02 (m, 1H), 3.96-3.90 (m, 1H), 3.49-3.35 (m, 4H), 2.49-2.40 (m, 2H), 2.33-2.22 (m, 4H), 1.69-1.55 (m, 4H), 0.98 (t, J=7.2 Hz, 3H).

### Example 29. 6-(1-(1-(1-acryloyl-3-fluoroazetidine-3-carbonyl)piperidin-4-yl)-5-methyl-1H-1,2,3-triazol-4-yl)-4-(2-(dimethylamino)-ethoxy)pyrazolo[1,5-a]pyridine-3-carbonitrile

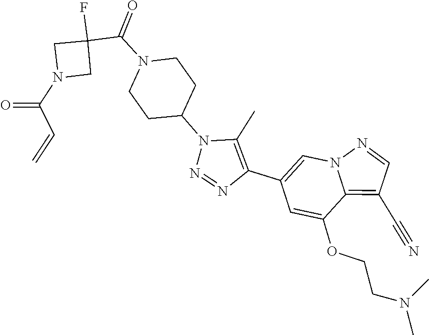 [SMILES: C=CC(=O)N1CC(F)(C(=O)N2CCC(n3nnc(-c4cc(OCCN(C)C)c5c(C#N)cnn5c4)c3C)CC2)C1]

Example 29 was made by methods analogous to those described above.

LCMS m/z=550.5 (M+H); ¹H NMR (400 MHz, DMSO-d6) δ=8.70 (s, 1H), 8.62 (s, 1H), 7.33 (s, 1H), 6.39-6.33 (m, 1H), 6.15 (dd, J=16.8 Hz, 1.6 Hz, 1H), 5.73 (dd, J=10.4 Hz, 1.6 Hz, 1H), 4.99-4.77 (m, 2H), 4.60-4.44 (m, 3H), 4.39 (t, J=5.2 Hz, 2H), 4.31-4.22 (m, 1H), 3.73 (d, J=12.4 Hz, 1H), 3.48-3.45 (m, 1H), 3.04 (t, J=12.8 Hz, 1H), 2.80-2.76 (m, 2H), 2.57 (s, 3H), 2.29 (s, 6H), 2.12-1.98 (m, 4H).

### Example 30. 4-(2-(dimethylamino) ethoxy)-6-(1-(1-(3-fluoro-1-propionylazetidine-3-carbonyl)piperidin-4-yl)-5-methyl-1H-1,2,3-triazol-4-yl)pyrazolo[1,5-a]pyridine-3-carbonitrile

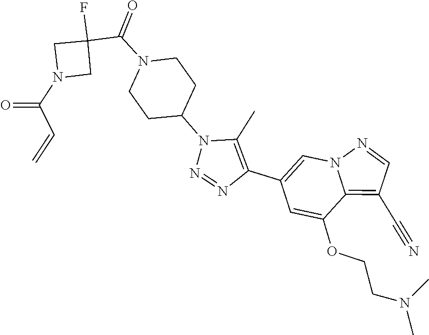 [SMILES: C=CC(=O)N1CC(F)(C(=O)N2CCC(n3nnc(-c4cc(OCCN(C)C)c5c(C#N)cnn5c4)c3C)CC2)C1]

Example 30 was made by methods analogous to those described above.

LCMS m/z=552.6 (M+H); ¹H NMR (400 MHz, DMSO-d6) δ=8.70 (s, 1H), 8.62 (s, 1H), 7.33 (s, 1H), 4.85-4.70 (m, 2H), 4.57-4.45 (m, 3H), 4.39 (t, J=5.2 Hz, 2H), 4.19-4.10 (m, 1H), 3.73 (d, J=13.2 Hz, 1H), 3.45-3.39 (m, 1H), 3.07-3.00 (m, 1H), 2.80 (t, J=5.2 Hz, 2H), 2.57 (s, 3H), 2.30 (s, 6H), 2.14-1.97 (m, 6H), 0.97 (t, J=7.2 Hz, 3H).

### Example A—Biological Data

### Kinase Assays

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. Streptavidin-coated magnetic beads were treated with biotinylated small molecule ligands for 30 minutes at room temperature to generate affinity resins for kinase assays. The liganded beads were blocked with excess biotin and washed with blocking buffer SeaBlock (Pierce), 1% BSA, 0.05% Tween 20, 1 mM DTT to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT).

Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Binding Constants (Kds)

Binding constants were calculated with a standard dose-response curve using the Hill equation:

$$Response = Background + ( Signal - Background ) / ( 1 + ( \text{?} Kd \text{?} \hat{} ( Hill Slope ) / \text{?} Dose \text{?} \hat{} ( Hill Slope ) ) ) .$$

The Hill Slope was set to −1. Curves were fitted using a non-linear least square fit with the Levenberg-Marquardt algorithm.

### Cell Viability Assays

Cell lines used for cell viability assays are shown in Table 2.

TABLE 2
Cell Lines

| | Mutation or | |
|---|---|---|
| Cell line | Fusion | Source |
| Ba/F3 BCR-FGFR1 | FGFR1-fusion | Advanced Cellular |
| | | Dynamics, (Seattle, WA) |
| Ba/F3 BCR-FGFR2 | FGFR2-fusion | Advanced Cellular |
| | | Dynamics, (Seattle, WA) |
| Ba/F3 FGFR3-BAIAP2L1 | FGFR3-fusion | Advanced Cellular |
| | | Dynamics, (Seattle, WA) |
| Ba/F3 ETV5-FGFR4 | FGFR4-fusion | Kyinno (Waltham, MA) |
| RT112/84 | FGFR3-fusion | American Type Culture |
| | | Collection (Manassas, VA) |
| UM-UC-14 | FGFR3(S249C) | Sigma, (St. Louis, MO) |
| KG-1 | FGFR1-fusion | American Type Culture |
| | | Collection (Manassas, VA) |

The results of FGFR assay testing for cell viability are shown below in Table 3.

### Ba/F3 Cell Viability Assays

Experimental Purpose: Recombinant kinase fusions are transduced into parental Ba/F3, which becomes dependent upon this constitutive kinase activity for IL3-independent survival. Inhibition of kinase activity leads to cell death, which is monitored using CellTiter-Glo® 2.0 (Promega) which measures intracellular ATP concentration that in turn serves as a marker for viability. BCR-FGFR1 Ba/F3, BCR-FGFR2 and FGFR3-BAIAP2L1 Ba/F3 were obtained from Advanced Cellular Dynamics (Seattle, WA). ETV6-FGFR4 was obtained from Kyinno (Waltham, MA).

Cell Viability Assay Procedure: Cell Titer-Glo® 2.0 Luminescent cell viability assay reagent was purchased from Promega (Madison, WI). Ba/F3 cell lines were cultured in RPMI1640 media supplemented with 10% fetal bovine serum. Cultures were maintained at 37° C. in a humidified atmosphere of 5% CO₂ and 95% air.

Cells were plated in 96-well clear bottom/white plates (Corning #3903) at 10,000 cells/well in 100 μl of media, incubated overnight. The next day, test compound DMSO stock solutions were made at 10 mM and 2 μM final concentration. Compounds were then added to cells in a 9-dose, 10-fold dilution series starting at 30 μM with an HP 300e Digital Dispenser (each dose was applied in triplicate). DMSO was backfilled to each well up to 301 nL total volume of test compound+DMSO, and a total of 301 nL DMSO was added to a control/no test compound well in triplicate. The cells in cell culture plates were incubated with the compounds at 37° C. and 5% CO₂ for 48 hours. Then 50 μl of Cell Titer Glo 2.0 reagent was added to each well of the cell culture plates. The contents were covered from light and mixed on an orbital shaker at room temperature for 10 min. Luminescence was recorded by a Synergy H1 Microplate Reader (Biotek, Winooski, VT). Cells were assessed as a percentage of DMSO only treated control cells. Curves were plotted and IC₅₀ values were calculated using the GraphPad Prism 8 program based on a sigmoidal dose-response equation (4 parameter).

The results of Ba/F3 assay testing for cell viability are shown below in Table 4.

### RT112/84, UM-UC-14 and KG-1 Cancer Cell Line Cell Viability Assays

Experimental Purpose: To detect the change of intracellular ATP by Cell Titer-Glo® and to evaluate the inhibitory effect of the compounds on cancer cell lines by determining the in vitro IC₅₀ value of the compounds.

Cell Titer-Glo® 2.0 Luminescent cell viability assay reagent was purchased from Promega (Madison, WI). RT112/84 and KG-1 cell lines were purchased from American Type Culture Collection (Manassas, VA). UM-UC-14 cell line was purchased from Sigma (St. Louis, MO). RT112/84, UM-UC-14, and KG-1 cells were cultured in RPMI1640 media supplemented with 10% fetal bovine serum. Cultures were maintained at 37° C. in a humidified atmosphere of 5% CO₂ and 95% air.

Cell Viability Assay Procedure: Cells were plated in 96-well clear bottom/white plates (Corning #3903) at 10,000 cells/well in 100 μl of media, incubated overnight. The next day, test compound DMSO stock solutions were made at 10 mM and 2 μM final concentration.

Compounds were then added to cells in a 9-dose, 10-fold dilution series starting at 30 μM with an HP 300e Digital Dispenser (each dose was applied in triplicate). DMSO was backfilled to each well up to 301 nL total volume of test compound+DMSO, and a total of 301 nL DMSO was added to a control/no test compound well in triplicate. The cells in cell culture plates were incubated with the compounds at 37° C. and 5% CO₂ for 72 hours. Then 50 μl of Cell Titer Glo 2.0 reagent was added to each well of the cell culture plates. The contents were covered from light and mixed on an orbital shaker at room temperature for 10 min.

Luminescence was recorded by a Synergy H1 Microplate Reader (Biotek, Winooski, VT). Cells were assessed as a percentage of DMSO only treated control cells. Curves were plotted and IC₅₀ values were calculated using the GraphPad Prism 8 program based on a sigmoidal dose-response equation (4 parameter).

The results of RT112/84, UM-UC-14 and KG-1 assay testing for cell viability are shown below in Table 5.

TABLE 3
FGFR In Vitro Binding Assay Data

| | | | | | FGFR3 |
|---|---|---|---|---|---|
| | FGFR1 | FGFR2 | FGFR3 | FGFR4 | (V555M) |
| Example | (K_{d} nM) | (K_{d} nM) | (K_{d} nM) | (K_{d} nM) | (K_{d} nM) |
| 1 | B | A | A | B | A |
| 2 | C | C | C | D | C |
| 3 | A | | A | | |
| 4 | A | | A | | |
| 5 | D | | C | | |
| 6 | B | | A | | |
| 7 | C | B | B | C | C |
| 8 | A | A | A | A | A |
| 9 | A | A | A | A | A |
| 10 | C | C | C | S | C |
| 11 | C | B | B | C | B |
| 12 | C | C | B | D | C |
| 13 | A | A | A | A | A |
| 14 | B | A | A | B | B |
| 15 | A | A | A | A | A |
| 16 | B | A | A | A | A |
| 17 | D | C | B | D | C |
| 18 | A | A | A | A | A |
| 19 | D | C | B | D | C |
| 20 | A | A | A | A | A |
| 21 | D | C | B | D | C |
| 22 | A | A | A | A | A |
| 23 | A | A | A | A | A |
| 24 | A | A | A | A | A |
| 25 | B | B | B | C | C |
| 26 | B | B | B | C | C |
| 27 | A | A | A | A | A |
| 28 | A | A | A | A | A |

TABLE 4
Ba/F3 Cell Data

| | FGFR1 | FGFR2 | FGFR3 | FGFR4 |
|---|---|---|---|---|
| Example | (K_{d} nM) | (K_{d} nM) | (K_{d} nM) | (K_{d} nM) |
| 1 | C | B | A | C |
| 2 | D | D | C | D |
| 3 | A | A | A | B |
| 4 | B | B | A | C |
| 5 | D | D | C | D |
| 6 | C | C | A | C |
| 7 | D | C | B | C |
| 8 | B | B | A | C |
| 9 | C | B | A | C |
| 10 | D | D | C | D |
| 11 | D | D | C | D |
| 12 | D | D | C | D |
| 13 | C | B | A | C |
| 14 | C | C | A | C |
| 15 | B | B | A | C |
| 18 | D | D | C | D |
| 20 | D | D | A | D |
| 21 | D | D | D | D |
| 22 | D | D | A | D |
| 23 | D | D | C | D |
| 27 | A | A | A | B |
| 28 | B | A | A | B |

TABLE 5
Cancer Cell Data

| | KG-1 | KatoIIII | RT112/84 |
|---|---|---|---|
| Example | (IC₅₀ nM) | (IC₅₀ nM) | (IC₅₀ nM) |
| 1 | D | B | B |
| 2 | D | C | D |
| 3 | A | A | A |
| 8 | B | B | B |
| 9 | D | C | C |
| 13 | A | B | B |
| 14 | C | C | C |
| 18 | D | D | D |
| 20 | C | D | B |
| 22 | D | D | D |

In Tables 3, 4 and 5, A=0.1-100 nM; B≥100-250 nM; and C≥250-1000 nM; and D≥1000 nM.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of the formula: 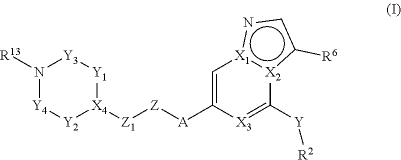
wherein
A is pyrazole, triazole, thiadiazole or oxadiazole, substituted with R¹ and R^{1A},
R¹ is hydrogen or C₁-C₃ alkyl;
R^{1A} is hydrogen, halo, CN, or C₁-C₃ alkyl optionally substituted with one or more substituents independently selected from halo, OH, and OCH₃;
X₁ and X₂ are independently selected from N and C, wherein when one of X₁ or X₂ is N the other is C;
X₃ is N or CH;
X₄ is N or C—R⁹;
Y is NH, O, S or a bond; Y₁ is a bond, CHR⁷, CH₂—CHR⁷, CHR⁷—CH₂, CF₂, CH₂—CF₂ or CF₂—CH₂;
Y₂ is a bond, CHR³, CH₂—CHR³, CHR³—CH₂, CF₂, CH₂—CF₂ or CF₂—CH₂;
Y₃ is CR⁴R⁵ or CF₂;
Y₄ is CR³R⁴, or CF₂;
Z is a bond, CHR^{9A}, CR⁴R^{4A}, CR⁴R^{4A}—CH₂, CH₂—CR⁴R^{4A}, cyclobutyl, cyclopentyl, cyclohexyl, bicyclo(1.1.1) pentane, bicyclo(2.1.1) hexane, azetidine, pyrrolidine or piperidine;
Z₁ is a bond when Z is a bond, CR⁴R^{4A}, CR⁴R^{4A}—CH₂, CH₂—CR⁴R^{4A}, cyclobutyl, cyclopentyl, cyclohexyl, bicyclo(1.1.1) pentane, bicyclo(2.1.1) hexane, azetidine, pyrrolidine or piperidine, or Z₁ is CH₂ or CH₂—CH₂ when Z is CHR^{9A};
Z₂ is a bond, C(O), SO₂ or —NR⁴C(O);
Z₃ is a bond, C(O), SO₂ or —NR⁴C(O); R² is C₁-C₅ alkyl or R⁸, wherein C₁-C₅ alkyl is optionally substituted with one or more substituents independently selected from halo, OH, CN, oxo, —OC₁-C₄ alkyl, —OC₃-C₅ cycloalkyl, —Z₂—R¹¹ and R¹⁰, wherein C₁-C₄ alky and C₃-C₅ cycloalkyl are optionally substituted with one or more substituents independently selected from halo, OH, OCH₃, methylamine, N,N-dimethylamine and CN;
R³ is hydrogen, F, OH, OCH₃, C₁-C₃ alkyl, cyclopropyl, or one R³ is fused with R⁵ or R⁷ to form CH₂, CH₂—CH₂ or CH₂OCH₂;
R⁴ is hydrogen or C₁-C₃ alkyl;
R^{4A} is hydrogen, halo, OH, or C₁-C₃ alkyl;
R⁵ is hydrogen, F, OH, OCH₃, C₁-C₃ alkyl, cyclopropyl, or is fused with one R³ to form CH₂, CH₂—CH₂ or CH₂OCH₂;
R⁶ is hydrogen, halo, C₁-C₅alkyl, CN, 3-6 membered cycloalkyl, 4-6 membered heterocycloalkyl, 5-6 membered aryl or 5-6 membered heteroaryl, wherein 3-6 membered cycloalkyl, 4-6 membered heterocycloalkyl, 5-6 membered aryl and 5-6 membered heteroaryl are optionally substituted with one or more substituents independently selected from halo, methyl, halomethyl, OH or OCH₃ and wherein C₁-C₅ alkyl is optionally substituted with one or more substituents independently selected from halo, OH and OCH₃;
R⁷ is hydrogen, F, OH, OCH₃, C₁-C₃ alkyl or is fused with one R³ to form CH₂, CH₂—CH₂ or CH₂OCH₂;
R⁸ is 3-6 membered cycloalkyl, 4-6 membered heterocycloalkyl, 5-6 membered aryl or 5-6 membered heteroaryl, optionally fused or substituted with R^{8A},
R^{8A} is 3-6 membered cycloalkyl, 4-6 membered heterocycloalkyl, 5-6 membered aryl or 5-6 membered heteroaryl;
R⁹ is hydrogen, C₁-C₃ alkyl, or is fused with R^{9A} to form CH₂ or CH₂—CH₂;
R¹⁰ is 3-6 membered cycloalkyl, 4-6 membered heterocycloalkyl, 5-6 membered aryl or 5-6 membered heteroaryl, optionally fused or substituted with R^{8A};
R¹¹ is C₁-C₄ alkyl, NH₂, NHC₁-C₃ alkyl, NHC₃-C₅ cycloalkyl or N(C₁-C₃ alkyl)₂, wherein C₁-C₄ alkyl, C₁-C₃ alkyl and C₃-C₅ cycloalkyl are optionally substituted with one or more substituents independently selected from halo, OH, OCH₃, methylamine, N,N-dimethylamine and CN; R¹² is C₁-C₄ alkyl, C₃-C₅ cycloalkyl, NH₂, NHC₁-C₃ alkyl, NHC₃-C₅ cycloalkyl or N(C₁-C₃ alkyl)₂, wherein C₁-C₄ alky, C₁-C₃ alkyl and C₃-C₅ cycloalkyl are optionally substituted with one or more substituents independently selected from halo, OH, OCH₃, methylamine, N,N-dimethylamine and CN; and R⁸, R¹⁰ and R^{8A} are optionally substituted with one or more substituents independently selected from halo, OH, CN, —OC₁-C₄ alkyl, —OC₃-C₅ cycloalkyl and —Z₃—R¹² wherein C₁-C₄ alky and C₃-C₅ cycloalkyl are optionally substituted with one or more substituents independently selected from halo, OH, OCH₃, methylamine, N,N-dimethylamine and CN;
and R¹³ is 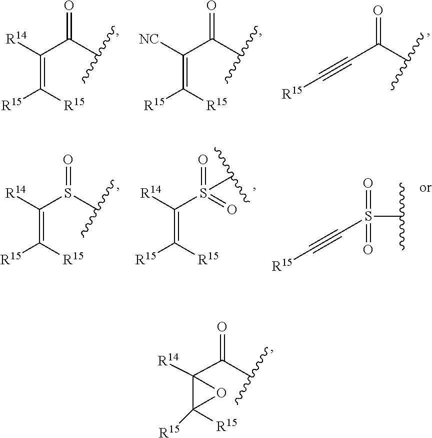
wherein each R¹⁴ and each R¹⁵ is independently H, optionally substituted C₁-C₆alkyl, or halogen;
or a pharmaceutically acceptable salt thereof.

2-28. (canceled)

29. The compound according to claim 1 of the formula: 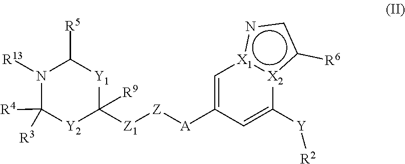
wherein
A is pyrazole, triazole, thiadiazole or oxadiazole, optionally substituted with R¹;
R¹ is C₁-C₃ alkyl;
X₁ and X₂ are independently selected from N and C, wherein when one of X₁ or X₂ is N the other is C;
Y is NH, O, or a bond; Y₁is a bond, CHR⁷, CH₂—CHR⁷ or CHR⁷—CH₂;
Y₂ is a bond, CH₂, CF₂, CHR³, CH₂—CHR³ or CHR³—CH₂;
Z is a bond, CHR^{9A}, azetidine, pyrrolidine or piperidine; Z₁ is a bond when Z is a bond, azetidine, pyrrolidine or piperidine, or Z₁ is CH₂ or CH₂—CH₂ when Z is CHR^{9A};
R² is C₁-C₅alkyl or R⁸, wherein C₁-C₅alkyl is optionally substituted with one or more substituents independently selected from OH, methoxy, halomethyl and R¹⁰;
R³ is hydrogen, C₁-C₃ alkyl, or one R³ is fused with R⁵ or R⁷ to form CH₂ or CH₂—CH₂;
R⁴ is hydrogen, or C₁-C₃ alkyl;
R⁵ is hydrogen, or is fused with one R³ to form CH₂ or CH₂—CH₂;
R⁶ is hydrogen, CH₂, CN, Cl or F;
R⁷ is hydrogen, or is fused with one R³ to form CH₂ or CH₂—CH₂;
R⁸ is 3-6 membered cycloalkyl, 5-6 membered heterocycloalkyl, 5-6 membered aryl or 5-6 membered heteroaryl, optionally fused or substituted with R^{8A}, R^{8A} is 3-6 membered cycloalkyl, 5-6 membered heterocycloalkyl, 5-6 membered aryl or 5-6 membered heteroaryl;
R⁹ is hydrogen or is fused with R^{9A} to form CH₂ or CH₂—CH₂;
R¹⁰ is 3-6 membered cycloalkyl, 5-6 membered heterocycloalkyl, 5-6 membered aryl or 5-6 membered heteroaryl, optionally fused or substituted with R^{8A}, and R⁸, R¹⁰ and R^{8A} are optionally substituted with one or more substituents independently selected from halo, CN, methyl, halomethyl, methoxy, ethyl, ethoxy, methylamine, S(O)₂CH₃, C(O)NH₂, N,N-dimethylamine and C(O)N,N-dimethylamine; or a pharmaceutically acceptable salt thereof.

30. (canceled)

31. The compound according to claim 29 of the formula: 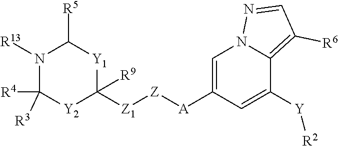
or a pharmaceutically acceptable salt thereof.

32. The compound according to claim 29 of the formula: 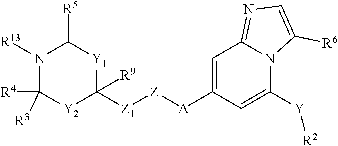
or a pharmaceutically acceptable salt thereof.

33. The compound according to claim 29, wherein A is pyrazole or triazole, optionally substituted with R¹, or a pharmaceutically acceptable salt thereof.

34. (canceled)

35. The compound according to claim 1, wherein R¹ is methyl, or a pharmaceutically acceptable salt thereof.

36. The compound according to claim 1, wherein Y is NH or O, or a pharmaceutically acceptable salt thereof.

37. (canceled)

38. The compound according to claim 29, wherein R² is C₁-C₃ alkyl optionally substituted with one, two or three substituents independently selected from OH, methoxy, halomethyl and R¹⁰, or a pharmaceutically acceptable salt thereof.

39. (canceled)

40. The compound according to claim 29, wherein R¹⁰ is 5-6 membered heterocycloalkyl or 5-6 membered heteroaryl, optionally fused with R^{8A}, or a pharmaceutically acceptable salt thereof.

41-45. (canceled)

46. The compound according to claim 1, wherein Z is a bond, azetidine or piperidine, or a pharmaceutically acceptable salt thereof.

47-48. (canceled)

49. The compound according to claim 29, wherein R³ and R⁴ are hydrogen or CH₃, or a pharmaceutically acceptable salt thereof.

50-51. (canceled)

52. The compound according to claim 29, wherein Y₁ is a bond or CH₂, or a pharmaceutically acceptable salt thereof.

53. The compound according to claim 29, wherein Y₂ is a bond or CH₂, or a pharmaceutically acceptable salt thereof.

54. The compound according to claim 1, selected from the group consisting of: 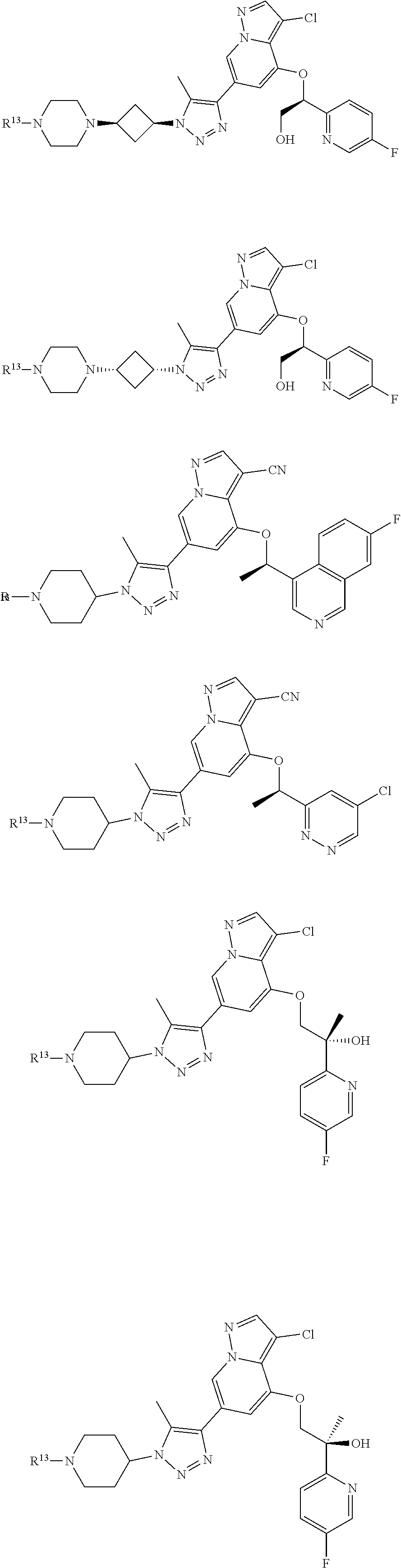 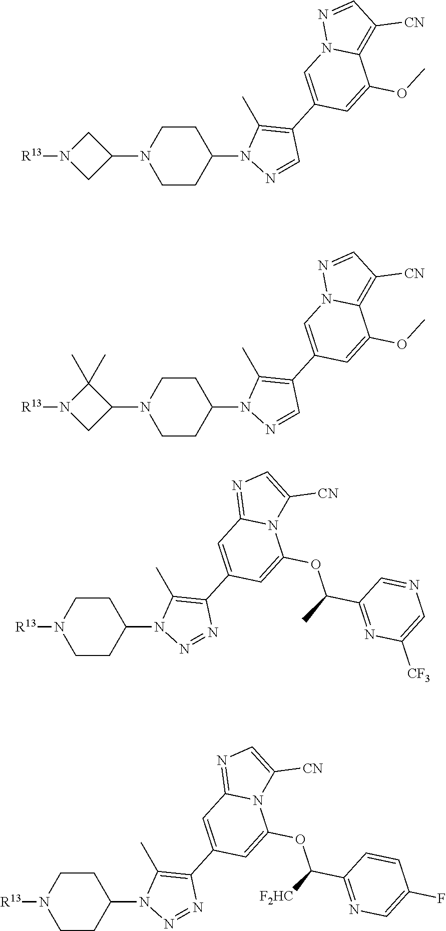 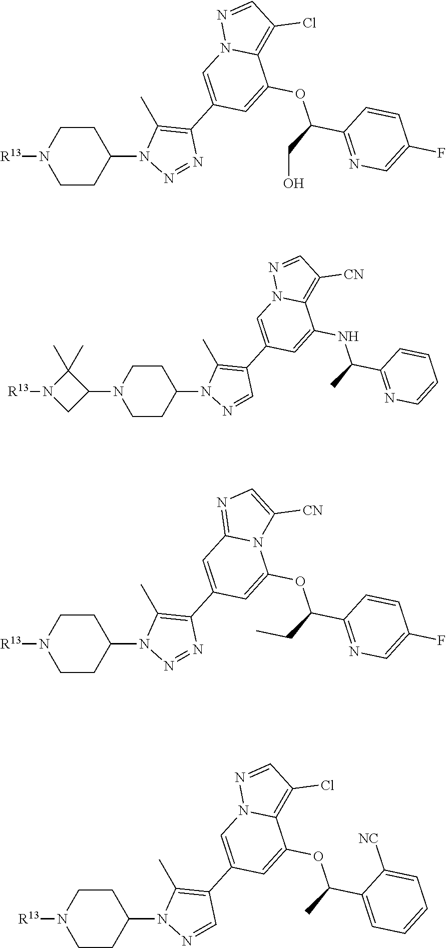 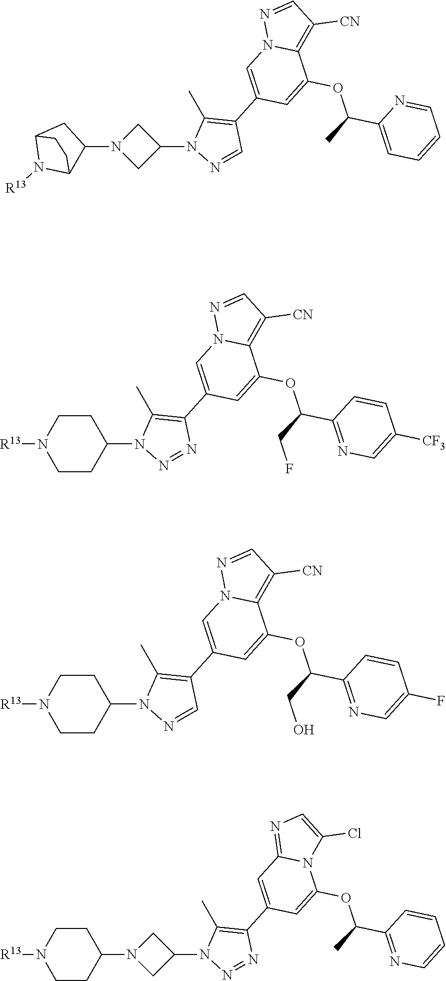 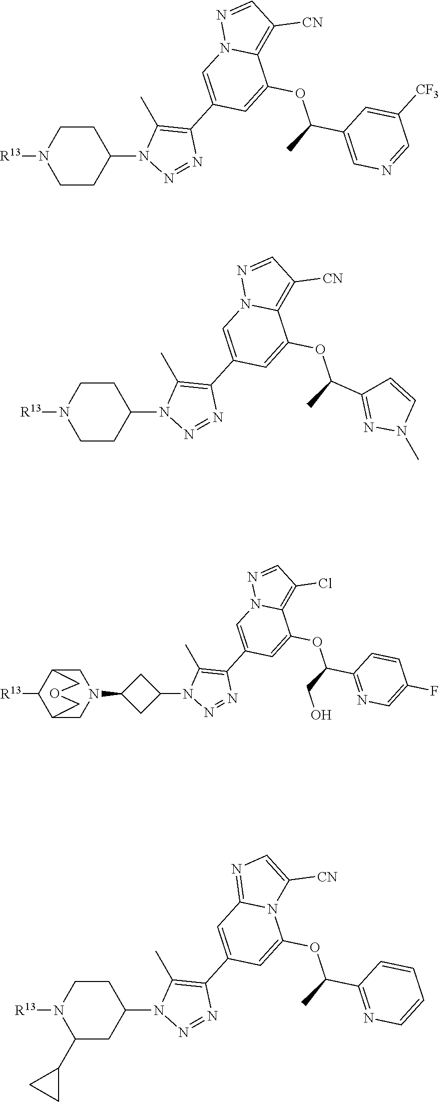 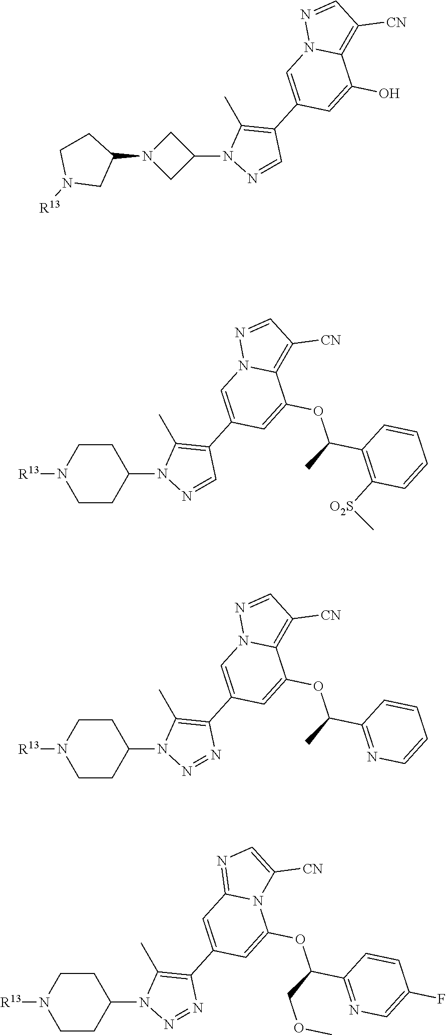 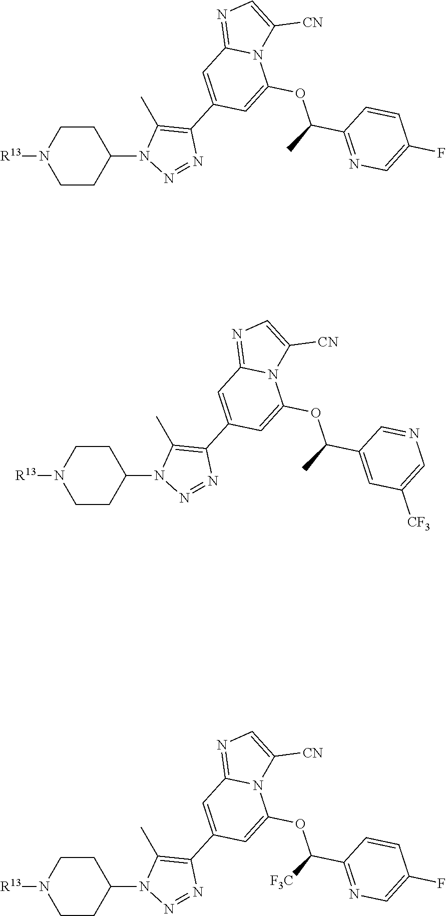
or a pharmaceutically acceptable salt thereof.

55. The compound according to claim 1, selected from the group consisting of:
4-[1-(5-Fluoro-2-pyridyl)-2-hydroxy-ethoxy]-6-[5-methyl-1-(1-prop-2-enoyl-4-piperidyl) pyrazol-4-yl]pyrazolo[1,5-a]pyridine-3-carbonitrile;
4-[1-(5-Fluoro-2-pyridyl)-2-hydroxy-ethoxy]-6-[5-methyl-1-(1-prop-2-enoyl-4-piperidyl) pyrazol-4-yl]pyrazolo[1,5-a]pyridine-3-carbonitrile;
(R)-6-(1-(7-acryloyl-7-azaspiro[3.5]nonan-2-yl)-5-methyl-1H-1,2,3-triazol-4-yl)-4-(1-(pyridin-2-yl)ethoxy)pyrazolo[1,5-a]pyridine-3-carbonitrile;
(R)-1-(2-(4-(3-chloro-4-(1-(pyridin-2-yl)ethoxy)-pyrazolo[1,5-a]pyridin-6-yl)-5-methyl-1H-1,2,3-triazol-1-yl)-7-azaspiro[3.5]nonan-7-yl)prop-2-en-1-one;
(R)-1-(2-(4-(3-chloro-4-(2-hydroxy-1-(pyridin-2-yl)ethoxy)pyrazolo[1,5-a]pyridin-6-yl)-5-methyl-1H-pyrazol-1-yl)-7-azaspiro[3.5]nonan-7-yl)prop-2-en-1-one;
(S)-1-(2-(4-(3-chloro-4-(2-hydroxy-1-(pyridin-2-yl)ethoxy)pyrazolo[1,5-a]pyridin-6-yl)-5-methyl-1H-pyrazol-1-yl)-7-azaspiro[3.5]nonan-7-yl)prop-2-en-1-one;
(R)-6-(1-(7-acryloyl-7-azaspiro[3.5]nonan-2-yl)-5-methyl-1H-pyrazol-4-yl)-4-(2-hydroxy-1-(pyridin-2-yl)ethoxy)pyrazolo[1,5-a]pyridine-3-carbonitrile;
(S)-6-(1-(7-acryloyl-7-azaspiro[3.5]nonan-2-yl)-5-methyl-1H-pyrazol-4-yl)-4-(2-hydroxy-1-(pyridin-2-yl)ethoxy)pyrazolo[1,5-a]pyridine-3-carbonitrile;
(S)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile;
(R)-6-(1-(7-acryloyl-7-azaspiro[3.5]nonan-2-yl)-5-methyl-1H-pyrazol-4-yl)-4-(1-(5-fluoropyridin-2-yl)-2-hydroxyethoxy)pyrazolo[1,5-a]pyridine-3-carbonitrile;
6-(1-(7-acryloyl-7-azaspiro[3.5]nonan-2-yl)-5-methyl-1H-pyrazol-4-yl)-4-(2-hydroxy-1-(1-methyl-1H-pyrazol-5-yl)ethoxy)pyrazolo[1,5-a]pyridine-3-carbonitrile;
(R)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile;
(S)-6-(1-(7-acryloyl-7-azaspiro[3.5]nonan-2-yl)-5-methyl-1H-1,2,3-triazol-4-yl)-4-(1-(5-fluoropyridin-2-yl)-2-hydroxyethoxy)pyrazolo[1,5-a]pyridine-3-carbonitrile;
6-(1-(7-acryloyl-7-azaspiro[3.5]nonan-2-yl)-5-methyl-1H-pyrazol-4-yl)-4-(2-hydroxy-1-(1-methyl-1H-pyrazol-3-yl)ethoxy)pyrazolo[1,5-a]pyridine-3-carbonitrile;
6-(1-(7-acryloyl-7-azaspiro[3.5]nonan-2-yl)-5-methyl-1H-pyrazol-4-yl)-4-(2-hydroxy-1-(isoquinolin-4-yl)ethoxy)pyrazolo[1,5-a]pyridine-3-carbonitrile;
4-(2-hydroxy-1-(pyridin-2-yl)ethoxy)-6-(5-methyl-1-(1-(1-propionylazetidine-3-carbonyl)piperidin-4-yl)-1H-1,2,3-triazol-4-yl)pyrazolo[1,5-a]pyridine-3-carbonitrile;
(R)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(S)-6-(1-(1-(1-acryloylazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(R)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(S)-6-(1-(1-(1-acryloyl-3-fluoroazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(R)-6-(1-(1-(1-acryloyl-3-methoxyazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(S)-6-(1-(1-(1-acryloyl-3-methoxyazetidine-3-carbonyl)piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
(E)-6-(1-(1-(1-(4-(dimethylamino)but-2-enoyl)-3-methoxyazetidine-3-carbonyl) piperidin-4-yl)-5-methyl-1H-1,2,3-triazol-4-yl)-4-(2-hydroxy-1-(pyridin-2-yl)ethoxy)pyrazolo[1,5-a]pyridine-3-carbonitrile;
4-(2-hydroxy-1-(pyridin-2-yl)ethoxy)-6-(1-(1-(3-methoxy-1-propioloylazetidine-3-carbonyl)piperidin-4-yl)-5-methyl-1H-1,2,3-triazol-4-yl)pyrazolo[1,5-a]pyridine-3-carbonitrile;
(R)-6-(1-(7-acryloyl-7-azaspiro[3.5]nonan-2-yl)-1H-pyrazol-4-yl)-4-(1-(5-fluoropyridin-2-yl)-2-hydroxyethoxy)pyrazolo[1,5-a]pyridine-3-carbonitrile;
(R)-4-(1-(5-fluoropyridin-2-yl)-2-hydroxyethoxy)-6-(1-(7-propionyl-7-azaspiro[3.5]nonan-2-yl)-1H-pyrazol-4-yl)pyrazolo[1,5-a]pyridine-3-carbonitrile;
(S)-6-(1-(7-acryloyl-7-azaspiro[3.5]nonan-2-yl)-1H-pyrazol-4-yl)-4-(1-(5-fluoropyridin-2-yl)-2-hydroxyethoxy)pyrazolo[1,5-a]pyridine-3-carbonitrile;
(S)-4-(1-(5-fluoropyridin-2-yl)-2-hydroxyethoxy)-6-(1-(7-propionyl-7-azaspiro[3.5]nonan-2-yl)-1H-pyrazol-4-yl)pyrazolo[1,5-a]pyridine-3-carbonitrile;
6-(1-(1-(1-acryloyl-3-fluoroazetidine-3-carbonyl)piperidin-4-yl)-5-methyl-1H-1,2,3-triazol-4-yl)-4-(2-(dimethylamino)-ethoxy)pyrazolo[1,5-a]pyridine-3-carbonitrile;
4-(2-(dimethylamino) ethoxy)-6-(1-(1-(3-fluoro-1-propionylazetidine-3-carbonyl) piperidin-4-yl)-5-methyl-1H-1,2,3-triazol-4-yl)pyrazolo[1,5-a]pyridine-3-carbonitrile;
or a pharmaceutically acceptable salt thereof.

56. A pharmaceutical composition comprising a compound, or a pharmaceutically acceptable salt thereof, according to claim 1, and a pharmaceutically acceptable carrier, diluent or excipient.

57. A method of treating systemic sclerosis, fibrosis, pulmonary fibrosis, achondroplasia, thanatophoric dysplasia, severe achondroplasia with developmental delay and acanthosis nigricans (SADDAN), muenke syndrome or cancer, comprising administering to a patient in need of such treatment an effective amount of a compound of claim 1, or a pharmaceutically acceptable salt thereof.

58. The method of claim 57 wherein the treatment is cancer and the cancer is selected from the group consisting of breast cancer, invasive ductal breast cancer, invasive lobular breast cancer, lung cancer, non-small-cell lung cancer, lung adenocarcinoma, squamous cell lung cancer, small-cell lung cancer, urothelial cancer, bladder cancer, urothelial bladder cancer, non-muscle invasive bladder cancer, muscle invasive bladder cancer, upper tract cancer, urothelial upper tract cancer, urethral cancer, gastric cancer, pancreatic cancer, prostate cancer, colorectal cancer, multiple myeloma, liver cancer, melanoma, cutaneous melanoma, head and neck cancer, oral cancer, thyroid cancer, renal cancer, renal pelvis cancer, glioblastoma, endometrial cancer, cervical cancer, ovarian cancer, and testicular cancer.

59-60. (canceled)

61. The method according to claim 57, wherein the cancer is FGFR3-associated cancer.

62-68. (canceled)
